(19) United States
(12) Patent Application Publication
(10) Pub. No.: US 2021/0322549 A1
(43) Pub. Date: Oct. 21, 2021

(54) Stable Aqueous Formulations of Natalizumab

(71) Applicant: Coherus Biosciences, Inc., Redwood City, CA (US)

(72) Inventors: Mark Manning, Johnstown, CO (US); Robert Walter Payne, Fort Collins, CO (US)

(21) Appl. No.: 15/745,365

(22) PCT Filed: Jul. 18, 2016
(86) PCT No.: PCT/US2016/042765

§ 371 Date: Jan. 16, 2018

Related U.S. Application Data
(60) Provisional application No. 62/193,977, filed on Jul. 17, 2015.

(51) Int. Cl. A61K 39/395; C07K 16/28; A61K 47/14; A61K 47/22; A61K 47/26

(52) U.S. Cl. CPC A61K 39/39591; C07K 16/2839; A61K 47/14; C07K 2317/94; A61K 47/26; C07K 2317/24; A61K 47/22

(57) ABSTRACT

Described herein is a stable aqueous pharmaceutical formulation comprising a therapeutically effective amount of natalizumab (optionally not subjected to prior lyophilization), a buffer effective to maintain the pH in the range from about 4.0 to about 7.0; and optionally, one or more surfactant{s), one or more amino acid(s), one or more sugar(s), one or more polyol(s), one or more chelating agent(s) (one or more polymer(s), one or more other stabilizing agent(s); and methods for making such a formulation; and methods of using such a formulation.

### TECHNICAL FIELD

The present invention relates to aqueous pharmaceutical compositions suitable for long-term storage of natalizumab (including antibody proteins considered or intended as “biosimilar” or “bio-better” variants of commercially available natalizumab), methods of manufacture of the compositions and methods of their administration.

### BACKGROUND

Inflammation is a response of vascularized tissues to infection or injury and is affected by adhesion of leukocytes to the endothelial cells of blood vessels and their infiltration into the surrounding tissues. In normal inflammation, infiltrating leukocytes release toxic mediators to kill invading organisms, phagocytize debris and dead cells, and play a role in tissue repair and the immune response. However, in pathologic inflammation, infiltrating leukocytes are over-responsive and can cause serious or fatal damage. See, e.g., Hickey, Psychoneuroimmunology II (Academic Press 1990).

The integrins are a family of cell-surface glycoproteins involved in cell-adhesion, immune cell migration and activation. Alpha-4 (a4) integrin is expressed by all circulating leukocytes except neutrophils, and forms heterodimeric receptors in conjunction with either the beta1 (β1) or beta7 (β7) integrin subunits. Both alpha-4 beta-1 (α4β1) and alpha-4 beta-7 (α4β7) play a role in the migration of leukocytes across the vascular endothelium (Springer et al., Cell, 1994 76: 301-14; Butcher et al., Science, 1996, 272: 60-6) and contribute to cell activation and survival within the parenchyma (Damle et al., J. Immunol., 1993; 151: 2368-79; Koopman et al., J. Immunol., 1994, 152: 3760-7; Leussink et al., Acta Neuropathol., 2002, 103: 131-136). α4β1 is constitutively expressed on lymphocytes, monocytes, macrophages, mast cells, basophils and eosinophils.

α4β1 (also known as very late antigen-4, VLA-4), binds to vascular cell adhesion molecule-1 (VCAM-1) (Lobb et al., J. Clin. Invest., 1994, 94: 1722-8), which is expressed by the vascular endothelium at many sites of chronic inflammation (Bevilacqua et al., 1993 Annu. Rev. Immunol., 11: 767-804; Postigo et al., 1993 Res. Immunol., 144: 723-35). α4β1 has other ligands, including fibronectin and other extracellular matrix (ECM) components.

Intercellular adhesion mediated by α4β1 and other cell surface receptors is associated with a number of inflammatory responses. At the site of an injury or other inflammatory stimulus, activated vascular endothelial cells express molecules that are adhesive for leukocytes. The mechanics of leukocyte adhesion to endothelial cells involves, in part, the recognition and binding of cell surface receptors on leukocytes to the corresponding cell surface molecules on endothelial cells. Once bound, the leukocytes migrate across the blood vessel wall to enter the injured site and release chemical mediators to combat infection.

Natalizumab is a recombinant, humanized form of a murine monoclonal antibody that binds to the α4 subunit of α4β1 (also known as very late antigen 4 [VLA-4] or CD49d-CD29) and α4β7 integrin. The α4-subunit of α4β1 and α4β7 integrins is expressed on the surface of all leukocytes except neutrophils, and inhibits the α4-mediated adhesion of leukocytes to their counter-receptor(s). The α4-integrins mediate several homing and adhesive functions. This is accomplished through the binding of the α4-integrins to their cognate receptors vascular cell adhesion molecule-1 (VCAM-1) and mucosal addressing cell adhesion molecule-1 (MadCAM-1) expressed on endothelium. MadCAM-1 binding is specific to the α4β7-integrin expressed primarily on T lymphocytes and monocytes in the intestine. Additional α4-integrin ligands include osteopontin and fibronectin (CS-1) expressed within the extracellular matrix.

In the past years, advances in biotechnology have made it possible to produce a variety of proteins for pharmaceutical applications using recombinant DNA techniques. Because proteins are larger and more complex than traditional organic and inorganic drugs (e.g., possessing multiple functional groups in addition to complex three-dimensional structures), the formulation of such proteins poses special problems. For a protein to remain biologically active, a formulation must preserve intact the conformational integrity of at least a core sequence of the protein's amino acids while at the same time protecting the protein's multiple functional groups from degradation. Degradation pathways for proteins can involve chemical instability (e.g., any process which involves modification of the protein by bond formation or cleavage resulting in a new chemical entity) or physical instability (e.g., changes in the higher order structure of the protein). Chemical instability can result from deamidation, racemization, hydrolysis, oxidation, beta elimination or disulfide exchange. Physical instability can result from denaturation, aggregation, precipitation or adsorption, for example. The three most common protein degradation pathways are protein aggregation, deamidation and oxidation. Cleland et al Critical Reviews in Therapeutic Drug Carrier Systems 10(4): 307-377 (1993).

Polypeptides must often be stored prior to their use. When stored for extended periods, polypeptides are frequently unstable in solution (Manning et al., 1989, Pharm. Res. 6:903-918). To extend their shelf life, additional processing steps have been developed, such as drying, e.g., lyophilization. However, lyophilized pharmaceutical compositions are less convenient to use.

Typical practices to improve polypeptide stability can be addressed by varying the concentration of elements with the formulation, or by adding excipients to modify the formulation (See, for example, U.S. Pat. Nos. 5,580,856 and 6,171,586). However, the use of additives can still result in inactive polypeptides. In addition, in the case of lyophilization, the rehydration step can result in inactivation of the polypeptide by, for example, aggregation or denaturation (Hora et al., 1992, Pharm. Res., 9:33-36; Liu et al., 1991, Biotechnol. Bioeng., 37:177-184). In fact, aggregation of polypeptides is undesirable, as it may result in immunogenicity (Cleland et al., 1993, Crit. Rev. Therapeutic Drug Carrier Systems, 10:307-377; and Robbins et al., 1987, Diabetes, 36:838-845).

Various formulations of natalizumab are known in the art. See, for example, U.S. Pat. No. 8,349,321 and US Patent Application US2013/0017193. However, there is still need for stable liquid formulations of natalizumab that allow its long term storage without substantial loss in efficacy.

### BRIEF SUMMARY OF THE INVENTION

The invention provides stable aqueous formulations comprising natalizumab, e.g., formulations that allow its long term storage.

In one aspect, the invention features an aqueous pharmaceutical composition, such as a stable aqueous pharmaceutical composition, containing (in a physiologically-acceptable buffer) an alpha-4 integrin binding antibody at a concentration of about 5 to about 200 mg/mL, e.g., 5 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, and 200 mg/mL; preferred concentrations about 20 mg/mL and about 150 mg/mL.

The pH of the formulations of the invention may be “self-buffered” by natalizumab itself, especially at higher natalizumab concentrations (e.g., at concentration equal to or greater than about 50 mg/mL, e.g., 150 mg/ml natalizumab), or may be buffered using an added buffer or buffer system (at any natalizumab concentration). In certain embodiments, the added buffer is selected from the group consisting of acetate (e.g., at a pH between about pH 4 and 5.5), citrate (e.g., at a pH between about pH 5 and 7), succinate (e.g., at a pH between about pH 5 and 7), histidine (e.g., at a pH between about pH 5 and 7.5), phosphate (e.g., at a pH between about pH 6 and 8), and Tris buffer (e.g., at a pH between about pH 7 and pH 8). Other buffers which one of skill in the art could employ include glycine, bicarbonate, tartrate and maleate, at corresponding pH ranges. Natalizumab was found to be particularly stable in a pH range between pH 4.5 and pH 6.5; particularly suitable added buffers include citrate, acetate, and histidine (see Examples, below).

The basic composition comprises natalizumab, optionally a buffer system at a specified pH, and optionally a stabilizer/tonicity modifier, keeping in mind that stabilizers contribute to the overall tonicity of the solution. In addition, the formulation may contain a surfactant, a chelating agent, a sacrificial additive, or some other excipient intended to improve stability, increase solubility, or decrease viscosity.

In one aspect, the invention provides a stable aqueous formulation comprising natalizumab and a stabilizer, wherein the stabilizer is selected from the group consisting of amino acids, sugars, polyols, polymers, and combinations thereof. In one embodiment, the stabilizer is at least one amino acid selected from glycine, alanine, arginine, serine, proline and glutamate. In a second embodiment, the stabilizer is selected from a sugar, polyol, a polymer and combinations thereof. In a third embodiment, the stabilizer is a polymer, such as dextran, a starch, a poly(ethylene glycol), or a combination thereof, alone or in conjunction with a sugar, polyols, or amino acid.

In another aspect, the invention provides a stable aqueous formulation comprising natalizumab, a buffer and a salt. In certain embodiments, the buffer is selected from the group consisting of acetate (e.g., at pH 4 to 5.5), citrate (e.g., at pH 5 to 6.5), histidine (e.g., at pH 5 to 7), phosphate (e.g., at pH 6 to 8), and Tris buffer (e.g., at pH 7 to 8). Other buffers which one of skill in the art could employ include succinate, histidine, tartrate and maleate, at corresponding pH ranges. In some embodiments, the salt is selected from NaCl, Na₂SO₄, and KCl. In certain embodiments, the formulation optionally comprises a polyol (e.g., mannitol) and NaCl. In some embodiments, the formulation comprises low levels of NaCl (e.g., less than about 60 mM) in the presence of glycine and mannitol.

In another aspect, the invention provides a stable aqueous formulation comprising natalizumab and a chelating agent and a sacrificial additive. The chelating agent is selected from EDTA, DPTA, etc. The sacrificial additive is selected from ascorbate, N-acetyl-tryptophan, and methionine (or a methionine derivative). In some embodiments, the pH is 5.0, 5.5, 6.0, 6.5, 7.0, 7.5, or 8.0. The formulation optionally comprises a buffer.

In a further aspect, the invention provides a stable aqueous formulation comprising natalizumab, a surfactant, optionally a buffer, and optionally a tonicity modifier. In some embodiments the surfactant is selected from polysorbate 20, polysorbate 80, SDS, DDM and poloxamer 188 (Pluronic F-68); experiments performed in support of the present invention demonstrate that PS 80, PS 20 and DDM confer particularly favorable properties to natalizumab formulations. In certain embodiments, the buffer is selected from the group consisting of acetate (e.g., at pH 4 to 5.5), citrate (e.g., at pH 5 to 6.5), histidine (e.g., at pH 5 to 7), phosphate (e.g., at pH 6 and 8), and Tris buffer (e.g., at pH 7 to 8). Other buffers which one of skill in the art could employ include succinate, histidine, tartrate, glycine, bicarbonate, and maleate, at corresponding pH ranges. In certain embodiments, the tonicity modifier is selected from a salt, a polyol, an amino acid, or combinations thereof.

In yet another embodiment, the invention provides a stable aqueous formulation comprising natalizumab, a sugar and a polyol. For example, the sugar may be sucrose and the polyol may be selected from the group consisting of mannitol and sorbitol.

In another embodiment, the sugar is trehalose and the polyol is selected from the group consisting of mannitol and sorbitol.

In another embodiment, the sugar is dextrose and the polyol is selected from the group consisting of mannitol and sorbitol.

In yet another embodiment, the invention provides a stable aqueous formulation comprising natalizumab and an excipient, wherein the excipient is selected from the group consisting of ionic polyol derivatives, such as meglumine, mannosylglycerate, glucosylglycerate, mannosyllactate, mannosylglycolate, and diglycerolphosphate.

These and other aspects will become apparent from the following description of the various embodiments, although variations and modifications therein may be affected without departing from the spirit and scope of the novel concepts of the disclosure.

### Exemplary Embodiments 1-194

1. An aqueous formulation having a pH of between 4 and 7, comprising between about 5 mg/mL and about 200 mg/mL natalizumab.
2. An aqueous formulation of embodiment 1, comprising about 5 mg/mL natalizumab.
3. An aqueous formulation of embodiment 1, comprising about 20 mg/mL natalizumab.
4. An aqueous formulation of embodiment 1, comprising about 150 mg/mL natalizumab.
5. An aqueous formulation of any of embodiments 1-4, further comprising a buffer.
6. An aqueous formulation of embodiment 5, wherein said buffer is not phosphate buffer.
7. An aqueous formulation of embodiment 5, wherein said buffer is selected from the group consisting of acetate, succinate, citrate, histidine, phosphate, tris, glycine, lactate, tartrate and maleate.
8. An aqueous formulation of embodiment 5, wherein said buffer is selected from the group consisting of acetate, succinate, citrate and histidine.
9. An aqueous formulation of embodiment 8, wherein said buffer is acetate at pH 4 to 6.
10. An aqueous formulation of embodiment 9, wherein said buffer is acetate at pH 4.5 to 5.5.
11. An aqueous formulation of embodiment 9, wherein said buffer is 5 mM to 50 mM acetate.
12. An aqueous formulation of embodiment 9, wherein said buffer is 10-30 mM acetate at about pH 4.5.
13. An aqueous formulation of embodiment 9, wherein said buffer is 10-30 mM acetate at about pH 5.
14. An aqueous formulation of embodiment 9, wherein said buffer is 10-30 mM acetate at about pH 5.1.
15. An aqueous formulation of embodiment 9, wherein said buffer is 10-30 mM acetate at about pH 5.2.
16. An aqueous formulation of embodiment 9, wherein said buffer is 10-30 mM acetate at about pH 5.3.
17. An aqueous formulation of embodiment 9, wherein said buffer is 10-30 mM acetate at about pH 5.4.
18. An aqueous formulation of embodiment 9, wherein said buffer is 10-30 mM acetate at about pH 5.5.
19. An aqueous formulation of embodiment 8, wherein said buffer is succinate at about pH 4.5 to 6.
20. An aqueous formulation of embodiment 19, wherein said buffer is 5 mM to 50 mM succinate.
21. An aqueous formulation of embodiment 19, wherein said buffer is 10-30 mM succinate at pH 5.
22. An aqueous formulation of embodiment 19, wherein said buffer is 10-30 mM succinate at pH 5.5.
23. An aqueous formulation of embodiment 8, wherein said buffer is citrate at pH 4.5 to 6.5.
24. An aqueous formulation of embodiment 23, wherein said buffer is citrate at pH 5 to 6.
25. An aqueous formulation of embodiment 23, wherein said buffer is 5 mM to 50 mM citrate.
26. An aqueous formulation of embodiment 23, wherein said buffer is 10-30 mM citrate at about pH 5.
27. An aqueous formulation of embodiment 23, wherein said buffer is 10-30 mM citrate at about pH 5.5.
28. An aqueous formulation of embodiment 23, wherein said buffer is 10-30 mM citrate at about pH 6.
29. An aqueous formulation of embodiment 8, wherein said buffer is histidine at pH 4.5 to 7.
30. An aqueous formulation of embodiment 29, wherein said buffer is histidine at pH 5 to 6.5.
31. An aqueous formulation of embodiment 29, wherein said buffer is 5 mM to 50 mM histidine.
32. An aqueous formulation of embodiment 29, wherein said buffer is 10-30 mM histidine at about pH 5.
33. An aqueous formulation of embodiment 29, wherein said buffer is 10-30 mM histidine at about pH 5.5.
34. An aqueous formulation of embodiment 29, wherein said buffer is 10-30 mM histidine at about pH 5.6.
35. An aqueous formulation of embodiment 29, wherein said buffer is 10-30 mM histidine at about pH 5.7.
36. An aqueous formulation of embodiment 29, wherein said buffer is 10-30 mM histidine at about pH 5.8.
37. An aqueous formulation of embodiment 29, wherein said buffer is 10-30 mM histidine at about pH 5.9.
38. An aqueous formulation of embodiment 29, wherein said buffer is 10-30 mM histidine at about pH 6.
39. An aqueous formulation of embodiment 29, wherein said buffer is 10-30 mM histidine at about pH 6.5.
40. An aqueous formulation of any of embodiments 1-39 or 170, further comprising a stabilizer or tonicity agent.
41. An aqueous formulation of embodiment 40, wherein said stabilizer or tonicity agent is selected from the group consisting of sugars, polyols, and amino acids.
42. An aqueous formulation of embodiment 41, wherein said stabilizer is a polyol selected from the group consisting of glycerol, xylitol, inositol, mannitol, and sorbitol.
43. An aqueous formulation of embodiment 42, wherein said polyol is mannitol or sorbitol at a concentration of 50 mM to 400 mM.
44. An aqueous formulation of embodiment 43, wherein said polyol is mannitol at a concentration of 150 mM to 300 mM.
45. An aqueous formulation of embodiment 43, wherein said polyol is sorbitol at a concentration of 150 mM to 300 mM.
46. An aqueous formulation of embodiment 41, wherein said sugar is selected from the group consisting of sucrose, trehalose, lactose, glucose, dextrose and maltose.
47. An aqueous formulation of embodiment 46, wherein said sugar is trehalose at a concentration of 20 mM to 400 mM.
48. An aqueous formulation of embodiment 47, wherein said sugar is trehalose at a concentration of 150 mM to 300 mM.
49. An aqueous formulation of embodiment 48, wherein said sugar is trehalose at a concentration of 250 mM to 300 mM.
50. An aqueous formulation of embodiment 46, wherein said sugar is sucrose at a concentration of 20 mM to 400 mM.
51. An aqueous formulation of embodiment 50, wherein said sugar is sucrose at a concentration of 150 mM to 300 mM.
52. An aqueous formulation of embodiment 51, wherein said sugar is sucrose at a concentration of 250 mM to 300 mM.
53. An aqueous formulation of any of embodiments 1-52, further comprising one or more amino acid(s).
54. An aqueous formulation of embodiment 53, wherein said amino acids are selected from the group consisting of glycine, arginine, glutamic acid, proline, threonine, lysine, aspartic acid and serine.
55. An aqueous formulation of embodiment 53, wherein said amino acids are selected from the group consisting of glycine, arginine and proline.
56. An aqueous formulation of embodiment 55, wherein one of said amino acids is glycine at a concentration of 50 mM to 300 mM.
57. An aqueous formulation of embodiment 56, wherein one of said amino acids is glycine at a concentration of 100 mM to 240 mM.
58. An aqueous formulation of embodiment 55, wherein one of said amino acids is arginine at a concentration of 10 mM to 100 mM.
59. An aqueous formulation of embodiment 58, wherein one of said amino acids is arginine at a concentration of 20 mM to 75 mM.
60. An aqueous formulation of embodiment 55, wherein one of said amino acids is proline at a concentration of 10 mM to 250 mM.
61. An aqueous formulation of embodiment 60, wherein one of said amino acids is proline at a concentration of 50 mM to 200 mM.
62. An aqueous formulation of any of embodiments 1-61, further comprising one or more salt(s).
63. An aqueous formulation of embodiment 62, wherein said salts are selected from the group consisting of NaCl, KCl, Na2SO4, MgCl2, and CaCl2.
64. An aqueous formulation of embodiment 63, wherein one of said salts is NaCl at a concentration of 10 mM to 200 mM.
65. An aqueous formulation of embodiment 64, wherein one of said salts is NaCl at a concentration of 50 mM to 150 mM.
66. An aqueous formulation of embodiment 65, wherein one of said salts is NaCl at a concentration of 130 mM to 150 mM.
67. An aqueous formulation of embodiment 66, wherein one of said salts is NaCl at a concentration of about 140 mM.
68. An aqueous formulation of any of embodiments 1-67, further comprising a surfactant.
69. An aqueous formulation of embodiment 68, wherein said surfactant is selected from the group consisting of Polysorbate 20 (PS20), Polysorbate 80 (PS80), poloxamer 188 (Pluronic F-68, or F-68), and DDM.
70. An aqueous formulation of embodiment 69, wherein said surfactant is polysorbate 20 at a concentration of 0.005% to 0.1%.
71. An aqueous formulation of embodiment 70, wherein said surfactant is polysorbate 20 at a concentration of about 0.01% to about 0.05%.
72. An aqueous formulation of embodiment 69, wherein said surfactant is polysorbate 80 at a concentration of 0.005% to 0.05%.
73. An aqueous formulation of embodiment 72, wherein said surfactant is polysorbate 80 at a concentration of about 0.01% to about 0.02%.
74. An aqueous formulation of embodiment 69, wherein said surfactant is DDM at a concentration of 0.05% to 0.5%.
75. An aqueous formulation of embodiment 74, wherein said surfactant is DDM at a concentration of about 0.1% to about 0.3%.
76. An aqueous formulation comprising natalizumab and a buffer, wherein said buffer is 5-40 mM (e.g., 20 mM) acetate at pH 4.
77. An aqueous formulation comprising natalizumab and a buffer, wherein said buffer is 5-40 mM (e.g., 20 mM) acetate at pH 4.5.
78. An aqueous formulation comprising natalizumab and a buffer, wherein said buffer is 5-40 mM (e.g., 20 mM) acetate at pH 5.
79. An aqueous formulation comprising natalizumab and a buffer, wherein said buffer is 5-40 mM (e.g., 20 mM) succinate at pH 5.
80. An aqueous formulation comprising natalizumab and a buffer, wherein said buffer is 5-40 mM (e.g., 20 mM) succinate at pH 5.5.
81. An aqueous formulation comprising natalizumab and a buffer, wherein said buffer is 5-40 mM (e.g., 20 mM) histidine at pH 5.
82. An aqueous formulation comprising natalizumab and a buffer, wherein said buffer is 5-40 mM (e.g., 20 mM) histidine at pH 5.5.
83. An aqueous formulation comprising natalizumab and a buffer, wherein said buffer is 5-40 mM (e.g., 20 mM) histidine at pH 6.5.
84. An aqueous formulation comprising natalizumab and a buffer, wherein said buffer is 5-40 mM (e.g., 20 mM) phosphate at pH 6.
85. An aqueous formulation comprising natalizumab and a buffer, wherein said buffer is 5-40 mM (e.g., 20 mM) phosphate at pH 6.5.
86. An aqueous formulation comprising natalizumab and a buffer, wherein said buffer is 5-40 mM (e.g., 20 mM) phosphate at pH 7.
87. An aqueous formulation comprising natalizumab and a buffer, wherein said buffer is 5-40 mM (e.g., 20 mM) tris at pH 7.
88. An aqueous formulation comprising natalizumab and a buffer, wherein said buffer is 5-40 mM (e.g., 20 mM) histidine at pH 5, and wherein the formulation further comprises 200-300 mM (e.g., 270 mM) trehalose and 0.01-0.04% (e.g., 0.02%) polysorbate 80.
89. An aqueous formulation comprising natalizumab and a buffer, wherein said buffer is 5-40 mM (e.g., 20 mM) histidine at pH 5.5, and wherein the formulation further comprises 200-300 mM (e.g., 270 mM) trehalose and 0.01-0.04% (e.g., 0.02%) polysorbate 80.
90. An aqueous formulation comprising natalizumab and a buffer, wherein said buffer is 5-40 mM (e.g., 20 mM) histidine at pH 5.9, and wherein the formulation further comprises 200-300 mM (e.g., 270 mM) trehalose and 0.01-0.04% (e.g., 0.02%) polysorbate 80.
91. An aqueous formulation comprising natalizumab and a buffer, wherein said buffer is 5-40 mM (e.g., 20 mM) histidine at pH 5, and wherein the formulation further comprises 200-300 mM (e.g., 270 mM) mannitol and 0.01-0.04% (e.g., 0.02%) polysorbate 80.
92. An aqueous formulation comprising natalizumab and a buffer, wherein said buffer is 5-40 mM (e.g., 20 mM) histidine at pH 5.5, and wherein the formulation further comprises 200-300 mM (e.g., 270 mM) mannitol and 0.01-0.04% (e.g., 0.02%) polysorbate 80.
93. An aqueous formulation comprising natalizumab and a buffer, wherein said buffer is 5-40 mM (e.g., 20 mM) histidine at pH 5.9, and wherein the formulation further comprises 200-300 mM (e.g., 270 mM) mannitol and 0.01-0.04% (e.g., 0.02%) polysorbate 80.
94. An aqueous formulation comprising natalizumab and a buffer, wherein said buffer is 5-40 mM (e.g., 20 mM) histidine at pH 5, and wherein the formulation further comprises 200-300 mM (e.g., 270 mM) sucrose and 0.01-0.04% (e.g., 0.02%) polysorbate 80.
95. An aqueous formulation comprising natalizumab and a buffer, wherein said buffer is 5-40 mM (e.g., 20 mM) histidine at pH 5.5, and wherein the formulation further comprises 200-300 mM (e.g., 270 mM) sucrose and 0.01-0.04% (e.g., 0.02%) polysorbate 80.
96. An aqueous formulation comprising natalizumab and a buffer, wherein said buffer is 5-40 mM (e.g., 20 mM) histidine at pH 5.9, and wherein the formulation further comprises 200-300 mM (e.g., 270 mM) sucrose and 0.01-0.04% (e.g., 0.02%) polysorbate 80.
97. An aqueous formulation comprising natalizumab and a buffer, wherein said buffer is 5-40 mM (e.g., 20 mM) histidine at pH 5, and wherein the formulation further comprises 200-300 mM (e.g., 270 mM) glycine and 0.01-0.04% (e.g., 0.02%) polysorbate 80.
98. An aqueous formulation comprising natalizumab and a buffer, wherein said buffer is 5-40 mM (e.g., 20 mM) histidine at pH 5.5, and wherein the formulation further comprises 200-300 mM (e.g., 270 mM) glycine and 0.01-0.04% (e.g., 0.02%) polysorbate 80.
99. An aqueous formulation comprising natalizumab and a buffer, wherein said buffer is 5-40 mM (e.g., 20 mM) histidine at pH 5.9, and wherein the formulation further comprises 200-300 mM (e.g., 270 mM) glycine and 0.01-0.04% (e.g., 0.02%) polysorbate 80.
100. An aqueous formulation comprising natalizumab and a buffer, wherein said buffer is 5-40 mM (e.g., 20 mM) histidine at pH 6, and wherein the formulation further comprises 25 mM arginine, 230 mM mannitol, and 0.01-0.04% (e.g., 0.02%) polysorbate 80.
101. An aqueous formulation comprising natalizumab and a buffer, wherein said buffer is 5-40 mM (e.g., 20 mM) histidine at pH 6, and wherein the formulation further comprises 50 mM arginine, 190 mM mannitol, and 0.01-0.04% (e.g., 0.02%) polysorbate 80.
102. An aqueous formulation comprising natalizumab and a buffer, wherein said buffer is 5-40 mM (e.g., 20 mM) histidine at pH 6.5, and wherein the formulation further comprises 200-300 mM (e.g., 270 mM) mannitol and 0.01-0.04% (e.g., 0.02%) polysorbate 80.
103. An aqueous formulation comprising natalizumab and a buffer, wherein said buffer is 5-40 mM (e.g., 20 mM) histidine at pH 6.5, and wherein the formulation further comprises 100-200 mM (e.g., 190 mM) sucrose and 0.01-0.04% (e.g., 0.02%)polysorbate 80.
104. An aqueous formulation comprising natalizumab and a buffer, wherein said buffer is 5-40 mM (e.g., 20 mM) histidine at pH 6.5, and wherein the formulation further comprises 100-200 mM (e.g., 140 mM) NaCl and 0.01-0.04% (e.g., 0.02%) polysorbate 80.
105. An aqueous formulation comprising natalizumab and a buffer, wherein said buffer is 5-40 mM (e.g., 20 mM) citrate at pH 5, and wherein the formulation further comprises 200-300 mM (e.g., 270 mM) mannitol and 0.01-0.04% (e.g., 0.02%) polysorbate 80.
106. An aqueous formulation comprising natalizumab and a buffer, wherein said buffer is 5-40 mM (e.g., 20 mM) citrate at pH 6, and wherein the formulation further comprises 200-300 mM (e.g., 270 mM) sorbitol and 0.01-0.04% (e.g., 0.02%) polysorbate 80.
107. An aqueous formulation comprising natalizumab and a buffer, wherein said buffer is 5-40 mM (e.g., 20 mM) acetate at pH 5, and wherein the formulation further comprises 200-300 mM (e.g., 270 mM) mannitol and 0.01-0.04% (e.g., 0.02%) polysorbate 80.
108. An aqueous formulation comprising natalizumab and a buffer, wherein said buffer is 5-40 mM (e.g., 20 mM) acetate at pH 5, and wherein the formulation further comprises 200-300 mM (e.g., 230 mM) glycine, 10-50 mM (e.g., 25 mM) arginine, and 0.01-0.04% (e.g., 0.02%) polysorbate 80.
109. An aqueous formulation comprising natalizumab and a buffer, wherein said buffer is 5-40 mM (e.g., 20 mM) acetate at pH 5.5, and wherein the formulation further comprises 200-300 mM (e.g., 270 mM) glycine and 0.01-0.04% (e.g., 0.02%) polysorbate 80.
110. An aqueous formulation comprising natalizumab and a buffer, wherein said buffer is 5-40 mM (e.g., 20 mM) acetate at pH 5.5, and wherein the formulation further comprises 200-300 mM (e.g., 270 mM) glycine, 10-100 mM (e.g., 50 mM) proline, 0.01-0.04% (e.g., 0.02%) polysorbate 80.
111. An aqueous formulation comprising natalizumab and a buffer, wherein said buffer is 5-40 mM (e.g., 20 mM) histidine at pH 5, and wherein the formulation further comprises 200-300 mM (e.g., 270 mM) trehalose and 0.05-0.4% (e.g., 0.1%) DDM.
112. An aqueous formulation comprising natalizumab and a buffer, wherein said buffer is 5-40 mM (e.g., 20 mM) histidine at pH 5.5, and wherein the formulation further comprises 200-300 mM (e.g., 270 mM) trehalose and 0.05-0.4% (e.g., 0.1%) DDM.
113. An aqueous formulation comprising natalizumab and a buffer, wherein said buffer is 5-40 mM (e.g., 20 mM) histidine at pH 5.9, and wherein the formulation further comprises 200-300 mM (e.g., 270 mM) trehalose and 0.05-0.4% (e.g., 0.1%) DDM.
114. An aqueous formulation comprising natalizumab and a buffer, wherein said buffer is 5-40 mM (e.g., 20 mM) histidine at pH 5, and wherein the formulation further comprises 200-300 mM (e.g., 270 mM) mannitol and 0.05-0.4% (e.g., 0.1%) DDM.
115. An aqueous formulation comprising natalizumab and a buffer, wherein said buffer is 5-40 mM (e.g., 20 mM) histidine at pH 5.5, and wherein the formulation further comprises 200-300 mM (e.g., 270 mM) mannitol and 0.05-0.4% (e.g., 0.1%) DDM.
116. An aqueous formulation comprising natalizumab and a buffer, wherein said buffer is 5-40 mM (e.g., 20 mM) histidine at pH 5.9, and wherein the formulation further comprises 200-300 mM (e.g., 270 mM) mannitol and 0.05-0.4% (e.g., 0.1%) DDM.
117. An aqueous formulation comprising natalizumab and a buffer, wherein said buffer is 5-40 mM (e.g., 20 mM) histidine at pH 5, and wherein the formulation further comprises 200-300 mM (e.g., 270 mM) sucrose and 0.05-0.4% (e.g., 0.1%) DDM.
118. An aqueous formulation comprising natalizumab and a buffer, wherein said buffer is 5-40 mM (e.g., 20 mM) histidine at pH 5.5, and wherein the formulation further comprises 200-300 mM (e.g., 270 mM) sucrose and 0.05-0.4% (e.g., 0.1%) DDM.
119. An aqueous formulation comprising natalizumab and a buffer, wherein said buffer is 5-40 mM (e.g., 20 mM) histidine at pH 5.9, and wherein the formulation further comprises 200-300 mM (e.g., 270 mM) sucrose and 0.05-0.4% (e.g., 0.1%) DDM.
120. An aqueous formulation comprising natalizumab and a buffer, wherein said buffer is 5-40 mM (e.g., 20 mM) histidine at pH 5, and wherein the formulation further comprises 200-300 mM (e.g., 270 mM) glycine and 0.05-0.4% (e.g., 0.1%) DDM.
121. An aqueous formulation comprising natalizumab and a buffer, wherein said buffer is 5-40 mM (e.g., 20 mM) histidine at pH 5.5, and wherein the formulation further comprises 200-300 mM (e.g., 270 mM) glycine and 0.05-0.4% (e.g., 0.1%) DDM.
122. An aqueous formulation comprising natalizumab and a buffer, wherein said buffer is 5-40 mM (e.g., 20 mM) histidine at pH 5.9, and wherein the formulation further comprises 200-300 mM (e.g., 270 mM) glycine and 0.05-0.4% (e.g., 0.1%) DDM.
123. An aqueous formulation comprising natalizumab and a buffer, wherein said buffer is 5-40 mM (e.g., 20 mM) histidine at pH 6, and wherein the formulation further comprises 25 mM arginine, 230 mM mannitol, and 0.05-0.4% (e.g., 0.1%) DDM.
124. An aqueous formulation comprising natalizumab and a buffer, wherein said buffer is 5-40 mM (e.g., 20 mM) histidine at pH 6, and wherein the formulation further comprises 50 mM arginine, 190 mM mannitol, and 0.05-0.4% (e.g., 0.1%) DDM.
125. An aqueous formulation comprising natalizumab and a buffer, wherein said buffer is 5-40 mM (e.g., 20 mM) histidine at pH 6.5, and wherein the formulation further comprises 200-300 mM (e.g., 270 mM) mannitol and 0.05-0.4% (e.g., 0.1%) DDM.
126. An aqueous formulation comprising natalizumab and a buffer, wherein said buffer is 5-40 mM (e.g., 20 mM) histidine at pH 6.5, and wherein the formulation further comprises 100-200 mM (e.g., 140 mM) NaCl and 0.05-0.4% (e.g., 0.1%) DDM.
127. An aqueous formulation comprising natalizumab and a buffer, wherein said buffer is 5-40 mM (e.g., 20 mM) citrate at pH 5, and wherein the formulation further comprises 200-300 mM (e.g., 270 mM) mannitol and 0.05-0.4% (e.g., 0.1%) DDM.
128. An aqueous formulation comprising natalizumab and a buffer, wherein said buffer is 5-40 mM (e.g., 20 mM) citrate at pH 6, and wherein the formulation further comprises 200-300 mM (e.g., 270 mM) sorbitol and 0.05-0.4% (e.g., 0.1%) DDM.
129. An aqueous formulation comprising natalizumab and a buffer, wherein said buffer is 5-40 mM (e.g., 20 mM) acetate at pH 5, and wherein the formulation further comprises 200-300 mM (e.g., 270 mM) mannitol and 0.05-0.4% (e.g., 0.1%) DDM.
130. An aqueous formulation comprising natalizumab and a buffer, wherein said buffer is 5-40 mM (e.g., 20 mM) acetate at pH 5, and wherein the formulation further comprises 200-300 mM (e.g., 230 mM) glycine, 10-50 mM (e.g., 25 mM) arginine, and 0.05-0.4% (e.g., 0.1%) DDM.
131. An aqueous formulation comprising natalizumab and a buffer, wherein said buffer is 5-40 mM (e.g., 20 mM) acetate at pH 5.5, and wherein the formulation further comprises 200-300 mM (e.g., 270 mM) glycine and 0.05-0.4% (e.g., 0.1%) DDM.
132. An aqueous formulation comprising natalizumab and a buffer, wherein said buffer is 5-40 mM (e.g., 20 mM) acetate at pH 5.5, and wherein the formulation further comprises 200-300 mM (e.g., 270 mM) glycine, 10-100 mM (e.g., 50 mM) proline and 0.05-0.4% (e.g., 0.1%) DDM.
133. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl.
134. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 0.005-0.04% (e.g., 0.01%) PS 80.
135. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 0.01-0.04% (e.g., 0.02%) PS 80.
136. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 0.005-0.05% (e.g., 0.01%) PS20.
137. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 0.01-0.05% (e.g., 0.02%) PS20.
138. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 0.05% PS20.
139. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 0.1% F68.
140. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 0.2% F68.
141. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 0.05 to 0.5% (e.g., 0.1%) DDM.
142. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl, and 0.25% DDM.
143. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 0.5% PEG 3350.
144. An aqueous formulation comprising natalizumab and a buffer, wherein said buffer is 5-40 mM (e.g., 20 mM) histidine at pH 6, and wherein the formulation further comprises 100-180 mM (e.g., 140 mM) NaCl and 1% PEG 3350.
145. An aqueous formulation comprising 20 mg/mL natalizumab and a buffer, wherein said buffer is 20 mM histidine at pH 5.5, and wherein the formulation further comprises 270 mM sucrose and 0.02% PS80.
146. An aqueous formulation comprising 20 mg/mL natalizumab and a buffer, wherein said buffer is 20 mM histidine at pH 5.5, and wherein the formulation further comprises 270 mM trehalose and 0.02% PS80.
147. An aqueous formulation comprising 20 mg/mL natalizumab and a buffer, wherein said buffer is 20 mM histidine at pH 5.5, and wherein the formulation further comprises 140 mM NaCl and 0.02% PS80.
148. An aqueous formulation comprising 5 mg/mL natalizumab and a buffer, wherein said buffer is 20 mM histidine at pH 5.5, and wherein the formulation further comprises 270 mM sucrose and 0.02% PS80.
149. An aqueous formulation comprising 20 mg/mL natalizumab and a buffer, wherein said buffer is 20 mM histidine at pH 5.9, and wherein the formulation further comprises 270 mM sucrose and 0.02% PS80.
150. An aqueous formulation comprising 20 mg/mL natalizumab and a buffer, wherein said buffer is 20 mM histidine at pH 5.9, and wherein the formulation further comprises 270 mM trehalose and 0.02% PS80.
151. An aqueous formulation comprising 20 mg/mL natalizumab and a buffer, wherein said buffer is 20 mM histidine at pH 5.9, and wherein the formulation further comprises 140 mM NaCl and 0.02% PS80.
152. An aqueous formulation comprising 20 mg/mL natalizumab and a buffer, wherein said buffer is 20 mM histidine at pH 5, and wherein the formulation further comprises 90 mM trehalose, 100 mM NaCl, and 0.02% PS80.
153. An aqueous formulation comprising 20 mg/mL natalizumab and a buffer, wherein said buffer is 20 mM histidine at pH 5.5, and wherein the formulation further comprises 90 mM trehalose, 100 mM NaCl, and 0.02% PS80.
154. An aqueous formulation comprising 20 mg/mL natalizumab and a buffer, wherein said buffer is 20 mM histidine at pH 5.9, and wherein the formulation further comprises 90 mM trehalose, 100 mM NaCl, and 0.02% PS80.
155. An aqueous formulation comprising 20 mg/mL natalizumab and a buffer, wherein said buffer is 20 mM histidine at pH 5.5, and wherein the formulation further comprises 180 mM proline, 50 mM NaCl, and 0.02% PS80.
156. An aqueous formulation comprising 20 mg/mL natalizumab and a buffer, wherein said buffer is 20 mM histidine at pH 5.9, and wherein the formulation further comprises 180 mM proline, 50 mM NaCl, and 0.02% PS80.
157. An aqueous formulation comprising 20 mg/mL natalizumab and a buffer, wherein said buffer is 20 mM histidine at pH 5.5, and wherein the formulation further comprises 230 mM sucrose, 25 mM NaCl, and 0.02% PS80.
158. An aqueous formulation comprising 20 mg/mL natalizumab and a buffer, wherein said buffer is 20 mM histidine at pH 5.9, and wherein the formulation further comprises 230 mM sucrose, 25 mM NaCl, and 0.02% PS80.
159. An aqueous formulation comprising 20 mg/mL natalizumab and a buffer, wherein said buffer is 20 mM acetate at pH 5.5, and wherein the formulation further comprises 110 mM sucrose, 110 mM proline, 25 mM NaCl, and 0.02% PS80.
160. An aqueous formulation comprising 5 mg/mL natalizumab and a buffer, wherein said buffer is 20 mM acetate at pH 5.5, and wherein the formulation further comprises 90 mM trehalose, 100 mM NaCl, and 0.02% PS80.
161. An aqueous formulation comprising 5 mg/mL natalizumab and a buffer, wherein said buffer is 20 mM acetate at pH 5.9, and wherein the formulation further comprises 80 mM proline, 100 mM NaCl, and 0.02% PS80.
162. An aqueous formulation comprising 20 mg/mL natalizumab and a buffer, wherein said buffer is 20 mM acetate at pH 5.5, and wherein the formulation further comprises 90 mM trehalose, 100 mM NaCl, and 0.02% PS80.
163. An aqueous formulation comprising 20 mg/mL natalizumab and a buffer, wherein said buffer is 20 mM acetate at pH 5.5, and wherein the formulation further comprises 80 mM proline, 100 mM NaCl, and 0.02% PS80.
164. An aqueous formulation comprising 20 mg/mL natalizumab and a buffer, wherein said buffer is 20 mM acetate at pH 5.9, and wherein the formulation further comprises 110 mM sucrose, 110 mM proline, 25 mM NaCl, and 0.02% PS80.
165. An aqueous formulation comprising 20 mg/mL natalizumab and a buffer, wherein said buffer is 20 mM acetate at pH 5.9, and wherein the formulation further comprises 90 mM trehalose, 100 mM NaCl, and 0.02% PS80.
166. An aqueous formulation comprising 20 mg/mL natalizumab and a buffer, wherein said buffer is 20 mM acetate at pH 5.9, and wherein the formulation further comprises 80 mM proline, 100 mM NaCl, and 0.02% PS80.
167. An aqueous formulation comprising 20 mg/mL natalizumab and a buffer, wherein said buffer is 20 mM acetate at pH 5, and wherein the formulation further comprises 240 mM trehalose, 25 mM NaCl, and 0.02% PS80.
168. An aqueous formulation comprising 20 mg/mL natalizumab and a buffer, wherein said buffer is 20 mM acetate at pH 5.5, and wherein the formulation further comprises 180 mM sucrose, 50 mM NaCl, and 0.02% PS80.
169. An aqueous formulation comprising 20 mg/mL natalizumab and a buffer, wherein said buffer is 20 mM acetate at pH 5.5, and wherein the formulation further comprises 240 mM trehalose, 25 mM NaCl, and 0.02% PS80.
170. An aqueous formulation comprising 20 mg/mL natalizumab and a buffer, wherein said buffer is 20 mM acetate at pH 5.9, and wherein the formulation further comprises 180 mM sucrose, 50 mM NaCl, and 0.02% PS80.
171. An aqueous formulation comprising 20 mg/mL natalizumab and a buffer, wherein said buffer is 20 mM acetate at pH 5.9 and wherein the formulation further comprises 240 mM trehalose, 25 mM NaCl, and 0.02% PS80.
172. An aqueous formulation comprising 5 mg/mL natalizumab and a buffer, wherein said buffer is 10 mM acetate at pH 5, and wherein the formulation further comprises 180 mM sucrose, 50 mM NaCl, and 0.02% PS80.
173. An aqueous formulation comprising 20 mg/mL natalizumab and a buffer, wherein said buffer is 10 mM acetate at pH 5, and wherein the formulation further comprises 240 mM trehalose, 25 mM NaCl, and 0.02% PS80.
174. An aqueous formulation comprising 20 mg/mL natalizumab and a buffer, wherein said buffer is 10 mM acetate at pH 5.5, and wherein the formulation further comprises 180 mM sucrose, 50 mM NaCl, and 0.02% PS80.
175. An aqueous formulation comprising 20 mg/mL natalizumab and a buffer, wherein said buffer is 10 mM acetate at pH 5.5, and wherein the formulation further comprises 240 mM trehalose, 25 mM NaCl, and 0.02% PS80.
176. An aqueous formulation comprising 20 mg/mL natalizumab and a buffer, wherein said buffer is 10 mM acetate at pH 5.9, and wherein the formulation further comprises 180 mM sucrose, 50 mM NaCl, and 0.02% PS80.
177. An aqueous formulation comprising 20 mg/mL natalizumab and a buffer, wherein said buffer is 10 mM acetate at pH 5.9 and wherein the formulation further comprises 240 mM trehalose, 25 mM NaCl, and 0.02% PS80.
178. An aqueous formulation according to any of embodiments 1-177, wherein said formulation is in a liquid state.
179. An aqueous formulation according to any of embodiments 1-177, wherein said formulation is in a frozen state.
180. An aqueous formulation according to any of embodiments 1-179, wherein said formulation is substantially stable.
181. An aqueous formulation according to embodiment 180, wherein said formulation is substantially stable for 1 week at 40° C., 2 weeks at 25° C., 4 weeks at 25° C., 8 weeks at 25° C., 16 weeks at 25° C., 6 months at 5° C., and/or 9 months 5° C.
182. An aqueous formulation according to embodiment 180, wherein said formulation is substantially stable for at least two weeks at 40° C.
183. An aqueous formulation according to embodiment 180, wherein said formulation is substantially stable for at least four weeks at 25° C.
184. An aqueous formulation according to embodiment 180, wherein said formulation is substantially stable for at least 13 weeks at 5° C.
185. An aqueous formulation according to embodiment 180, wherein said formulation is substantially stable following three freeze-thaw cycles.
186. An aqueous formulation according to embodiment 180, wherein said formulation is substantially stable following five freeze-thaw cycles.
187. An aqueous formulation according to embodiment 180, wherein said formulation is substantially stable following 24 hours of agitation on an orbital shaker.
188. An aqueous formulation according to embodiment 180, wherein said formulation is substantially stable following 48 hours of agitation on an orbital shaker.
189. An aqueous formulation according to any of embodiments 1-188, wherein said formulation is substantially free of SVPs.
190. An aqueous formulation according to embodiment 189, wherein said formulation has fewer than 12 SVPs per mL that are equal to or greater than 10 μm, and fewer than 2 per mL equal to or greater than 25 μm.
191. An aqueous formulation according to any of embodiments 1-190, wherein said formulation is substantially free of protein aggregates and/or high molecular weight species.
192. An aqueous formulation of any of embodiments 1-4, wherein said pH is buffered primarily by said natalizumab.
193. An aqueous formulation of embodiment 192, wherein said formulation is essentially buffer-free.
194. An aqueous formulation of any of embodiments 1-4, consisting essentially of natalizumab and water.

### Exemplary Embodiments A-GS

A. An aqueous formulation having a pH of between 4 and 9, comprising between about 10 mg/mL and about 200 mg/mL natalizumab.
B. An aqueous formulation of embodiment A, comprising about 20 mg/mL natalizumab.
C. An aqueous formulation of embodiment A, comprising about 150 mg/mL natalizumab.
D. An aqueous formulation of any of embodiments A-C, further comprising a buffer.
E. An aqueous formulation of embodiment 4, wherein said buffer is selected from the group consisting of sodium acetate, potassium acetate, succinate, sodium citrate, histidine, sodium phosphate, tris, and glycine.
F. An aqueous pharmaceutical composition of embodiment E, wherein said buffer is acetate at pH 4 to 6.
G. An aqueous pharmaceutical composition of embodiment F, wherein said buffer is 5 mM to 50 mM acetate.
H. An aqueous pharmaceutical composition of embodiment F, wherein said buffer is 10-30 mM acetate at pH 4.
I. An aqueous pharmaceutical composition of embodiment F, wherein said buffer is 10-30 mM acetate at pH 4.5.
J. An aqueous pharmaceutical composition of embodiment F, wherein said buffer is 10-30 mM acetate at pH 5.
K. An aqueous pharmaceutical composition of embodiment E, wherein said buffer is succinate at pH 4 to 6.
L. An aqueous pharmaceutical composition of embodiment K, wherein said buffer is 5 mM to 50 mM succinate.
M. An aqueous pharmaceutical composition of embodiment E, wherein said buffer is citrate at pH 4 to 6.5.
N. An aqueous pharmaceutical composition of embodiment M, wherein said buffer is 5 mM to 50 mM citrate.
O. An aqueous pharmaceutical composition of embodiment M, wherein said buffer is 10-30 mM citrate at pH 5.
P. An aqueous pharmaceutical composition of embodiment M, wherein said buffer is 10-30 mM citrate at pH 5.5.
Q. An aqueous pharmaceutical composition of embodiment E, wherein said buffer is histidine at pH 5 to 7.
R. An aqueous pharmaceutical composition of embodiment Q, wherein said buffer is 5 mM to 50 mM histidine.
S. An aqueous pharmaceutical composition of embodiment Q, wherein said buffer is 10-30 mM histidine at pH 5.
T. An aqueous pharmaceutical composition of embodiment Q, wherein said buffer is 10-30 mM histidine at pH 5.5.
U. An aqueous pharmaceutical composition of embodiment Q, wherein said buffer is 10-30 mM histidine at pH 6.5.
V. An aqueous pharmaceutical composition of embodiment E, wherein said buffer is phosphate at pH 6 to 8.
W. An aqueous pharmaceutical composition of embodiment V, wherein said buffer is 5 mM to 50 mM phosphate.
X. An aqueous pharmaceutical composition of embodiment V, wherein said buffer is 10-30 mM phosphate at pH 6.5.
Y. An aqueous pharmaceutical composition of embodiment V, wherein said buffer is 10-30 mM phosphate at pH 6.
Z. An aqueous pharmaceutical composition of embodiment V, wherein said buffer is 10-30 mM phosphate at pH 7.
AA. An aqueous pharmaceutical composition of embodiment V, wherein said buffer is 10-30 mM phosphate at pH 8.
AB. An aqueous pharmaceutical composition of embodiment V, wherein said buffer is 10-30 mM phosphate at pH 7.5.
AC. An aqueous pharmaceutical composition of embodiment E, wherein said buffer is tris at pH 7 to 9.
AD. An aqueous pharmaceutical composition of embodiment AC, wherein said buffer is 5 mM to 50 mM tris.
AE. An aqueous pharmaceutical composition of embodiment AC, wherein said buffer is 10-30 mM tris at pH 7.
AF. An aqueous pharmaceutical composition of embodiment AC, wherein said buffer is 10-30 mM tris at pH 7.5.
AG. An aqueous pharmaceutical composition of embodiment B, wherein said buffer is 10-30 mM tris at pH 8.
AH. An aqueous pharmaceutical composition of embodiment B, wherein said buffer is 10-30 mM tris at pH 8.5.
AI. An aqueous pharmaceutical composition of embodiment E, wherein said buffer is glycine at pH 7 to 9.
AJ. An aqueous pharmaceutical composition of embodiment AI, wherein said buffer is 5 mM to 50 mM glycine.
AK. An aqueous pharmaceutical composition of embodiment AI, wherein said buffer is 10-30 mM glycine at pH 9.
AL. An aqueous formulation of any of embodiments A to AK, further comprising a polyol and/or a sugar.
AM. An aqueous formulation of embodiment AL, wherein said polyol is selected from the group consisting of glycerol, xylitol, inositol, mannitol, and sorbitol.
AN. An aqueous formulation of embodiment AM, wherein said polyol is mannitol or sorbitol at a concentration of 50 mM to 400 mM.
AO. An aqueous formulation of embodiment AM, wherein said polyol is mannitol at a concentration of 150 mM or 220 mM.
AP. An aqueous formulation of embodiment AM, wherein said polyol is sorbitol at a concentration of 150 mM or 220 mM.
AQ. An aqueous formulation of embodiment AL, wherein said sugar is selected from the group consisting of sucrose, dextrose, trehalose, lactose, glucose, and maltose.
AR. An aqueous formulation of embodiment AQ wherein said sugar is trehalose at a concentration of 20 mM to 400 mM.
AS. An aqueous formulation of embodiment AQ, wherein said sugar is trehalose at a concentration of 65 mM or 220 mM.
AT. An aqueous formulation of any of embodiments A to AS, further comprising one or more amino acid(s).
AU. An aqueous formulation of embodiment AT, wherein said amino acids are selected from the group consisting of glycine, arginine, glutamic acid, proline and serine.
AV. An aqueous formulation of embodiment AU, wherein one of said amino acids is glycine at a concentration of 50 mM to 300 mM.
AW. An aqueous formulation of embodiment AV, wherein one of said amino acids is glycine at a concentration of 100 mM to 240 mM.
AX. An aqueous formulation of embodiment AU, wherein one of said amino acids is arginine at a concentration of 50 mM to 250 mM.
AY. An aqueous formulation of embodiment AX, wherein one of said amino acids is arginine at a concentration of 20 mM to 100 mM.
AZ. An aqueous formulation of embodiment AU, wherein one of said amino acids is proline at a concentration of 50 mM to 250 mM.
BA. An aqueous formulation of embodiment AZ, wherein one of said amino acids is proline at a concentration of 100 mM to 220 mM.
BB. An aqueous formulation of embodiment AU, wherein one of said amino acids is glutamic acid at a concentration of 10 mM to 100 mM.
BC. An aqueous formulation of embodiment BB, wherein one of said amino acids is glutamic acid at a concentration of 20 mM to 75 mM.
BD. An aqueous formulation of embodiment AU, wherein one of said amino acids is serine at a concentration of 50 mM to 250 mM.
BE. An aqueous formulation of embodiment BD, wherein one of said amino acids is serine at a concentration of 100 mM to 200 mM.
BF. An aqueous formulation of any of embodiments A to BE, further comprising one or more salt(s).
BG. An aqueous formulation of embodiment BF, wherein said salts are selected from the group consisting of NaCl, KCl, Na2SO4, MgCl2, and CaCl2.
BH. An aqueous formulation of embodiment BG, wherein one of said salts is NaCl at a concentration of 10 mM to 200 mM.
BI. An aqueous formulation of embodiment BH, wherein one of said salts is NaCl at a concentration of 50 mM to 100 mM.
BJ. An aqueous formulation of embodiment BG, wherein one of said salts is KCl at a concentration of 1 mM to 25 mM.
BK. An aqueous formulation of embodiment AZ, wherein one of said salts is KCl at a concentration of 10 mM.
BL. An aqueous formulation of embodiment BG, wherein one of said salts is Na2SO4 at a concentration of 50 mM to 200 mM.
BM. An aqueous formulation of embodiment BL, wherein one of said salts is Na2SO4 at a concentration of 80 mM to 100 mM.
BN. An aqueous formulation of embodiment BG, wherein one of said salts is MgCl2 at a concentration of 1 mM to 25 mM.
BO. An aqueous formulation of embodiment BN, wherein one of said salts is MgCl2 at a concentration of 5 mM to 10 mM.
BP. An aqueous formulation of embodiment BG, wherein one of said salts is CaCl2 at a concentration of 1 mM to 25 mM.
BQ. An aqueous formulation of embodiment BP, wherein one of said salts is CaCl2 at a concentration of 5 mM to 10 mM.
BR. An aqueous formulation of any of embodiments A to BQ, further comprising a polymer.
BS. An aqueous formulation of embodiment BR, wherein said polymer is selected from the group consisting of dextran, betaine, PVP, glycerol, and HES.
BT. An aqueous formulation of embodiment BS, wherein said polymer is 40 kD dextran at a concentration of 2% to 20%.
BU. An aqueous formulation of embodiment BT, wherein said polymer is 40 kD dextran at a concentration of 10%.
BV. An aqueous formulation of embodiment BS, wherein said polymer is betaine at a concentration of 20 mM to 200 mM.
BW. An aqueous formulation of embodiment BV, wherein said polymer is betaine at a concentration of 100 mM.
BX. An aqueous formulation of embodiment BS, wherein said polymer is PVP at a concentration of 2% to 20%.
BY. An aqueous formulation of embodiment BX, wherein said polymer is PVP at a concentration of 5% to 10%.
BZ. An aqueous formulation of embodiment BS, wherein said polymer is glycerol at a concentration of 20 mM to 200 mM.
CA. An aqueous formulation of embodiment BZ, wherein said polymer is glycerol at a concentration of 50 mM to 100 mM.
CB. An aqueous formulation of embodiment BS, wherein said polymer is HES at a concentration of 2% to 20%
CC. An aqueous formulation of embodiment CB, wherein said polymer is HES at a concentration of 5% to 10%
CD. An aqueous formulation of any of embodiments A to CC, further comprising a chelating agent.
CE. An aqueous formulation of embodiment CD, wherein said chelating agent is selected from the group consisting of EDTA and DPTA.
CF. An aqueous formulation of embodiment CE, wherein said chelating agent is EDTA at a concentration of 0.01 mM to 2 mM.
CG. An aqueous formulation of embodiment CF, wherein said chelating agent is EDTA at a concentration of 0.1 mM to 0.5 mM.
CH. An aqueous formulation of embodiment CE, wherein said chelating agent is DPTA at a concentration of 0.01 mM to 2 mM.
CI. An aqueous formulation of embodiment CH, wherein said chelating agent is DPTA at a concentration of 0.1 mM to 0.5 mM.
CJ. An aqueous formulation of any of embodiments A to CI, further comprising a sacrificial additive.
CK. An aqueous formulation of embodiment 0, wherein said sacrificial additive is selected from the group consisting of Met, N-Ac-Trp, and ascorbate.
CL. An aqueous formulation of embodiment CK, wherein said sacrificial additive is Met at a concentration of 2 mM to 20 mM.
CM. An aqueous formulation of embodiment CL, wherein said sacrificial additive is Met at a concentration of 5 mM.
CN. An aqueous formulation of embodiment CK, wherein said sacrificial additive is N-Ac-Trp at a concentration of 1 mM to 10 mM.
CO. An aqueous formulation of embodiment CN, wherein said sacrificial additive is N-Ac-Trp at a concentration of 5 mM.
CP. An aqueous formulation of embodiment CK, wherein said sacrificial additive is ascorbate at a concentration of 5 mM to 50 mM.
CQ. An aqueous formulation of embodiment CP, wherein said sacrificial additive is ascorbate at a concentration of 10 mM to 20 mM.
CR. An aqueous formulation of any of embodiments A to CQ, further comprising a surfactant.
CS. An aqueous formulation of embodiment CR, wherein said surfactant is selected from the group consisting of polysorbate 20, polysorbate 80, SDS, and poloxamer 188 (Pluronic F-68).
CT. An aqueous formulation of embodiment CS, wherein said surfactant is polysorbate 20 at a concentration of 0.005% to 0.04%.
CU. An aqueous formulation of embodiment CT, wherein said surfactant is polysorbate 20 at a concentration of 0.02% to 0.1%.
CV. An aqueous formulation of embodiment CS, wherein said surfactant is polysorbate 80 at a concentration of 0.005% to 0.04%.
CW. An aqueous formulation of embodiment CV, wherein said surfactant is polysorbate 80 at a concentration of 0.02% to 0.1%.
CX. An aqueous formulation of embodiment CS, wherein said surfactant is poloxamer 188 (Pluronic F-68) at a concentration of 0.05% to 0.5%.
CY. An aqueous formulation of embodiment CX, wherein said surfactant is poloxamer 188 (Pluronic F-68) at a concentration of 0.1%.
CZ. An aqueous pharmaceutical composition comprising natalizumab and a buffer, wherein said buffer is 20 mM histidine at pH 5, and wherein the composition further comprises 220 mM mannitol, and 0.02% polysorbate 80.
DA. An aqueous pharmaceutical composition comprising natalizumab and a buffer, wherein said buffer is 20 mM histidine at pH 5, and wherein the composition further comprises 220 mM mannitol.
DB. An aqueous pharmaceutical composition comprising natalizumab and a buffer, wherein said buffer is 20 mM histidine at pH 6, and wherein the composition further comprises 220 mM mannitol, and 0.02% polysorbate 80.
DC. An aqueous pharmaceutical composition comprising natalizumab and a buffer, wherein said buffer is 20 mM histidine at pH 6, and wherein the composition further comprises 220 mM mannitol.
DD. An aqueous pharmaceutical composition comprising natalizumab and a buffer, wherein said buffer is 20 mM histidine at pH 5, and wherein the composition further comprises 150 mM mannitol, 50 mM NaCl, and 0.02% polysorbate 80.
DE. An aqueous pharmaceutical composition comprising natalizumab and a buffer, wherein said buffer is 20 mM histidine at pH 5, and wherein the composition further comprises 150 mM mannitol, and 50 mM NaCl.
DF. An aqueous pharmaceutical composition comprising natalizumab and a buffer, wherein said buffer is 20 mM histidine at pH 5, and wherein the composition further comprises 220 mM sorbitol, and 0.02% polysorbate 80.
DG. An aqueous pharmaceutical composition comprising natalizumab and a buffer, wherein said buffer is 20 mM histidine at pH 5, and wherein the composition further comprises 220 mM sorbitol.
DH. An aqueous pharmaceutical composition comprising natalizumab and a buffer, wherein said buffer is 20 mM histidine at pH 5, and wherein the composition further comprises 220 mM trehalose, and 0.02% polysorbate 80.
DI. An aqueous pharmaceutical composition comprising natalizumab and a buffer, wherein said buffer is 20 mM histidine at pH 5, and wherein the composition further comprises 220 mM trehalose.
DJ. An aqueous pharmaceutical composition comprising natalizumab and a buffer, wherein said buffer is 20 mM histidine at pH 6, and wherein the composition further comprises 65 mM sorbitol, 100 mM NaCl, and 0.02% polysorbate 80.
DK. An aqueous pharmaceutical composition comprising natalizumab and a buffer, wherein said buffer is 20 mM histidine at pH 6, and wherein the composition further comprises 65 mM trehalose, 100 mM NaCl, and 0.02% polysorbate 80.
DL. An aqueous pharmaceutical composition comprising natalizumab and a buffer, wherein said buffer is 20 mM citrate at pH 5, and wherein the composition further comprises 220 mM mannitol, and 0.02% polysorbate 80.
DM. An aqueous pharmaceutical composition comprising natalizumab and a buffer, wherein said buffer is 20 mM citrate at pH 5, and wherein the composition further comprises 220 mM mannitol.
DN. An aqueous pharmaceutical composition comprising natalizumab and a buffer, wherein said buffer is 20 mM citrate at pH 6, and wherein the composition further comprises 220 mM sorbitol, and 0.02% polysorbate 80.
DO. An aqueous pharmaceutical composition comprising natalizumab and a buffer, wherein said buffer is 20 mM citrate at pH 6, and wherein the composition further comprises 220 mM trehalose, and 0.02% polysorbate 80.
DP. An aqueous pharmaceutical composition comprising natalizumab and a buffer, wherein said buffer is 20 mM citrate at pH 7, and wherein the composition further comprises 220 mM mannitol, and 0.02% polysorbate 80.
DQ. An aqueous pharmaceutical composition comprising natalizumab and a buffer, wherein said buffer is 10 mM acetate at pH 5.5, and wherein the composition further comprises 200 mM glycine, 20 mM arginine, and 0.02% polysorbate 80.
DR. An aqueous pharmaceutical composition comprising natalizumab and a buffer, wherein said buffer is 10 mM acetate at pH 5.5, and wherein the composition further comprises 20 mM arginine, 200 mM proline, and 0.02% polysorbate 80.
DS. An aqueous pharmaceutical composition comprising natalizumab and a buffer, wherein said buffer is 20 mM succinate at pH 5.5, and wherein the composition further comprises 220 mM glycine, and 0.02% polysorbate 80.
DT. An aqueous pharmaceutical composition comprising natalizumab and a buffer, wherein said buffer is 20 mM succinate at pH 5.5, and wherein the composition further comprises 220 mM arginine, and 0.02% polysorbate 80.
DU. An aqueous pharmaceutical composition comprising natalizumab and a buffer, wherein said buffer is 20 mM succinate at pH 5.5, and wherein the composition further comprises 220 mM proline, and 0.02% polysorbate 80.
DV. An aqueous pharmaceutical composition comprising natalizumab and a buffer, wherein said buffer is 20 mM succinate at pH 5.5, and wherein the composition further comprises 220 mM glutamic acid, and 0.02% polysorbate 80.
DW. An aqueous pharmaceutical composition comprising natalizumab and a buffer, wherein said buffer is 20 mM succinate at pH 4.5, and wherein the composition further comprises 220 mM glutamic acid, and 0.02% polysorbate 80.
DX. An aqueous pharmaceutical composition comprising natalizumab and a buffer, wherein said buffer is 20 mM succinate at pH 4.5, and wherein the composition further comprises 200 mM glycine, 20 mM arginine, and 0.02% polysorbate 80.
DY. An aqueous pharmaceutical composition comprising natalizumab and a buffer, wherein said buffer is 20 mM succinate at pH 4.5, and wherein the composition further comprises 200 mM glycine, 20 mM glutamic acid, and 0.02% polysorbate 80.
DZ. An aqueous pharmaceutical composition comprising natalizumab and a buffer, wherein said buffer is 20 mM succinate at pH 6.5, and wherein the composition further comprises 200 mM glycine, 20 mM glutamic acid, and 0.02% polysorbate 80.
EA. An aqueous pharmaceutical composition comprising natalizumab and a buffer, wherein said buffer is 20 mM succinate at pH 6.5, and wherein the composition further comprises 100 mM glycine, 75 mM glutamic acid, and 0.02% polysorbate 80.
EB. An aqueous pharmaceutical composition comprising natalizumab and a buffer, wherein said buffer is 20 mM succinate at pH 5.5, and wherein the composition further comprises 100 mM glycine, 75 mM glutamic acid, and 0.02% polysorbate 80.
EC. An aqueous pharmaceutical composition comprising natalizumab and a buffer, wherein said buffer is 20 mM histidine at pH 5.5, and wherein the composition further comprises 50 mM arginine, 50 mM glutamic acid, and 0.02% polysorbate 80.
ED. An aqueous pharmaceutical composition comprising natalizumab and a buffer, wherein said buffer is 20 mM histidine at pH 6.5, and wherein the composition further comprises 240 mM glycine, and 0.02% polysorbate 80.
EE. An aqueous pharmaceutical composition comprising natalizumab and a buffer, wherein said buffer is 20 mM histidine at pH 7.5, and wherein the composition further comprises 240 mM glycine, and 0.02% polysorbate 80.
EF. An aqueous pharmaceutical composition comprising natalizumab and a buffer, wherein said buffer is 20 mM histidine at pH 6.5, and wherein the composition further comprises 220 mM proline, and 0.02% polysorbate 80.
EG. An aqueous pharmaceutical composition comprising natalizumab and a buffer, wherein said buffer is 20 mM histidine at pH 5.5, and wherein the composition further comprises 240 mM glycine, and 0.02% polysorbate 80.
EH. An aqueous pharmaceutical composition comprising natalizumab and a buffer, wherein said buffer is 20 mM histidine at pH 5.5, and wherein the composition further comprises 200 mM glycine, 20 mM glutamic acid, and 0.02% polysorbate 80.
EI. An aqueous pharmaceutical composition comprising natalizumab and a buffer, wherein said buffer is 20 mM histidine at pH 5.5, and wherein the composition further comprises 100 mM glycine, 100 mM arginine, and 0.02% polysorbate 80.
EJ. An aqueous pharmaceutical composition comprising natalizumab and a buffer, wherein said buffer is 20 mM histidine at pH 5.5, and wherein the composition further comprises 100 mM glycine, 100 mM arginine, and 0.02% polysorbate 80.
EK. An aqueous pharmaceutical composition comprising natalizumab and a buffer, wherein said buffer is 20 mM histidine at pH 5.5, and wherein the composition further comprises 150 mM glycine, 50 mM arginine, and 0.02% polysorbate 80.
EL. An aqueous pharmaceutical composition comprising natalizumab and a buffer, wherein said buffer is 20 mM His at pH 5.5, further comprising 0.02% polysorbate 80.
EM. An aqueous pharmaceutical composition according to any of embodiments A-EL, further comprising 140 mM NaCl.
EN. An aqueous pharmaceutical composition according to any of embodiments A-EL, further comprising 130 mM NaCl, and 10 mM MgCl2.
EQ. An aqueous pharmaceutical composition according to any of embodiments A-EL, further comprising 130 mM NaCl, and 10 mM CaCl2.
EP. An aqueous pharmaceutical composition according to any of embodiments A-EL, further comprising 100 mM Na2SO4.
EQ. An aqueous pharmaceutical composition according to any of embodiments A-EL, further comprising 130 mM NaCl, and 10 mM KCl.
ER. An aqueous pharmaceutical composition according to any of embodiments A-EL, further comprising 120 mM Na2SO4, and 10 mM KCl.
ES. An aqueous pharmaceutical composition according to any of embodiments A-EL, further comprising 100 mM Na2SO4, and 10 mM MgCl2.
ET. An aqueous pharmaceutical composition according to any of embodiments A-EL, further comprising 20 mM NaCl, and 80 mM Na2SO4.
EU. An aqueous pharmaceutical composition according to any of embodiments A-EL, further comprising 100 mM Na2SO4, and 10 mM CaCl2.
EV. An aqueous pharmaceutical composition according to any of embodiments A-EL, further comprising 140 mM NaCl, and 5 mM MgCl2.
EW. An aqueous pharmaceutical composition according to any of embodiments A-EL, further comprising 140 mM NaCl, and 5 mM CaCl2.
EX. An aqueous pharmaceutical composition according to any of embodiments A-EW, further comprising 5% dextran, 200 mM mannitol.
EY. An aqueous pharmaceutical composition according to any of embodiments A-EW, further comprising 100 mM betaine.
EZ. An aqueous pharmaceutical composition according to any of embodiments A-EW, further comprising 100 mM serine.
FA. An aqueous pharmaceutical composition according to any of embodiments A-EW, further comprising 200 mM serine.
FB. An aqueous pharmaceutical composition according to any of embodiments A-EW, further comprising 5% PVP, and 200 mM mannitol.
FC. An aqueous pharmaceutical composition according to any of embodiments A-EW, further comprising 100 mM serine, and 100 mM mannitol.
FD. An aqueous pharmaceutical composition according to any of embodiments A-EW, further comprising 50 mM glycerol, and 150 mM mannitol.
FE. An aqueous pharmaceutical composition according to any of embodiments A-EW, further comprising 100 mM glycerol, and 100 mM mannitol.
FF. An aqueous pharmaceutical composition according to any of embodiments A-EW, further comprising 10% dextran, and 150 mM mannitol.
FG. An aqueous pharmaceutical composition according to any of embodiments A-EW, further comprising 10% PVP, and 150 mM mannitol.
FH. An aqueous pharmaceutical composition according to any of embodiments A-EW, further comprising 200 mM mannitol, and 5% HES.
FI. An aqueous pharmaceutical composition according to any of embodiments A-EW, further comprising 150 mM mannitol, and 10% HES.
FJ. An aqueous pharmaceutical composition according to any of embodiments A-FI, further comprising 0.1 mM EDTA, and 200 mM mannitol.
FK. An aqueous pharmaceutical composition according to any of embodiments A-FI, further comprising 0.5 mM EDTA, and 200 mM mannitol.
FL. An aqueous pharmaceutical composition according to any of embodiments A-FI, further comprising 0.1 mM DPTA, and 200 mM mannitol.
FM. An aqueous pharmaceutical composition according to any of embodiments A-FI, further comprising 0.5 mM DPTA, and 200 mM mannitol.
FN. An aqueous pharmaceutical composition according to any of embodiments A-FI, further comprising 5 mg/ml Met, and 200 mM mannitol.
FO. An aqueous pharmaceutical composition according to any of embodiments A-FI, further comprising 5 mg/ml N-Ac-Trp, and 200 mM mannitol.
FP. An aqueous pharmaceutical composition according to any of embodiments A-FI, further comprising 10 mM ascorbate, and 200 mM mannitol.
FQ. An aqueous pharmaceutical composition according to any of embodiments A-FI, further comprising 20 mM ascorbate, and 200 mM mannitol.
FR. An aqueous pharmaceutical composition according to any of embodiments A-FI, further comprising 0.1 mM EDTA, 5 mg/ml Met, and 200 mM mannitol.
FS. An aqueous pharmaceutical composition according to any of embodiments A-FI, further comprising 10 mg/ml Met, and 200 mM mannitol.
FT. An aqueous pharmaceutical composition according to any of embodiments A-FI, further comprising 5 mg/ml N-Ac-Trp, 10 mM ascorbate, and 180 mM mannitol.
FU. An aqueous pharmaceutical composition according to any of embodiments A-FI, further comprising 5 mg/ml Met, 10 mM ascorbate, and 180 mM mannitol.
FV. An aqueous pharmaceutical composition comprising natalizumab and a buffer, wherein said buffer is 20 mM histidine at pH 5.5, and wherein the composition further comprises 65 mM mannitol, 100 mM NaCl, and 0.02% polysorbate 80.
FW. An aqueous pharmaceutical composition comprising natalizumab and a buffer, wherein said buffer is 20 mM histidine at pH 5.5, and wherein the composition further comprises 65 mM mannitol, 100 mM NaCl, % polysorbate 20, and 0.1% polysorbate 80.
FX. An aqueous pharmaceutical composition comprising natalizumab and a buffer, wherein said buffer is 20 mM histidine at pH 5.5, and wherein the composition further comprises 65 mM mannitol, 100 mM NaCl, and 0.02% polysorbate 20.
FY. An aqueous pharmaceutical composition comprising natalizumab and a buffer, wherein said buffer is 20 mM histidine at pH 5.5, and wherein the composition further comprises 65 mM mannitol, 100 mM NaCl, and 0.01% polysorbate 20.
FZ. An aqueous pharmaceutical composition comprising natalizumab and a buffer, wherein said buffer is 20 mM histidine at pH 5.5, and wherein the composition further comprises 65 mM mannitol, 100 mM NaCl, and 0.1% F68.
GA. An aqueous pharmaceutical composition comprising natalizumab and a buffer, wherein said buffer is 20 mM histidine at pH 5.5, and wherein the composition further comprises 65 mM mannitol, and 100 mM NaCl.
GB. An aqueous pharmaceutical composition comprising natalizumab and a buffer, wherein said buffer is 20 mM histidine at pH 5.5, and wherein the composition further comprises 200 mM mannitol, and 0.02% polysorbate 80.
GC. An aqueous pharmaceutical composition comprising natalizumab and a buffer, wherein said buffer is 20 mM histidine at pH 5.5, and wherein the composition further comprises 200 mM mannitol, and 0.1% F68.
GD. An aqueous pharmaceutical composition comprising natalizumab and a buffer, wherein said buffer is 20 mM histidine at pH 5.5, and wherein the composition further comprises 200 mM mannitol, and 0.05% polysorbate 20.
GE. An aqueous pharmaceutical composition comprising natalizumab and a buffer, wherein said buffer is 20 mM histidine at pH 5.5, and wherein the composition further comprises 200 mM mannitol, and 0.1% polysorbate 80.
GF. An aqueous pharmaceutical composition comprising natalizumab and a buffer, wherein said buffer is 20 mM succinate at pH 5.5, and wherein the composition further comprises 65 mM mannitol, 100 mM NaCl, and 0.02% polysorbate 80.
GG. An aqueous pharmaceutical composition comprising natalizumab and a buffer, wherein said buffer is 20 mM succinate at pH 4.5, and wherein the composition further comprises 6.5 mM mannitol, 100 mM NaCl, and 0.02% polysorbate 80.
GH. An aqueous pharmaceutical composition according to any of embodiments A-GG, wherein said composition is substantially free of SVPs.
GI. An aqueous pharmaceutical composition according to any of embodiments A-GH, wherein said composition is substantially free of protein aggregates and/or high molecular weight species.
GJ. An aqueous pharmaceutical composition according to any of embodiments A to GI wherein the stability is characterized by a lack of particulates in the composition comparable to, or better than that of commercially available natalizumab.
GK. A stable aqueous formulation of natalizumab having a pH of between 4 and 8 that does not contain polyol or surfactant.
GL. A stable aqueous formulation of natalizumab having a pH of between 4 and 8 that does not contain a buffer.
GM. A stable aqueous formulation of natalizumab having a pH of between 4 and 8 that does not contain a salt.
GN. A stable aqueous formulation of natalizumab having a pH of between 4 and 8 that does not contain buffer, polyol or surfactant.
GO. An aqueous pharmaceutical composition according to any of embodiments A-GN, wherein said composition is stable.
GP. An aqueous pharmaceutical composition according to embodiment GO, wherein said composition is stable for 1 week at 40° C., 2 weeks at 40° C., 3 weeks at 40° C., 4 weeks at 40° C., 2 weeks at 25° C., 4 weeks at 25° C., 8 weeks at 25° C., 16 weeks at 25° C., 6 months at 25° C., 9 months at 25° C., 6 months at 2-8° C., e.g., 4° C., 9 months 2-8° C., e.g., 4° C., 12 months 2-8° C., e.g., 4° C., 18 months 2-8° C., e.g., 4° C., and/or 24 months 2-8° C., e.g., 4° C.
GQ. An aqueous pharmaceutical composition of embodiment GO or GP, wherein said composition does not lose more than 20%, more than 15%, more than 10%, or more than 5% of its activity relative to activity of the composition at the beginning of storage.
GR. An aqueous formulation of any of embodiments A to GQ, wherein said formulation is stable during long-term storage and/or exhibits long-term stability.
GS. An aqueous formulation of any of embodiments A to GR, wherein said formulation is stable after having been frozen.

It is to be understood that both the foregoing general description and the following detailed description are exemplary and explanatory only and are not restrictive of the invention, as claimed.

Other objects, features and advantages of the present invention will become apparent from the following detailed description. It should be understood, however, that the detailed description and specific examples, while indicating suitable embodiments of the invention, are given by way of illustration only, since various changes and modifications within the scope and spirit of the invention will become apparent to one skilled in the art from this detailed description.

### DETAILED DESCRIPTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. In the case of conflict, the present document, including definitions will control.

Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. It is to be understood that this invention is not limited to the particular methodology, protocols, and reagents described, as these may vary.

The headings provided herein are not limitations of the various aspects or embodiments of the invention which can be had by reference to the specification as a whole. Accordingly, the terms defined immediately below are more fully defined by reference to the specification as a whole.

### Definitions

The term “natalizumab” is intended to be synonymous with the active pharmaceutical ingredient in Tysabri® as well as protein considered or intended as a biosimilar or bio-better variant thereof. Natalizumab is a humanized IgG4/κ monoclonal anti-alpha-4 integrin antibody. It refers to a polypeptide which is an “alpha-4 integrin binding antibody.” An alpha-4 integrin antibody refers to an antibody that binds to an alpha-4 integrin, such as to the α4 subunit of alpha-integrin, and at least partially inhibits an activity of alpha-4 integrin, particularly a binding activity of an alpha-4 integrin or a signaling activity, e.g., ability to transduce an alpha-4 integrin mediated signal. For example, an alpha-4 integrin binding antibody may inhibit binding of alpha-4 integrin to a cognate ligand of alpha-4 integrin, e.g., a cell surface protein such as VCAM-1, or to an extracellular matrix component, such as fibronectin or osteopontin. An alpha-4 integrin binding antibody may bind to either the α4 subunit or the β1 subunit, or to both. In one embodiment, the antibody binds to the B1 epitope of α4. An alpha-4 integrin binding antibody may bind to alpha-4 integrin with a K_{d} of less than about 10⁻⁶, 10⁻⁷, 10⁻⁸, 10⁻⁹, or 10⁻¹⁰ M. Alpha-4 integrin is also known as VLA-4, alpha4/beta1 and CD29/CD49b. Natalizumab is also known as Antegren7, and AN100226 (see U.S. Pat. No. 8,349,321). Each light chain consists of 213 amino acid residues and each heavy chain consists of 450 amino acid residues. Thus, natalizumab consists of 1326 amino acids and has a total molecular weight of approximately 149 kDa (glycosylated). The term natalizumab is also intended to encompass so-called bio-similar or bio-better variants of the natalizumab protein used in commercially available Tysabri®. For example, a variant of commercial Tysabri® may be acceptable to the FDA when it has essentially the same pharmacological effects as commercially available Tysabri®, even though it may exhibit certain physical properties, such as glycosylation profile, that may be similar but not identical to Tysabri®.

For the purposes of the present application, the term “natalizumab” also encompasses natalizumab with minor modifications in the amino acid structure (including deletions, additions, and/or substitutions of amino acids) or in the glycosylation properties, which do not significantly affect the function of the polypeptide.

The term “antibody”, as used herein, refers to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In one embodiment of the invention, the formulation contains an antibody with CDR1, CDR2, and CDR3 sequences like those described in. U.S. Pat. No. 5,840,299, US2013/0059337, and WO 2011/130603.

The term “meglumine” refers to a compound with chemical formula H₃NHCH₂(CHOH)₄CH₂OH, also known as 1-Deoxy-1-methylaminosorbitol; N-Methyl-d-glucamine; and 1-Deoxy-1-methylamino-D-glucitol.

The terms “mannosylglycerate,” “mannosyllactate,” “mannosylglycolate”, and “diglycerolphosphate” are well known in the art and have their commonly accepted meanings. The following references describe these compounds in some detail: Faria et al., Carbohydrate Res. 2008, 343: 3025-3033; Borges et al., Extremophiles 2002, 6: 209-216; Faria et al., ChemBioChem 2003, 4: 734-741; Sawangwan et al., Biotechnol. J. 2010, 5: 187-191; and Pais et al., J. Mol. Biol. 2009, 394: 237-250. The application incorporates by reference the description of these compounds contained in these references.

The term “sugar” refers to monosaccharides, disaccharides, and polysaccharides. Examples of sugars include, but are not limited to, sucrose, glucose, dextrose, trehalose, lactose, and maltose.

The term “polyol” refers to an alcohol containing multiple hydroxyl groups. Examples of polyols include, but are not limited to, mannitol, sorbitol, glycerol, xylitol and inositol.

The term “metal ion” refers to a metal atom with a net positive or negative electric charge. For the purposes of the present application, the term “metal ion” also includes sources of metal ions, including but not limited to metal salts.

The term “long-term storage” in connection with “formulation” or “pharmaceutical composition” is understood to mean that the formulation or pharmaceutical composition can be stored for three months or more, for six months or more, and preferably for one year or more. Generally speaking, the terms “long term storage” and “long term stability” further include stable storage durations that are at least comparable to or better than the stability of currently available commercial formulations of natalizumab, without losses in stability that would render the formulation unsuitable for its intended pharmaceutical application. Long term storage is also understood to mean that the pharmaceutical composition is stored either as a liquid at 2-8° C., or is frozen, e.g., at −20° C., or colder for storage periods as generally described above. It is also contemplated that the composition can be frozen and thawed more than once. Long term storage stability is also intended to denote the ability of the pharmaceutical natalizumab compositions disclosed herein to resist particulates formation such that the compositions, under long term storage conditions typical of protein therapeutics, exhibits levels and types of particulates that are at least comparable to, and preferably better than commercially available natalizumab formulations. A reduced tendency to form particulates in formulations disclosed herein results in natalizumab formulations having reduced immunogenicity, and therefore reduced potential to cause harm to patients resulting from such immunogenicity.

The term “stable” **with respect to long-term storage is understood to mean that natalizumab contained in the formulation or pharmaceutical compositions does not lose more than 20%, or more preferably 15%, or even more preferably 10%, and most preferably 5% of its activity relative to activity of the formulation or composition at the beginning of storage. The term also should be understood to mean that the natalizumab formulations or compositions are at least comparable to, and preferably better than commercially available natalizumab compositions, in terms of their stability and/or ability to resist formation of particulates during long term storage.

Stability of a protein in an aqueous formulation may also be defined as the percentage of monomer, aggregate, or fragment, or combinations thereof, of the protein in the formulation. A protein “retains its physical stability” in a formulation if it shows substantially no signs of aggregation, precipitation and/or denaturation upon visual examination of color and/or clarity, or as measured by UV light scattering or by size exclusion chromatography. In one aspect of the invention, a stable aqueous formulation is a formulation having less than about 10%, or less than about 5% of the protein being present as aggregate in the formulation

Various analytical techniques for measuring protein stability, including techniques for measuring the type and degree of particulates that may be present in protein formulations, are available in the art and are reviewed in PEPTIDE AND PROTEIN DRUG DELIVERY, 247-301 (Vincent Lee ed., New York, N.Y., 1991) and Jones, 1993 Adv. Drug Delivery Rev. 10: 29-90, for examples. Stability can be measured at a selected temperature for a selected time period, e.g., as described in the examples below.

The term “mammal” includes, but is not limited to, a human.

The term “pharmaceutically acceptable carrier” refers to a non-toxic solid, semisolid or liquid filler, diluent, encapsulating material, formulation auxiliary, or excipient of any conventional type. A pharmaceutically acceptable carrier is non-toxic to recipients at the dosages and concentrations employed and is compatible with other ingredients of the formulation.

The term “pharmaceutical composition” or “formulation” as used herein refers to a mixture of a protein, such as an antibody, e.g., natalizumab, together with one or more additional components. In some embodiments, the additional component is water or a buffer. In other embodiments, the additional components may include, e.g., one or more excipients, such as a stabilizer, tonicity modifier, surfactant, and the like, e.g., a pharmaceutically acceptable carrier or excipient that is conventional in the art and which is suitable for administration to a subject for therapeutic, diagnostic, or prophylactic purposes. For example, pharmaceutical compositions/formulations according to the present invention may be aqueous formulation suitable for injection (or dilution into an i.v. bag for subsequent infusion).

The term “substantially free” of a particular substance means that either the substance is not present or only minimal, trace amounts of the substance are present which do not have any substantial impact on the properties of the composition. If reference is made to no amount of a substance (or that the substance is not present), it should be understood as “no detectable amount”.

By “isotonic” is meant that the formulation of interest has essentially the same osmotic pressure as human blood. Isotonic formulations generally have an osmotic pressure from about 250 to 350 mOsM, although tonicities as high as 1000 mOsM may be tolerated for direct injection into mammals. Isotonicity can be measured using a vapor pressure or ice-freezing type osmometer, for example.

As used herein, “buffer” refers to a buffered solution that resists changes in pH by the action of its acid-base conjugate components. Buffers suitable for use in connection with this invention may have a pH in the range from about 4.0 to about 9.0; preferably from about pH 4.0 to about 7.0; for example, from about pH 4.5 to about 6.5. A pH of any point in between the above ranges is also contemplated.

As used herein, “purified” means that a molecule is present in a sample at a concentration of at least 95% by weight, or at least 98% by weight of the sample in which it is contained.

When pharmaceutical compositions containing natalizumab, including aqueous and lyophilized formulations of natalizumab, are stored on a long-term basis, the activity of natalizumab can be lost or decreased due to aggregation and/or degradation. Thus, the present invention provides aqueous formulations of natalizumab that allow stable long-term storage of natalizumab, so that natalizumab is stable over the course of storage either in liquid or frozen states. The provided formulations do not require any extra steps such as rehydrating.

Numerous embodiments of the present invention are explained in a greater detail below.

### A. Natalizumab

All of the compositions of the present invention comprise natalizumab. As explained in the Background section of this application, natalizumab is a recombinant human IgG4 monoclonal antibody specific for alpha-4 integrin (also called very late antigen-4 or VLA-4). Natalizumab consists of 1326 amino acids and has a molecular weight of approximately 146 kilodaltons (unglycosylated). Natalizumab has been described and claimed in U.S. Pat. No. 5,840,299. The term “natalizumab” is also intended to mean so-called “bio-similar” and “bio-better” versions of the active natalizumab protein present in commercially available Tysabri®. For example, a variant of commercial Tysabri® may be acceptable to the FDA when it has essentially the same pharmacological effects as commercially available Tysabri®, even though it may exhibit certain physical properties, such as glycosylation profile, that may be similar but not identical to Tysabri®.

For the purposes of the present application, the term “natalizumab” also encompasses natalizumab with minor modifications in the amino acid structure (including deletions, additions, and/or substitutions of amino acids) or in the glycosylation properties, which do not significantly affect the function of the polypeptide.

Natalizumab suitable for storage in one of the present pharmaceutical compositions or formulations can be produced by standard methods known in the art. For example, U.S. Pat. No. 5,840,299 and WO2011/130603 describe various methods that a skilled artisan could use to prepare natalizumab protein for use in the formulations of the present invention. These methods are incorporated by reference herein. For example, natalizumab can be prepared by recombinant expression of immunoglobulin light and heavy chain genes in a host cell, e.g., as described in Example 1A. Natalizumab may produced, e.g., in non-immunoglobulin secreting (NS/0) murine myeloma cells, or, e.g., in Chinese Hamster Ovary (CHO) cells. Methods of genetically engineering cells to produce polypeptides are well known in the art. See, e.g., Ausubel et al., eds. (1990), Current Protocols in Molecular Biology (Wiley, New York). Such methods include introducing nucleic acids that encode and allow expression of the polypeptide into living host cells. These host cells can be bacterial cells, fungal cells, or, preferably, animal cells grown in culture. Examples of animal cell lines that can be used are NS/0, CHO, VERO, BHK, HeLa, Cos, MDCK, 293, 3T3, and W138. New animal cell lines can be established using methods well known by those skilled in the art (e.g., by transformation, viral infection, and/or selection). Optionally, natalizumab can be secreted by the host cells into the medium.

Purification of the expressed natalizumab can be performed by standard methods known in the art. When natalizumab is produced intracellularly, the particulate debris is removed, for example, by centrifugation or ultrafiltration. When natalizumab is secreted into the medium, supernatants from such expression systems can be first concentrated using standard polypeptide concentration filters. Protease inhibitors can also be added to inhibit proteolysis and antibiotics can be included to prevent the growth of microorganisms.

For example, natalizumab can be purified using, for example, hydroxyapatite chromatography, gel electrophoresis, dialysis, ion exchange chromatography and affinity chromatography, and any combination of known or yet to be discovered purification techniques, including but not limited to Protein A chromatography, fractionation on an ion-exchange column, hydrophobic interaction chromatography (HIC), ethanol precipitation, reverse phase HPLC, chromatography on silica, chromatography on heparin SEPHAROSET®, an anion or cation exchange resin chromatography (such as a polyaspartic acid column), chromatofocusing, SDS-PAGE, and ammonium sulfate precipitation.

Excipient-free and/or buffer-free natalizumab may also be purified from commercially-available Tysabri® preparations using standard methods, e.g., as detailed in Example 1B, below.

B. pH/Buffer Selection for Natalizumab Formulations

The formulations of the invention may include buffers, tonicity modifiers, excipients, pharmaceutically acceptable carriers and other commonly used inactive ingredients of the pharmaceutical compositions.

Buffers maintain pH in a desired range, e.g., between pH 4 and pH 9. Buffers may also serve to stabilize natalizumab by a variety of other mechanisms, meaning they may be used outside of the nominal buffer capacity range indicated by their respective pKa values. Suitable buffers include acetate (e.g., at pH 4 to 5.5), citrate (e.g., at pH 5 to 6.5), histidine (e.g., at pH 5 to 7), phosphate (e.g., at about pH 6 and 8), Tris (e.g., at pH 7 to 8), and glycine (e.g., at pH 8 to 9). Specific embodiments include, without limitation, sodium or potassium phosphate, sodium or potassium citrate, ammonium acetate, tris-(hydroxymethyl)-aminomethane (tris), various forms of acetate and diethanolamine. Other suitable buffers include succinate, histidine, tartrate, bicarbonate, borate, and maleate. The concentration of the buffer in the formulation is preferably between about 1 mM to about 1M, and more preferably about 10 mM to about 200 mM. Buffers are well known in the art and are manufactured by known methods and available from commercial suppliers. The selection of an appropriate buffer may be informed by data on any interactions of specific buffers with other formulation components. For example, U.S. Pat. No. 8,349,321 discloses that degradation of polysorbate 80 (thought to be due to a metal catalyzed oxidation of polysorbate) is significantly impeded by replacing histidine buffer with phosphate buffer in a tested placebo formulation.

The pH of the pharmaceutical compositions of the invention is generally between pH 4 and pH 8. Preferably the pH of the pharmaceutical compositions is between about 4 and about 7; in specific embodiments it is between about 4.5 and about 6.5. A person of ordinary skill in the art will understand that the pH can be adjusted as necessary to maximize stability and solubility of natalizumab in a particular formulation.

### C. Excipients Suitable for Use in Natalizumab Formulations

Excipients include components of a pharmaceutical formulation other than the active ingredient and are typically added during formulation development for a specific purpose, e.g., to confer favorable properties to the formulation, e.g., stabilize the polypeptide while in solution (also in dried or frozen forms), etc. Excipients are well known in the art and are manufactured by known methods and available from commercial suppliers. Excipients may include, for example, tonicity modifiers, stabilizers, salts, chelating agents, sacrificial additives and surfactants, and miscellaneous excipients such as ammonium sulfate, magnesium sulfate, sodium sulfate, trimethylamine N-oxide, betaine, metal ions (e.g., zinc, copper, calcium, manganese, and magnesium), CHAPS, monolaurate, 2-O-beta-mannoglycerate, and the like. The concentration of one or more excipients in a formulation of the invention is/are preferably between about 0.001 to 30 weight percent, more preferably about 0.01 to 10 weight percent.

I. Tonicity Modifiers

A tonicity modifiers any molecule that contributes to the osmolality of a solution. Note that the tonicity modifier may also provide some degree of conformational or colloidal stabilization as well. The osmolality of a pharmaceutical composition is preferably adjusted to maximize the active ingredient's stability and/or to minimize discomfort to the patient upon administration. It is generally preferred that a pharmaceutical composition for direct administration to a patient be isotonic with serum, i.e., having the same or similar osmolality, which is achieved by addition of a tonicity modifier. However, hypertonic formulations which would then be diluted in an isotonic vehicle are also within the scope of this invention.

In one embodiment, the osmolality of the provided formulations is from about 180 to about 500 mOsM, more preferably between 250 and 350 mOsM. However, it is to be understood that the osmolality can be either higher or lower as specific conditions require.

Examples of tonicity modifiers suitable for modifying osmolality include, but are not limited to amino acids (e.g., cysteine, arginine, histidine and glycine), salts (e.g., sodium chloride, potassium chloride and sodium citrate) and/or nonelectrolytes (e.g., sugars or polyols, such as, for example, sucrose, glucose and mannitol).

In one embodiment, the concentration of the tonicity modifier in the formulation is preferably between about 1 mM to about 1 M, more preferably about 50 mM to about 500 mM. Tonicity modifiers are well known in the art and are manufactured by known methods and available from commercial suppliers.

II. Stabilizers

Stabilizers are a class of excipients that include sugars, polymers, and polyols as well as amino acids, which provide some degree of conformational stability. They may also improve chemical as well as physical stability of a protein. Specific examples suitable for use with the invention (in the context of optimized pH/buffer systems as described above) include sucrose, dextrose, maltose, lactose, raffinose, and trehalose; sorbitol, maltitol, xylitol, and mannitol may also be employed for this purpose. Additional stabilizers suitable for use with the invention include amino acids, such as (but not limited to) glycine, arginine, glutamate, and proline (either as the free base form or as a salt form). The invention further includes specific combinations of amino acids, e.g., Arg and Glu, as well as specific combinations of one or more amino acids with one or more polyols (e.g., Gly together with sorbitol or mannitol), which have particularly desirable properties.

a. Natalizumab Formulations Stabilized with Sugars and/or Polyols

In one embodiment, the invention provides a stable aqueous formulation comprising natalizumab, a sugar and/or a polyol; and optionally an amino acid. Examples of sugars include sucrose, lactose, maltose, trehalose, and glucose. Examples of polyols include glycerol, sorbitol, mannitol, xylitol, and maltitol.

Experiments in accordance with Example 3A may be performed to determine the effects of a combination of a sugar and/or a polyol (and optionally an amino acid) on the physical stability and other properties of specific natalizumab formulations, e.g., to assess such stabilizers for their effects on any tendency of natalizumab to associate in an undesirable conformation, and therefore on any aggregation in natalizumab formulations. Any improvements in formulation properties, e.g., a reduction in aggregation, due to such stabilizers may last for extended periods, e.g., 6 months, 9 months, a year, or up to two years or more. Thus, a combination of a sugar and/or a polyol (and optionally an amino acid) may stabilize aqueous pharmaceutical compositions containing natalizumab.

Without wishing to be bound to a particular theory, the combination of a sugar and/or a polyol (and optionally an amino acid) may be synergistic for the purposes of stabilizing natalizumab because even though excluded solutes are, on average, residing in the bulk, rather than on the surface of the protein, there may be interactions between sugars/polyols and the protein. Those interactions may differ between sugars and smaller polyols or amino acids. In addition, at high concentrations, the two additives may alter the thermodynamic activity of the other, thereby leading to solution behavior that may be different than what would be observed for each individual component.

The pharmaceutical compositions of the invention may be prepared by combining, a purified natalizumab, a sugar, and/or a polyol (and optionally amino acid). Further, a buffer, a tonicity modifier and an additional excipient may be added as needed. A person of ordinary skill in the art will understand that the combining of the various components to be included in the composition can be done in any appropriate order. For example, the buffer can be added first, middle or last, and the tonicity modifier can also be added first, middle or last. A person of ordinary skill in the art will also understand that some of these chemicals can be incompatible in certain combinations, and accordingly, are easily substituted with different chemicals that have similar properties but are compatible in the relevant mixture.

In some embodiments, a sugar and a polyol may act in concert. For example, amino acids, such as proline, serine, or glutamate maybe used together with a sugar to achieve a stability profile better than either excipient could provide on its own. In one embodiment, the ratio of a sugar to a polyol (or amino acid) in the formulation is between 5:1 and 1:5.

Suitable sugars include without limitation sucrose, trehalose, lactose, raffinose, and maltose. Suitable polyols include without limitation sorbitol, mannitol, glycerol, and propylene glycol. Suitable amino acids include without limitation glycine, alanine, glutamate, proline, serine, and threonine. Sugars, polyols and amino acids are available from commercial suppliers

In one embodiment, the concentration of a sugar in the provided formulations is between about 0.1% (w/v) to 40%, for example about 1% to about 20%, about 2% to about 10%, or about 5% to 9%.

In one embodiment, the concentration of a polyol in the provided formulations is between about 0.1% to 30%, for example about 1% to about 10%, or about 2% to about 5%.

In one embodiment, a formulation of the invention comprises about 10 to about 200 mg/ml of natalizumab; about 10 mM to about 350 mM sucrose; about 0 mM to about 100 mM mannitol; about 5 mM to about 50 mM buffer; and about 0 mM to about 200 mM NaCl, at about pH 5 to about pH 7.

In another embodiment, sucrose can be replaced with another sugar such as trehalose (at about 10 mM to about 350 mM) in the formulation. In yet another embodiment, mannitol can be replaced with another polyol such as sorbitol (at about 0 mM to about 100 mM) in the formulation.

The formulations of the invention may also include buffering agents, tonicity modifiers, excipients, and other commonly used inactive ingredients of the pharmaceutical compositions.

b. Natalizumab Stabilized with Amino Acids

Amino acids, e.g., proline, serine, sodium glutamic acid, glutamate, alanine, histidine, tryptophan, tyrosine, arginine, glycine, lysine, methionine, proline, glutamic acid, aspartic acid, sarcosine, glycine betaine, and mixtures of the foregoing may be employed as stabilizers in certain natalizumab formulations. These compositions may use the free base form of the amino acid or any conjugate acid form such as a hydrochloride salt. Such amino acids are readily available from commercial suppliers. For example, in one embodiment, the invention provides a stable aqueous pharmaceutical composition comprising natalizumab and one or more amino acids, wherein the amino acid(s) is selected from the group consisting of serine, proline, glycine, alanine, glutamate, arginine and combinations thereof. The composition may further optionally include a sugar and/or polyol.

Experiments such as those described in Example 3, may be performed to assess the effects of amino acids such as serine, glycine, alanine, glutamate and/or arginine, on the stability & other properties of specific natalizumab formulations, e.g., any tendency for natalizumab to associate in undesired ternary or quaternary complexes. Such additives may improve the properties of the natalizumab formulations, e.g., by reducing aggregation. Any such improvement in properties, e.g., reduction in aggregation, may last for extended periods, e.g., 6 months, 9 months, a year, or up to two years or more. Without wishing to be bound to a particular theory, it is believed that amino acids such as serine, proline and glutamate are able to stabilize aqueous pharmaceutical compositions containing natalizumab because they are excluded from the surface of the protein, resulting in net conformation stabilization.

The formulations described above may be prepared, e.g., by combining, a purified natalizumab and one or more of the above-referenced amino acids. Further, a buffer, a tonicity modifier and an additional excipient can be added as needed.

In one embodiment, the concentration of the amino acids in the provided formulations is between about 1 mM and about 500 mM. In another embodiment, the concentration of the amino acid(s) is between about 10 mM and about 350 mM; in related embodiments, the concentration of the amino acid(s) is about 50 mM, 100 mM, 150 mM, 200 mM, 220 mM, 240 mM, 260 mM, 280 mM, 300 mM, 320 mM and 340 mM, for example, 50-100 mM, 100-150 mM, 150-200 mM, 200-300 mM, 200-250 mM, 250-300 mM, and 300-350 mM.

III. Salts

Salts, e.g., NaCl, are often part of protein (e.g., antibody) formulations. For instance, salts such as NaCl, Na2SO4, and KCl, together with specific buffer combinations as described above, can confer particularly advantageous stability properties. Such salts may be used with as well as without a polyol added (e.g., mannitol and NaCl). For example, concentrations as low as 20 to 50 mM can provide decreased levels of proteolysis (such as hinge region hydrolysis), oxidation, deamidation, or other chemical instabilities. Furthermore, low levels of salt (<50 mM) may lead to improved colloidal stability. Likewise, it is known that high concentrations of certain salts (up to 400 mM or more) can increase conformational stability. The specific effects of NaCl and other salts on natalizumab is dependent on pH, buffer composition, and stress condition. Experiments performed in support of the present invention demonstrate that the use of intermediate levels of salt (e.g., 50-200 mM) is beneficial in certain formulations.

Replacing NaCl with Other Salts. In specific embodiments, sodium chloride is replaced with Na₂SO₄, KCl, MgCl₂, CaCl₂, MgSO₄, ZnCl₂, or other physiologically-acceptable salts, e.g., to reduce or eliminate the NaCl load in the formulation. In a more specific embodiment, such replacement is particularly advantageous in cases where the buffer is not a phosphate buffer.

IV. Surfactants

Surfactants can confer protection against agitation and freeze/thaw damage, as well as stabilizing a formulation during storage. As further detailed in Example 4, surfactants which may be employed with natalizumab formulations of the present invention include Tween®-80 (polysorbate 80, PS 80), Tween®-20 (polysorbate 20, PS 20), SDS, polysorbate, polyoxyethylene copolymer, Brij 35, Triton X-10, poloxamer 188 (Pluronic F-68), Pluronic F127, and Maltosides, e.g., n-Decyl-β-D-maltopyranoside (DM), n-Dodecyl-β-D-maltopyranoside (DDM), and 6-Cyclohexyl-1-hexyl-β-D-maltopyranoside (Cymal-6). In certain embodiments, surfactants that may be particularly advantageous include PS 20, PS 40, PS 60, and PS 80 at different concentrations, as well as DMM and poloxamer 188 (Pluronic F-68). Other zwitterionic or nonionic surfactants may be used as well.

As above, optimal formulation employing such surfactants may also employ specific buffers at specific pH ranges, and may optionally include other tonicity modifier such as NaCl, polyol, or amino acid(s).

V. Polymers

Polymers, such as dextrans, starches (e.g., hydroxyl ethyl starch (HETA)), poly(ethylene glycols (PEGs), e.g., PEG-3350 or PEG-4000, may also provide stabilization to natalizumab, presumably by being excluded from the surface of the protein due to steric effects arising from their higher molecular weight. In particular, hydrophilic polymers, such as polyethylene glycols (PEGs), polysaccharides, and inert proteins, and may be used to stabilize proteins and enhance protein assembly. Examples include dextran, hydroxyl ethyl starch (HETA), PEG-4000, and gelatin. Additionally, non-polar moieties on certain polymers such as PEGs and Pluronics can decrease water surface tension rendering them as surfactants that suppress surface adsorption induced aggregation. In one embodiment, the concentration of polymer is between 0.01% and 40%, more preferably between 1 and 15%. The formulations of the invention may include combination of polymers with sugars, polyols, or amino acids in any combination.

Under certain conditions, natalizumab may be stable even in the absence of surfactants, and/or may be stabilized with surfactants other than PS80 and at lower surfactant concentrations. For example, certain polymers, e.g., PEG, can exhibit surfactant-like properties and may be employed to stabilize natalizumab formulations in the absence of surfactants according to the present invention. Additional polymers which may be employed in specific natalizumab formulations include serum albumin (bovine serum albumin (BSA), human SA or recombinant HA), dextrans, poly(vinyl alcohol (PVA), hydroxypropyl methylcellulose (HPMC), polyethyleneimine, gelatin, polyvinylpyrrolidone (PVP), hydroxyethylcellulose (HEC), and 2-Hydroxypropyl-beta-cyclodextrin (HP-beta-CD).

VI. Chelating Agents

Chelating Agents such as EDTA, DPTA, etc., and/or sacrificial additives (e.g., ascorbate, Met), may be employed at specific pH values and with and without buffers (that may also acts as chelating agents, e.g., citrate, phosphate) to enhance the formulation properties, especially in cases where there may be some level of oxidative damage (under certain conditions, certain metals can catalyze the degradation of antibodies, especially at the hinge region). The addition of a chelating agent, such EDTA and DPTA, may be beneficial at improving the storage stability of natalizumab. Such approaches may be employed to stabilize natalizumab formulations according to the present invention.

The inclusion of chelating agents, such as EDTA, reduces the levels of metal-catalyzed oxidation for natalizumab and also decreases metal-catalyzed hydrolysis of the hinge region. Certain buffers, such as citrate, may also function as chelating agents and can serve multiple purposes in stabilization of natalizumab.

Sacrificial additives are well known to diminish certain oxidation events, such as oxidation of methionine residues. Addition of the free amino acid, methionine, or some derivative, can lead to decreased oxidation of natalizumab. Ascorbate and various thiol derivatives can serve the same purpose. Likewise, Trp and its derivatives can also serve as a sacrificial additive, even in the case of photolytic oxidation.

### D. Natalizumab Formulations

I. Formulations of Natalizumab Using a Single Buffer System

In other embodiments, the invention provides a stable aqueous pharmaceutical composition comprising natalizumab, a polyol, a surfactant, and a buffer system comprising a single buffering agent, the single buffering agent being selected from citrate, phosphate, succinate, histidine, tartrate or maleate, but not including combinations of the foregoing; wherein the formulation has a pH of about 4 to 8, e.g., about 4 to about 7. Histidine, acetate, and citrate are particularly suitable for use as single buffering agents. Note that the buffer may not need to possess significant buffering capacity to provide stabilization of natalizumab at a particular pH. In the single buffer embodiment, natalizumab can be present at a concentration from about 10 to about 200 mg/ml, from about 20 to about 150 mg/ml, etc. The buffer is present at a concentration from about 5 mM to about 50 mM. The pH of the compositions is between about 4 and about 8, preferably between about 4.5 and 6.5. The single buffer compositions of the invention may further comprise a stabilizer selected from the group consisting of an amino acid, a salt, a chelating agent and a metal ion. The amino acid is selected from the group consisting of glycine, alanine, glutamate, arginine and methionine, preferably from glycine, arginine and methionine. The salt is selected from the group consisting of sodium chloride and sodium sulfate. The metal ion is selected from the group consisting of zinc, magnesium and calcium. The compositions of the invention may further comprise a surfactant. The surfactant can be a polysorbate surfactant or a poloxamer. Polysorbate surfactants include polysorbate 80, polysorbate 40 and polysorbate 20. Poloxamer surfactants include poloxamer 188 (also available commercially as Pluronic F-68). The single buffer composition may further comprise a polyol such as a sugar alcohol, e.g., mannitol or sorbitol. The single buffer natalizumab composition may also comprise a sugar, e.g., sucrose, trehalose or dextrose.

In one embodiment of a single buffer natalizumab formulation, the invention provides a stable aqueous pharmaceutical composition comprising natalizumab at a concentration from about 20 and about 150 mg/ml, polysorbate 80 at a concentration from about 0.001% to 0.1%, and succinate at a concentration from about 5 mM and about 100 mM, wherein the composition has a pH of about 4 to about 7, and wherein the composition is substantially free of any other buffers.

In one embodiment of a single buffer natalizumab formulation, the invention provides a stable aqueous pharmaceutical composition comprising natalizumab at a concentration from about 20 and about 150 mg/ml, polysorbate 80 at a concentration from about 0.001% to 0.1%, and citrate at a concentration from about 5 mM and about 100 mM, wherein the composition has a pH of about 4 to about 7, and wherein the composition is substantially free of any other buffers.

In another embodiment of a single buffer natalizumab formulation, the invention provides a stable aqueous pharmaceutical composition comprising natalizumab at a concentration from about 20 and about 150 mg/ml, polysorbate 80 at a concentration from about 0.001% to 0.1%, and histidine at a concentration from about 5 mM and about 100 mM, wherein the composition has a pH of about 4 to about 7, and wherein the composition is substantially free of any other buffers.

In a further embodiment of a single buffer natalizumab formulation, the invention provides a stable aqueous pharmaceutical composition comprising natalizumab at a concentration from about 20 and about 150 mg/ml, polysorbate 80 at a concentration from about 0.001% to 0.1%, and either tartrate, maleate or acetate at a concentration from about 5 mM and about 100 mM, wherein the composition has a pH of about 4 to about 7, and wherein the composition is substantially free of any other buffers.

II. Formulations of Natalizumab which Exclude Buffer

In a further embodiment, the invention provides a stable aqueous pharmaceutical composition comprising natalizumab, and optionally a stabilizer comprising at least one member selected from a polyol and a surfactant, wherein the composition has a pH of about 4 to about 8, e.g., about 4 to about 7, and wherein the composition is substantially free of a buffer. The term “free of buffer” should be understood to allow inclusion of the inherent buffering effect of the protein itself. In a buffer free formulation, the stabilizers referenced above may also be present (e.g. glycine, arginine and combinations thereof). Such “self-buffering” or “buffer-free” protein formulations comprise a protein, e.g., a pharmaceutical protein, and are buffered by the protein itself, i.e., the formulations do not require additional buffering agents to maintain a desired pH. The protein (preferably at a concentration of 20 mg/mL or more) is substantially the only buffering agent in such formulations (i.e., other ingredients, if any, do not act substantially as buffering agents in the formulation). See, e.g., Gokarn, et al., US 2008/0311078.

III. Formulations of Natalizumab which Exclude Surfactant

In a further embodiment, the invention provides a stable aqueous pharmaceutical composition comprising natalizumab, a polyol, and a buffer selected from the group consisting of acetate, citrate, phosphate, succinate, histidine, tartrate and maleate, wherein the composition has a pH of about 4 to about 8, e.g., about 4 to about 7, and wherein the composition is free or substantially free of a surfactant. In one embodiment, the composition (i) the buffer is at least one member selected from the group consisting of histidine, acetate, citrate, and succinate; and (ii) the polyol is not mannitol at concentrations less than about 150 mM, but instead is selected from the group consisting of mannitol at concentrations exceeding about 150 mM, sorbitol and trehalose.

IV. Formulations of Natalizumab which Exclude Polyol

In a further embodiment, the invention provides a stable aqueous pharmaceutical composition comprising natalizumab, a surfactant, and a buffer selected from the group consisting of acetate, citrate, phosphate, succinate, histidine, tartrate and maleate, wherein the composition has a pH of about 4 to about 8, and about 4 to about 7, and wherein the composition is substantially free of polyol. In one embodiment, the buffer is at least one member selected from the group consisting of histidine and succinate.

### Additional Stabilizers Useful in Embodiments I Through IV

Optionally, in each of the embodiments summarized above, the composition may further comprise a stabilizer selected from the group consisting of an amino acid, a salt, a chelating agent and a metal ion. The amino acid stabilizer may be selected from the group consisting of glycine, alanine, serine, glutamate, arginine and methionine. The salt stabilizer may be selected from the group consisting of sodium chloride and sodium sulfate. The metal ion stabilizer may be selected from the group consisting of zinc, magnesium and calcium.

In an embodiment, (i) the optional additional stabilizer present in this embodiment is not sodium chloride, and comprises at least one or both of arginine and glycine; (ii) the buffer, when present, is selected from at least one of acetate, phosphate, citrate, tartrate or maleate, histidine and succinate; and (iii) the stabilizer when it includes a polyol is not mannitol unless in amounts greater than about 150 mM, and may also include trehalose and sorbitol. Preferably the amount of mannitol is greater than about 150 mM, and most preferably greater than about 200 mM.

V. Formulations of Natalizumab that Exclude Both Surfactant and Polyol

It has been further discovered that satisfactory stabilization can be attained when the stabilizers mentioned above are used in place of both polyol and surfactant, accordingly, in a further embodiment, the invention provides a stable aqueous pharmaceutical composition comprising natalizumab, optionally a buffer, a stabilizer selected from the group consisting of an amino acid, a salt, EDTA, and a metal ion, and wherein the composition has a pH of about 4 to about 8, e.g., about 4 to about 7, and wherein the composition is free or substantially free of a both polyol and surfactant. This embodiment includes formulations that exclude buffer, polyol and surfactant, including such formulations that comprise about 1.5 to about 3 times the concentration of natalizumab found in commercially available formulations of the protein.

In an example of Embodiment I and II, above, the invention provides a stable aqueous pharmaceutical composition comprising natalizumab at a concentration from about 20 and about 150 mg/ml, sorbitol or trehalose at a concentration from about 1 to 10% weight by volume, polysorbate at a concentration from about 0.001% to 0.1%, and at least one of acetate, succinate, histidine, phosphate, tartrate, maleate or citrate buffer, at a concentration from about 5 mM to about 100 mM, wherein the composition has a pH of about 4 to about 7.

In an example of Embodiment IV, the invention provides a stable aqueous pharmaceutical composition comprising natalizumab at a concentration from about 20 and about 150 mg/ml, sorbitol or trehalose at a concentration from about 1 to 20% weight by volume, and at least one of acetate, succinate, histidine, phosphate, tartrate, maleate or citrate buffer, at a concentration from about 5 mM to about 100 mM, wherein the composition has a pH of about 4 to about 7, and wherein the composition is substantially free of a surfactant.

In an example of Embodiment V, the invention provides a stable aqueous pharmaceutical composition comprising natalizumab at a concentration from about 20 and about 150 mg/ml, glycine at a concentration from about 20 to about 200 mM, and at least one of acetate, succinate, histidine, phosphate, tartrate, maleate or citrate buffer, at a concentration from about 5 mM to about 100 mM, wherein the composition has a pH of about 4 to about 7, and wherein the composition is free or substantially free polyol; surfactant (e.g. PS80) is preferably, but optionally present.

In a further example of Embodiment V, the invention provides a stable aqueous pharmaceutical composition comprising natalizumab at a concentration from about 20 and about 150 mg/ml, arginine or glycine at a concentration from about 1 to about 250 mM, and at least one of acetate, succinate, histidine, phosphate, tartrate, maleate or citrate buffer, at a concentration from about 5 mM and about 100 mM wherein the composition has a pH of about 4 to about 7, and wherein the composition is substantially free of polyol. Surfactant (e.g. PS80) is preferably but optionally present, and the composition is, optionally, free or substantially free of citrate/phosphate buffer combination.

In a further example of Embodiment V, the invention provides a stable aqueous pharmaceutical composition comprising natalizumab at a concentration from about 20 and about 150 mg/ml, sodium chloride at a concentration from about 4 to about 200 mM, and at least one of acetate, succinate, histidine, phosphate, tartrate, maleate or citrate buffer, at a concentration from about 5 mM and about 100 mM wherein the composition has a pH of about 4 to about 7, and wherein the composition is free or substantially free of a polyol. Surfactant (e.g. PS80) is preferably but optionally present.

In an example of Embodiment IV, the invention provides a stable aqueous pharmaceutical composition comprising natalizumab at a concentration from about 20 and about 150 mg/ml, sodium chloride at a concentration from about 5 to about 200 mM, a polysorbate at a concentration from about 0.001% to 0.1%, and at least one of acetate, succinate, histidine, phosphate, tartrate, maleate or citrate buffer, at a concentration from about 5 mM and about 100 mM wherein the composition has a pH of about 4 to about 7, and wherein the composition is free or substantially free of a polyol and, optionally, free or substantially free of citrate/phosphate buffer.

In an example of Embodiments I and II, with additional stabilization, the invention provides a stable aqueous pharmaceutical composition comprising natalizumab at a concentration from about 20 and about 150 mg/ml, polysorbate 80 at a concentration from about 1 to about 50 μM, sorbitol or trehalose at a concentration from about 1 to about 20% weight by volume, a chelating agent at a concentration from about 0.01% to about 0.5%, and at least one of acetate, succinate, histidine, phosphate, tartrate, maleate or citrate, as a sole buffer, at a concentration from about 5 mM and about 100 mM wherein the composition has a pH of about 4 to about 7.

In a further example of Embodiments I and II, with additional stabilization, the invention provides a stable aqueous pharmaceutical composition comprising natalizumab at a concentration from about 20 and about 150 mg/ml, a polysorbate at a concentration from about 0.001% to 0.1%, sorbitol or trehalose at a concentration from about 1 to about 20% weight by volume, methionine at a concentration from about 1 to about 10 mg/ml, and at least one of acetate, succinate, histidine, phosphate, tartrate, maleate or citrate at a concentration from about 5 mM and about 100 mM wherein the composition has a pH of about 4 to about 7.

In a further example of Embodiments I and II, with additional amino acid stabilization, the invention provides a stable aqueous pharmaceutical composition comprising natalizumab at a concentration from about 20 and about 150 mg/ml, a polysorbate at a concentration from about 0.001% to 0.1%, mannitol, sorbitol or trehalose (preferably sorbitol) at a concentration from about 1 to about 20% weight by volume, and an amino acid that is preferably one and not both of (a) arginine at a concentration from about 1 to about 250 mg/ml, and (b) glycine at a concentration of about 20 to 200 mg/ml, and histidine buffer or succinate buffer at a concentration from about 5 mM and about 100 mM, and wherein the composition has a pH of about 4 to about 7.

In a further example of Embodiment III, with additional amino acid stabilization, the invention provides a stable aqueous pharmaceutical composition comprising natalizumab at a concentration from about 20 and about 150 mg/ml, a polysorbate at a concentration from about 0.001% to 0.1%, arginine at a concentration from about 1 to about 250 mg/ml, glycine at a concentration of about 20 to 200 mg/ml, and histidine, citrate, acetate, or succinate buffer at a concentration from about 5 mM to about 100 mM, and wherein the composition has a pH of about 4 to about 7, and is free or substantially free of polyol.

### E. Analytical Methods

In the Examples below, the chemical and physical stability of the natalizumab protein is measured using, e.g., SEC, RP, UV, pH, CE-IEF and CE-SDS. However, other analytical methods may also be employed, for example, biophysical techniques such as those described by Jiskoot and Crommelin (Methods for Structural Analysis of Protein Pharmaceuticals, Springer, New York, 2005). Specific examples of such techniques include spectroscopic analyses (e.g., second derivative ultraviolet spectroscopy, circular dichroism, Fourier Transform infrared spectroscopy, Raman spectroscopy, fluorescence and phosphorescence spectroscopy), thermal analyses (e.g., differential scanning calorimetry), and size based analyses (e.g., analytical ultracentrifuge, light scattering).

One of skill in the art can readily determine which of these or other suitable techniques may be used in specific situations when assessing the physical characteristics (e.g., stability, aggregation, oxidation, etc.) of the natalizumab protein in particular formulations.

### F. Methods of Treatment

Formulations of the present invention may be used in methods of treating a mammal, e.g., a human patient, comprising administering a therapeutically effective amount of the pharmaceutical compositions of the invention to the mammal, e.g., a human patient, wherein the mammal, e.g., a human patient, has a disease or disorder that can be beneficially treated with natalizumab.

For example, natalizumab may be administered for the treatment of patients with relapsing forms of multiple sclerosis, e.g., to delay the accumulation of physical disability and/or reduce the frequency of clinical exacerbations.

Natalizumab may also be administered to induce and maintain clinical response and remission in adult patients with moderately to severely active Crohn's disease with evidence of inflammation who have had an inadequate response to, or are unable to tolerate, conventional CD therapies and inhibitors of TNF-α.

Natalizumab may be administered for multiple sclerosis via a 300 mg intravenous infusion over one hour every four weeks.

The natalizumab used in any of the above treatments is a humanized monoclonal antibody against the cell adhesion molecule α4-integrin.

The provided pharmaceutical compositions may be administered to a subject in need of treatment by injection systemically, such as by intravenous injection; or by injection or application to the relevant site, such as by direct injection, or direct application to the site when the site is exposed in surgery; or by topical application.

Antibodies are typically administered to a subject (e.g., a human) at a concentration of about 0.01 mg/mL to about 200 mg/mL. More typically, antibodies range in concentration from about 0.1 mg/mL to about 150 mg/mL. However, instances exist when greater concentrations are required to be administered to a patient, e.g., about 15 to about 200 mg/mL, about 15 mg/mL to 150 mg/mL, about 20 to about 50 mg/mL, and about 20 mg/mL and any integer value in between.

In certain embodiments, the pharmaceutical formulations of the invention may be prepared in a bulk formulation, and as such, the components of the pharmaceutical composition are adjusted to be higher than would be required for administration and diluted appropriately prior to administration. For example, natalizumab may be supplied as a solution (300 mg per 15 mL vial, i.e., 20 mg/mL) for dilution prior to administration, e.g., intravenous infusion. To prepare the final dosage solution, 15 mL of natalizumab concentrate is withdrawn from the vial using a sterile needle and syringe. The concentrate is injected into 100 mL 0.9% Sodium Chloride Injection, USP. No other IV diluents are necessary to prepare the natalizumab final dosage solution. The final dosage solution is gently inverted to mix completely, but is preferably not shaken. The solution is preferably visually inspected for particulate material prior to administration. The final dosage solution (after dilution) preferably has a concentration of about 2.6 mg/mL.

In another embodiment, an acceptable dose for administration by injection may contain 20-500 mg/dose, or alternatively, containing 300 mg per dose. The dose can be administered weekly, biweekly, or separated by several weeks. In a related embodiment, natalizumab is administered at 300 mg by intravenous infusion. In another embodiment, natalizumab is administered at 300 mg by a single subcutaneous (SC) injection.

In some instances, an improvement in a patient's condition will be obtained by administering a dose of up to about 20 mg/mL of the pharmaceutical composition. Treatment for longer periods may be necessary to induce the desired degree of improvement. For incurable chronic conditions the regimen may be continued indefinitely. For pediatric patients (ages 4-17), a suitable regimen may involve administering a dose of 0.4 mg/kg to 5 mg/kg of natalizumab every four weeks, or 300 mg every four weeks.

The pharmaceutical compositions may be administered as a sole therapeutic or in combination with additional therapies as needed. Thus, in one embodiment, the provided methods of treatment and/or prevention are used in combination with administering a therapeutically effective amount of another active agent. The other active agent may be administered before, during, or after administering the pharmaceutical compositions of the present invention. Another active agent may be administered either as a part of the provided compositions, or alternatively, as a separate formulation.

Administration of the provided pharmaceutical compositions can be achieved in various ways, including parenteral, oral, buccal, nasal, rectal, intraperitoneal, intradermal, transdermal, subcutaneous, intravenous, intra-arterial, intracardiac, intraventricular, intracranial, intratracheal, intrathecal administration, intramuscular injection, intravitreous injection, and topical application.

The pharmaceutical compositions may, if desired, be presented in a vial, pack or dispenser device which may contain one or more unit dosage forms containing the active ingredient. In one embodiment the dispenser device can comprise a syringe having a single dose of the liquid formulation ready for injection. The syringe can be accompanied by instructions for administration.

In another embodiment, the present invention is directed to a kit or container, which contains an aqueous pharmaceutical composition of the invention. The concentration of the polypeptide in the aqueous pharmaceutical composition can vary over a wide range, but is generally within the range of from about 0.05 to about 20,000 micrograms per milliliter (μg/ml) of aqueous formulation. The kit can also be accompanied by instructions for use.

The present invention is described in further detain in the following examples which are not in any way intended to limit the scope of the invention as claimed. The attached FIGURES are meant to be considered as integral parts of the specification and description of the invention. All references cited are herein specifically incorporated by reference for all that is described therein. The following examples are offered to illustrate, but not to limit the claimed invention.

### EXAMPLES

A number of different natalizumab formulations were prepared as described more fully below. The formulations were assessed in blocks of experiments, where each block is described in the context of an Example or portion of an Example. The formulations were exposed to different storage conditions, for example, two weeks at 40° C., four weeks at 25° C., and 13 weeks at 5° C. For each time point, the chemical and/or physical stability of the natalizumab protein was measured by one or more of Ultra-Violet (UV) Absorbance Spectroscopy, Size Exclusion Chromatography (SEC), Reversed Phase High-Performance Liquid Chromatography (RP HPLC), Hydrophobic Interaction Chromatography (HIC), Cation Exchange Chromatography (CEX), and Capillary Electrophoresis Sodium Dodecyl Sulfate (CE-SDS) Analyses.

Unless otherwise indicated, the following abbreviations apply: eq (equivalents); M (Molar); μM (micromolar); N (Normal); mol (moles); mmol (millimoles); μmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds).

### Materials and Methods

Unless otherwise indicated, chemicals and other reagents were sourced from commercial suppliers, including EMD Millipore (Billerica, Mass.), J. T. Baker (Avantor Performance Materials, Center Valley, Pa.), Pfansteichl (Waukegan, Ill.), Sigma-Aldrich (St. Louis, Mo.), and Spectrum (New Brunswick, N.J.). Slide-A-Lyzer™ Dialysis Cassettes (10K cut off) were obtained from Thermo Fisher Scientific (Waltham, Mass.); Ultrafiltration Discs (PLTK04310, Ultracel regenerated cellulose, 30 kDa NMWL, 44.5 mm) were obtained from EMD Millipore (Billerica, Mass.); 1 mL Fiolax clear vials were obtained from Schott (Elmsford, N.Y.).

HPLC Columns used in these studies included the following: Proteomic SCX-NP5 4.6×250 mm 5 μm, Proteomix RP-1000, 5 μm 4.6×100 mm, and Proteomix HIC Butyl-NP5 4.6×100 mm, 5 μm (all from Sepax Technologies, Newark, Del.); and Acquity BEH-200 SEC, 1.7 μm 4.6×300 mm (Waters Corporation, Milford, Mass.).

Equipment used in these studies included the following: a Sartorius Balance (CPA124S); pH meters from Denver Instruments (model 250) and Mettler Toledo (Seven Excellence); a conductivity meter from Mettler Toledo (Seven Excellence); a UV spectrophotometer from Cary (100 Bio); HPLC Dionex 5, Dionex 6, Dionex 7, and Dionex 8 Ultimate 3000 UPLC; Capillary Electrophoresis from Beckman Coulter (P/ACE MDQ); and a Labnet Rocker Plate (P4).

A. Freeze-Thaw Conditions

Freeze thaw samples were typically prepared on the day of analysis to match with t=0. The samples were frozen at −80° C. between 3 to 7 minutes. The frozen sample was then thawed at room temperature until all the ice had thawed. The freeze and thaw cycle was repeated 5 times for each sample.

Block 1 samples underwent freeze thawing stress as follows: the samples were frozen for up to 10 hours and then thawed for 6 to 8 hours; this was repeated five times. After the 5th freeze thaw cycle, the samples were removed for analysis.

Block 2 and 3 samples underwent freeze thawing stress as follows: the samples were frozen for up to 10 hours and then thawed for 6 to 8 hours; this was repeated three times. After the third free thaw cycle, the samples were removed for analysis. After the 4th freeze the samples were stored at −20 C.° for one to two months followed by a 5th freeze thaw.

Block 4 samples underwent the same freeze thawing stress as block 2 and 3 samples. The samples were frozen for up to 10 hours and then thawed for 6 to 8 hours this was repeated three times. After the 3rd freeze thaw cycle samples were removed for analysis. After the 4th freeze the samples were stored at −20 C.° and at −40 C.° for 3 months followed by a 5th freeze thaw.

B. Agitation Studies

Samples were agitated at 250 rpm for 48 hours at 25° C. on a rockerplate. A control was prepared and placed next to the rocker plate for each sample that underwent agitation. Block 3 samples were agitated for 24 hour at room temperature and then analyzed.

C. pH Measurements

The pH each sample was measured using a micro-pH probe. Before the start of analysis the pH probe was calibrated with three pH standards ordered from Fisher. The pH values of the stability samples were measured by transferring 100 μl of each stability sample to a 100 [symbol] L PCR tube. The micro-pH probe was then submerged into the sample and after the value stabilized it was recorded.

D. Conductivity Measurements

The conductivity each sample was measured using a micro-conductivity probe. Before the start of analysis the conductivity probe was calibrated using a Mettler Toledo conductivity standard 1413 μS/cm. The conductivity values of the stability samples were measured by transferring ^{˜}100 μL of each stability sample to a 100 μL PCR tube. The micro-conductivity probe was then submerged into the sample and after the value stabilized it was recorded

E. UV Absorbance Spectroscopy

UV spectroscopy was used to measure the protein concentration in the samples using standard methods (Mach et al., 2011. J Pharm Sci, 100(4), 1214-1227). The mole extinction coefficient at 280 nm for bulk substance was listed as 1.53 mL/mg for commercially obtained samples of TYSABRI. The protein concentrations of tested formulations were measured using a cell path length of 0.0096 cm. Below are the analysis parameters used for this study.

Scan Range: 400 to 200 nm

Average Time (min): 0.1

Date Interval (nm): 1

Scan Rate (nm/min): 600

Cycle Count: 5

F. Size Exclusion Chromatography (SEC) Method

Size Exclusion Chromatography (SEC) was performed using standard methods (Fekete et al., 2014) Trac-Trends in Analytical Chemistry, 63, 76-84) with the following parameters:

All the analysis was conducted using a Thermo Scientific Ultimate 3000 (Thermo Fisher Scientific Waltham, Mass.) operated at room temperature. The flow rate for the separation was 0.1 mL/min and detector was set to 235 nm and 280 nm. After the flow rate and the UV lamp were stable, the samples was injected onto Waters Acquity BEH-200 SEC, 1.7 μm 4.6×300 mm SEC column which was set to 25 C°. The sample was separated under isocratic conditions using the mobile phase 100 mM Sodium Phosphate, 150 mM Sodium Sulfate, 5% 1-Propanol 0.02% Sodium Azide pH=6.3. During the analysis the samples were stored at 8° C.

Method Parameters

Column: Waters Acquity BEH-200 SEC, 1.7 μm 4.6×300 mm

Analysis Buffer: 100 mM Sodium Phosphate, 150 mM Sodium Sulfate, 5% 1-Propanol

0.02% Sodium Azide pH=6.3

Flow rate: 0.1 mL/min

Column temperature: 25° C.

Detection: 235 nm and 280 nm

Injection volume: 0.2 μL

Sample temperature: 8° C.

G. RP HPLC Method

Reverse Phase HPLC was employed using standard methods (Kastner, M. (1999). Protein liquid chromatography. Amsterdam; New York: Elsevier) to analyze stability samples. Below is a summary of the parameters used for the analysis.

All the analysis was conducted using a Thermo Scientific Ultimate 3000 (Thermo Fisher Scientific Waltham, Mass.) operated at room temperature. The flow rate for the separation was 0.5 mL/min and detector was set to 214 nm. After the flow rate and the UV lamp were stable, the samples was injected onto Sepax Proteomix RP-1000, 5 μm 4.6×100 mm which was set to 70 C°. The mobile phases used for the separation of the sample were mobile phase A (0.1% TFA (v/v) in ultra-pure water) and mobile phase B (0.1% TFA (v/v) 50% 1-Propanol, 50% ACN in water) The sample was separated using a gradient were the separation of the protein sample occurred when mobile phase B was increased from 25% to 40% over 35 minute time period. The next part of separation method the percent mobile phase B was increased to 95% to remove any hydrophobic material remaining on the column. The last part of the separation the column was equilibrated to 25% B for 10 minutes. During the analysis the samples were stored at 8° C.

Method Parameters

Column: Sepax Proteomix RP-1000, 5 μm 4.6×100 mm

Mobile Phase A: 0.1% TFA (v/v) in ultra-pure water

Mobile Phase B: 0.1% TFA (v/v) 50% 1-Propanol, 50% ACN in water

Flow rate: 0.5 mL/min

Column temperature: 70° C.

Detection: 214 nm

Injection volume: 1 μL

Sample temperature: Approx. 5° C.

Run time: 55 minutes

H. HIC Method

Hydrophobic Interaction Chromatography (HIC) was employed using standard methods (Kastner, M. (1999). Protein liquid chromatography. Amsterdam; New York: Elsevier) to analyze stability samples. Below is a summary of the parameters used for the analysis.

All the analysis was conducted using a Thermo Scientific Ultimate 3000 (Thermo Fisher Scientific Waltham, Mass.) operated at room temperature. The flow rate for the separation was 1 mL/min and detector was set to 280 nm. After the flow rate and the UV lamp were stable, the samples was injected onto Proteomix HIC Butyl-NP5 4.6×100 mm, 5 μm which was set to 5 C°. The mobile phases used for the separation of the sample were mobile phase A (1.8 M Ammonium Sulfate, 0.05M TRIS, pH 9) and mobile phase B (0.025M TRIS, pH 9) The sample was separated using a gradient were the separation of the protein sample occurred when mobile phase B was increased from 0% to 100% over 12 minute time period. The last part of the separation the column was equilibrated to 0% B for 3 minutes. During the analysis the samples were stored at 8° C.

Method Parameters

Column: Proteomix HIC Butyl-NP5 4.6×100 mm, 5 μm

Mobile Phase A: 1.8 M Ammonium Sulfate, 0.05M TRIS, pH 9

Mobile Phase B: 0.025M TRIS, pH 9

Flow rate: 1 mL/min

Column temperature: 5 C.°

Detection: 220 nm

Injection volume: 2μL

Sample temperature: 5 C.°

I. CEX Method

Cation Exchange Chromatograph (CEX) was employed using standard methods (Fekete et al., 2015. Journal of Pharmaceutical and Biomedical Analysis, 102, 282-289) to analyze stability samples. Below is a summary of the parameters used for the analysis.

All the analysis was conducted using a Thermo Scientific Ultimate 3000 (Thermo Fisher Scientific Waltham, Mass.) operated at room temperature. The flow rate for the separation was 1 mL/min and detector was set to 280 nm. After the flow rate and the UV lamp were stable, the samples was injected onto SEPAX Proteomic SCX-NP5 4.6×250 mm 5 μm which was set to 25 C°. The mobile phases used for the separation of the sample were mobile phase A (2.4 mM Tris/1.5 mM Imidazole/11.6 mM Piperazine, pH 6), mobile phase B (2.4 mM Tris/1.5 mM Imidazole/11.6 mM Piperazine, pH 9.5) and mobile phase C (2.4 mM Tris/1.5 mM Imidazole/11.6 mM Piperazine/0.2M NaCl pH 9.5). The sample was separated using a gradient were the separation of the protein sample occurred when mobile phase B was increased from 0% to 75% over 48 minute time period. The next part of separation method the percent mobile phase C was increased to 100% to clean the column. The last part of the separation the column was equilibrated to 0% B for 25 minutes. During the analysis the samples were stored at 8° C.

Method Parameters

Column Information: SEPAX Proteomic SCX-NP5 4.6×250 mm 5 μm

Mobile Phase A: 2.4 mM Tris/1.5 mM Imidazole/11.6 mM Piperazine, pH 6

Mobile Phase B: 2.4 mM Tris/1.5 mM Imidazole/11.6 mM Piperazine, pH 9.5

Mobile Phase C: 2.4 mM Tris/1.5 mM Imidazole/11.6 mM Piperazine/0.2M NaCl pH 9.5

Flow rate: 1 mL/min

Column temperature: 25° C.

Detection: 280 nm

Injection volume: 10 μL

Sample temperature: Approx. 8° C.

Run time: 80 minutes

J. CE-SDS Analysis

Analysis by CE-SDS was conducted under reducing conditions utilizing a method adapted from the SOP published by Beckman-Coulter for determining IgG purity/heterogeneity (Ganzler et al., 1992 Anal. Chem. 64, 2665-2671). Briefly, the antibody was diluted with DDI water to 6 mg/mL, denatured by adding sample buffer (0.1 M Tris/1.0% SDS, pH 8.0), and reduced via addition of 2-mercaptoethanol; the final antibody concentration was 1.2 mg/mL. Denaturing and reduction was facilitated by heating the sample at 70° C. for 10 min. The sample was cooled for 10 min at room temperature prior to analysis. A centrifuge step (300 g, 5 min) was employed prior to heating the sample and directly after the cooling it. CE analysis was conducted using a Beckman Coulter P/ACE MDQ system operated at ambient temperature with a 30 cm total length (20 cm effective, 50 [symbol] m i.d.) capillary. Prior to sample introduction, the capillary was sequentially rinsed with 0.1 M NaOH, 0.1M HCL, DDI water, and SDS-gel buffer solution. Sample was injected electrokinetically at 5 kV for 30s followed by separation at 30 kV for 30 min. For both injection and separation, the instrument was operated in reverse polarity mode. Antibody fragments were detected using absorbance at 214 nm (4 Hz acquisition) and time-normalized areas reported for measured peaks.

K. Flow Imaging Analysis

The sub-visible particles were measured using a micro flow imaging system (Protein Simple MFI 5200). The samples were first degassed at 75 torr for 20 minutes at ambient condition. The flow cell was then purged with 200 μl of these samples. Additional 70-100 μ of samples were used to optimize the illumination. Data was collected from 150-230 μl of samples and analyzed using MVAS 1.4 software.

### Example 1—Preparation of Natalizumab

This example describes the preparation of natalizumab for use in the stability studies described herein.

Natalizumab may be prepared either de novo or through purification/isolation from a commercially available source. The steps below describe production of natalizumab by culturing cells transformed or transfected with a vector containing natalizumab nucleic acid. Alternative methods, which are well known in the art, may also be employed to prepare natalizumab. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of natalizumab may be chemically synthesized separately and combined using chemical or enzymatic methods to produce natalizumab.

### A. Recombinant Production

DNA encoding the heavy and light chains of natalizumab may be inserted into an expression vector appropriate for the host cell to express natalizumab.

Host cells transfected or transformed with expression or cloning vectors described herein for natalizumab production may be cultured in conventional nutrient media modified as appropriate for inducing promoters, selecting transformants, and/or amplifying the genes encoding the desired sequences. The culture conditions, such as media, temperature, pH and the like, can be selected by the skilled artisan using well-known approaches without undue experimentation. For example, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation, which means introduction of DNA into the host so that the DNA is replicable, either as an extrachromosomal or by chromosomal integrant, are known to the ordinarily skilled artisan, for example, CaCl₂, CaPO₄, liposome-mediated, polyethylene-glycol/DMSO and electroporation.

Natalizumab is expressed in a recombinant NS/0 (murine myeloma) cell line. NS/0 cells derived from a single vial of the Working Cell Bank are grown in increasing volumes of shaker flasks and bioreactors, to obtain an inoculum for the production bioreactor (volume of 15,000 litre). The contents of the production bioreactor are harvested and the conditioned medium is obtained using membrane filtration.

Natalizumab may be recovered from culture medium or from host cell lysates. If membrane-bound, it can be released from the membrane using a suitable detergent solution (e.g Triton-X 100) or by enzymatic cleavage. Cells employed in expression of natalizumab can be disrupted by various physical or chemical means, such as freeze-thaw cycling, sonication, mechanical disruption, or cell lysing agents.

It may be desired to purify natalizumab from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SD S-PAGE; ammonium sulfate precipitation; gel filtration using, for example, Sephadex G-75; protein A Sepharose columns to remove contaminants such as IgG. Various methods of protein purification may be employed and such methods are known in the art and described for example in Deutscher, Methods in Enzymology, 182 (1990); Scopes, Protein Purification: Principles and Practice, Springer-Verlag, New York (1982).

The natalizumab composition prepared from the cells can be purified using, for example, hydroxylapatite chromatography, gel electrophoresis, dialysis, and affinity chromatography, with affinity chromatography being the preferred purification technique. Protein A can be used to purify antibodies that are based on human γ1, γ2 or γ4 heavy chains (Lindmark et al., J. Immunol. Meth. 62:1-13 (1983)). Natalizumab has γ4 heavy chains. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available for purification of natalizumab.

Following any preliminary purification step(s), the mixture comprising natalizumab and contaminants may be subjected to low pH hydrophobic interaction chromatography using an elution buffer at a pH between about 2.5-4.5, preferably performed at low salt concentrations (e.g., from about 0-0.25M salt).

B. Purifying Natalizumab from a Commercially-Available Formulation

Alternatively, natalizumab may be purified from a commercially available preparation such as Tysabri®. Natalizumab can be purified away from other formulation components in Tysabri® using, for example, hydroxyapatite chromatography, gel electrophoresis, dialysis, affinity chromatography, and/or any other applicable purification techniques, including but not limited to Protein A chromatography, fractionation on an ion-exchange column, ethanol precipitation, reverse phase HPLC, chromatography on silica, chromatography on heparin SEPHAROSET®, an anion or cation exchange resin chromatography (such as a polyaspartic acid column), chromatofocusing, SDS-PAGE, and ammonium sulfate precipitation.

For dialysis purification, 100 μL of Tysabri® is placed into Mini Dialysis units with a 3.5 MWCO and dialyzed in 1 L of sodium phosphate buffer for 24 hours at 4 to 8° C. A few samples did experience a small increase in volume due to the dialysis, but never to extent that the concentration of the polysorbate 80 dropped below the CMC (critical micelle concentration).

Material used for experiments in Study Blocks 1 and 2 (described below) was dialyzed using 10,000 MWCO Slide-A-Lyzers in different formulation buffers for 24 hours at a temperature range between 4 to 8° C. After dialysis the protein concentration was measured by UV and sample pH was measured. The concentration of samples was 20±1 mg/mL, which was adjusted if the sample concentration fell out of above range. For samples that lost volume during dialysis, additional formulation buffer was added to reach the target concentration. Once the targeted protein concentration (see method below) and pH of the samples were within experimental parameters, the samples were filtered through 0.22 μM sterile filters into sterile vials in a biosafety hood. The samples were then placed on stability depending on the study design for the block of experiments.

A process designed to minimize the amount of material lost during the buffer exchange step was used for experiments in Study Blocks 3 and 4 (also described below). Here, surfactant-free Tysabri was deformulated using Utralcel with a 30,000 MWCO vs different formulations buffers for 24 hours. The loaded UtraCel were placed on a shaker plate set to 180 rpm with at 4 degrees C. for 24 hours with three buffer outs. Once the targeted protein concentration (20 mg/mL) and pH of the samples were within experimental parameters, the samples were similarly filtered through 0.22 μM sterile filters into sterile vials in a biosafety hood. The samples were then placed on stability depending on the study design for the block of experiments.

The protein concentration for each formulation may be measured by UV absorbance spectroscopy, using a calculated experimental molar absorptivity based on reported concentration of Tysabri®, 50 mg/mL. The protein concentration may be adjusted using, e.g., a spin concentrator, where the sample is placed in the spin concentrator and rotated at 14,000 RPM for 30 to 60 secs. The protein concentration is then checked with UV. After the targeted protein concentration around 50 mg/mL is reached the samples are filtered through 0.22 μM sterile filters as described above.

Natalizumab prepared as described above was used in formulation studies described in the Examples below, according to the following outline:

Outline of Study Blocks 1-4

The goal of the Study 1-3 experimental blocks was to assess the effects of pH, buffer, amino acids and polyols on the chemical and physical stability of natalizumab. Study 1 assessed a range of buffers to assess stability at pH values ranging between 4 and 8. Study 2 assessed a range of excipients, including sugars, polyols and amino acids, at pH values ranging between 5 and 6.5. Study 3 assessed a range of surfactants. Based on analyses of data from these three study blocks, study block 4 was designed to more comprehensively assess specific formulations.

The design and results of Studies 1-4 are presented in the Tables below.

### Index of Tables

### Table 1. Study Design for Block 1 Studies

Table 2. pH, Conductivity, UV and Turbidity measurement for Block 1 at T=0

### Table 3. Visual Inspection for Color and Particles Block 1 at T=0

Table 4. Reverse Phase data for Block 1 study at T=0
Table 5. Size Exclusion Chromatography data for Block 1 study at T=0
Table 6. HIC data for Block 1 study at T=0
Table 7. CEX data for Block 1 study at T=0
Table 8. CE-SDS data for Block 1 at T=0
Table 9. pH, Conductivity, UV and Turbidity measurement for Block 1 at 2 weeks at 40 C°
Table 10. Visual Inspection for Color and Particles for Block 1 at 2 weeks at 40 C°
Table 11. Reverse Phase data for the Block 1 at 2 weeks at 40 C°
Table 12. SEC data for the Block 1 at 2 weeks at 40 C°
Table 13. HIC data for the Block 1 at 2 weeks at 40 C°
Table 14. CEX data for the Block 1 at 2 weeks at 40 C°
Table 15. CE-SDS data for Block 1 at T=2 weeks at 40 C.°
Table 16. pH, Conductivity, UV and Turbidity measurement for Block 1 at 4 weeks at 25 C°
Table 17. Visual Inspection for Color and Particles for Block 1 at 4 weeks at 25 C°
Table 18. Reverse Phase data for the Block 1 at 4 weeks at 25 C°
Table 19. SEC data for the Block 1 at 4 weeks at 25 C°
Table 20. HIC data for the Block 1 at 4 weeks at 25 C°
Table 21. CEX data for the Block 1 at 4 weeks at 25 C°
Table 22. CE-SDS data for Block 1 at T=4 weeks at 25 C.°
Table 23. pH, Conductivity, UV and Turbidity measurement for Block 1 at 13 weeks at 5 C°
Table 24. Visual Inspection for Color and Particles for Block 1 at 13 weeks at 5 C°
Table 25. Reverse Phase data for the Block 1 at 13 weeks at 5 C°
Table 26. SEC data for the Block 1 at 13 weeks at 5 C°
Table 27. HIC data for the Block 1 at 13 weeks at 5 C°
Table 28. CEX data for the Block 1 at 13 weeks at 5 C°
Table 29. CE-SDS data for Block 1 at T=13 weeks at 5 C.°

### Table 30. Study Design for Block 2 Studies

Table 31. pH, Conductivity, UV and Turbidity measurement for Block 2 at T=0

### Table 32. Visual Inspection for Color and Particles for Block 2 at T=0

Table 33. Reverse Phase data for the Block 2 at T=0
Table 34. SEC data for the Block 2 at T=0
Table 35. HIC data for the Block 2 at T=0
Table 36. CEX data for the Block 2 at T=0
Table 37. Ce-SDS data for the Block 2 at T=0
Table 38. Flow imaging data for Total Particle Count in Block 2 samples at T=0
Table 40. pH, Conductivity, UV and Turbidity measurement for Block 2 at 2 weeks at 40 C°
Table 41. Visual Inspection for Color and Particles for Block 2 at 2 weeks at 40 C°
Table 42. Reverse Phase data for Block 2 at 2 weeks at 40 C°
Table 43. SEC data for Block 2 at 2 weeks at 40 C°
Table 44. HIC data for Block 2 at 2 weeks at 40 C°
Table 45. CEX data for Block 2 at 2 weeks at 40 C°
Table 46. pH, Conductivity, UV and Turbidity measurement for Block 2 at 4 weeks at 25 C°
Table 47. Visual Inspection for Color and Particles for Block 2 at 4 weeks at 25 C°
Table 48. Reverse Phase data for Block 2 at 4 weeks at 25 C°
Table 49. SEC data for Block 2 at 4 weeks at 25 C°
Table 50. HIC data for Block 2 at 4 weeks at 25 C°
Table 51. CEX data for Block 2 at 4 weeks at 25 C°
Table 52. CE-SDS data for Block 2 at 4 weeks at 25 C°
Table 53. pH, Conductivity, UV and Turbidity measurement for Block 2 at 13 weeks at 5 C°
Table 54. Visual Inspection for Color and Particles for Block 2 at 13 weeks at 5 C°
Table 55. Reverse Phase data for Block 2 at 13 weeks at 5 C°
Table 56. SEC data for Block 2 at 13 weeks at 5 C°
Table 57. HIC data for Block 2 at 13 weeks at 5 C°
Table 58. CEX data for Block 2 at 13 weeks at 5 C°
Table 59. CE-SDS data for Block 2 at 13 weeks at 5 C°
Table 60. pH, Conductivity, UV and Turbidity measurement for Block 2 Freeze Thaw Studies after 5 freeze thaws
Table 61. Reverse Phase data for Block 2 Freeze Thaw Studies after 5 freeze thaws
Table 62. SEC data for Block 2 Freeze Thaw Studies after 5 freeze thaws
Table 63. HIC data for Block 2 Freeze Thaw Studies after 5 freeze thaws
Table 64. CEX data for Block 2 Freeze Thaw Studies after 5 freeze thaws
Table 65. CE-SDS data for Block 2 Freeze Thaw Studies after 5 freeze thaws
Table 66. Total Particle Count by flow imaging for Block 2 samples after five F/T cycles and storage at −20° C. for eight weeks

### Table 68. Study Design for the Second Freeze Thaw

Table 69. pH, Conductivity, UV and Turbidity measurement for Block 2 Freeze Thaw Studies after 5 freeze thaws
Table 70. Reverse Phase data for Block 2 Second Freeze Thaw Studies after 5 freeze thaws
Table 71. SEC data for Block 2 Second Freeze Thaw Studies after 5 freeze thaws
Table 72. HIC data for Block 2 Second Freeze Thaw Studies after 5 freeze thaws
Table 73. CEX data for Block 2 Second Freeze Thaw Studies after 5 freeze thaws
Table 74. CE-SDS data for Block 2 Second Freeze Thaw Studies after 5 freeze thaws
Table 75. Total Particle Count by flow imaging for Block 2 samples after three and five FIT cycles

### Table 77. Study Design 3

Table 78. pH, Conductivity, UV and Turbidity measurement for Block 3 at T=0

### Table 79. Visual Inspection for Color and Particles for Block 3 at T=0

Table 80. Reverse Phase data for Block 3 at T=0
Table 81. SEC data for Block 3 at 2 weeks at T=0
Table 82. HIC data for Block 3 at 2 weeks at T=0
Table 83. CEX data for Block 3 at 2 weeks at T=0
Table 84. Total Particle Count by flow imaging for Block 3 samples at T=0
Table 86. UV and Turbidity measurement for Block 3 at 2 weeks, 40 C°
Table 87. Visual Inspection for Color and Particles for Block 3 at 2 weeks, 40 C°
Table 88. Reverse Phase data for Block 3 at 2 weeks, 40 C°
Table 89. SEC data for Block 3 at 2 weeks, 40 C°
Table 90. HIC data for Block 3 at 2 weeks, 40 C°
Table 91. CEX data for Block 3 at 2 weeks, 40 C°
Table 92. UV and Turbidity measurement for Block 3 at 4 weeks, 25 C°
Table 93. Visual Inspection for Color and Particles for Block 3 at 4 weeks, 25 C°
Table 94. Reverse Phase data for Block 3 at 4 weeks, 25 C°
Table 95. SEC data for Block 3 at 2 weeks at 4 weeks, 25 C°
Table 96. HIC data for Block 3 at 2 weeks at 4 weeks, 25 C°
Table 97. CEX data for Block 3 at 2 weeks at 4 weeks, 25 C°
Table 98. Total Particle Count by flow imaging for Block 3 samples after 4 weeks at 25 C°
Table 100. UV and Turbidity measurement for Agitation Study for Block 3

### Table 101. Visual Inspection for Particles for Agitation Study for Block 3

Table 102. Reverse Phase Control data for Agitation Study for Block 3
Table 103. Reverse Phase data for 24 Hours Agitation Study for Block 3
Table 104. Reverse Phase data for 48 Hours Agitation Study for Block 3
Table 105. SEC Control data for Agitation Study for Block 3
Table 106. SEC data for 24 Hours Agitation Study for Block 3
Table 107. SEC data for 48 Hours Agitation Study for Block 3
Table 108. HIC Control data for Agitation Study for Block 3
Table 109. HIC data for 24 Hours Agitation Study for Block 3
Table 110. HIC data for 48 Hours Agitation Study for Block 3
Table 111. CEX Control data for Agitation Study for Block 3
Table 112. CEX data for 24 Hours Agitation Study for Block 3
Table 113. CEX data for 48 Hours Agitation Study for Block 3
Table 114. Total Particle Count by flow imaging for Block 3 samples after agitation

### Table 116. Study Design for the Block 3 Freeze Thaw

Table 117. UV and Turbidity measurement for Block 3 Freeze Thaw Studies after 5 freeze thaws
Table 118. SEC data for Block 3 Second Freeze Thaw Studies after 5 freeze thaws
Table 119. HIC data for Block 3 Second Freeze Thaw Studies after 5 freeze thaws
Table 120. CEX data for Block 3 Second Freeze Thaw Studies after 5 freeze thaws
Table 121. Total Particle Count by flow imaging for Block 3 samples after five F/T cycles

### Table 123. Study Design for the Block 4

Table 124. pH, Conductivity, UV and Turbidity measurement for Block 4 at T=0

### Table 125. Visual Inspection for Color and Particles for Block 4 at T=0

Table 126. Reverse Phase data for Block 4 at T=0
Table 127. SEC data for Block 4 T=0
Table 128. HIC data for Block 4 T=0
Table 129. CEX data for Block 4 T=0
Table 130. MFI data for Block 4 T=0
Table 131. UV and Turbidity measurement for Block 4 for 2 weeks 40 C°
Table 132. Visual Inspection for Color and Particles for Block 4 for 2 weeks 40 C°
Table 133. Reverse Phase data for Block 4 for 2 weeks 40 C°
Table 134. SEC data for Block 4 for 2 weeks 40 C°
Table 135. HIC data for Block 4 for 2 weeks 40 C°
Table 136. CEX data for Block 4 for 2 weeks 40 C°
Table 137. UV and Turbidity measurement for Block 4 for 4 weeks 25 C°
Table 138. Visual Inspection for Color and Particles for Block 4 for 4 weeks 25 C°
Table 139. SEC data for Block 4 for 4 weeks 25 C°
Table 140. CEX data for Block 4 for 4 weeks 25 C°
Table 141. Reverse Phase data for Block 4 for 4 weeks 25 C°
Table 142. HIC data for Block 4 for 4 weeks 25 C°
Table 143. Flow imaging data for Block 4 for 4 weeks 25 C°
Table 144. UV and Turbidity measurement for Block 4 for Three Freeze Thaw −20 C°
Table 145. SEC data for Block 4 for Three Freeze Thaw −20 C.°
Table 146. CEX data for Block 4 for Three Freeze Thaw −20 C.°
Table 147. Reverse Phase data for Block 4 for Three Freeze Thaw −20 C.°
Table 148. HIC data for Block 4 for Three Freeze Thaw −20 C.°
Table 149. UV and Turbidity measurement for Block 4 for Five Freeze Thaw −20 C.°

### Table 150. Visual Inspection for Color and Particles for Block 4 for Five Freeze Thaw −20 C.°

Table 151. SEC data for Block 4 for Five Freeze Thaw −20 C.°
Table 152. CEX data for Block 4 for Five Freeze Thaw −20 C.°
Table 153. UV and Turbidity measurement for Block 4 for Control for −40 C.°

### Table 154. Visual Inspection for Color and Particles for Block 4 for Control for −40 C.°

Table 155. SEC data for Block 4 for Control for −40 C.°
Table 156. UV and Turbidity measurement for Block 4 Three Freeze Thaw for −40 C°

### Table 157. Visual Inspection for Color and Particles for Block 4 Three Freeze Thaw for −40 C°

Table 158. SEC data for Block 4 Block 4 Three Freeze Thaw for −40 C°
Table 159. CEX data for Block 4 Block 4 Three Freeze Thaw for −40 C°

Stability Studies. Stability studies were designed to stress the samples to gain the most information on chemical and physical stability. The samples were stressed at two to three temperatures over the course of 4 to 13 weeks. Samples from all Study Blocks were assessed following two weeks at 40 degrees C. Samples from all Study Blocks were also assessed following four weeks at 25 degrees C. Samples from Study Blocks 1 & 2 were further assessed following thirteen weeks at 5 degrees C.

### Example 2 (Study 1 or Block 1)—Determination of Suitable pH

This example describes experiments to evaluate the stability of natalizumab formulated with various buffers at different pH levels, as shown below in Table 1.

TABLE 1
Buffer Study

| Form | | Acetate | Succinate | His | Phosphate | tris | glycine | NaCl | PS80 |
|---|---|---|---|---|---|---|---|---|---|
| No | pH | (mM) | (mM) | (mM) | (mM) | (mM) | (mM) | (mM) | (%) |
| 1 | 4 | 20 | 0 | 0 | 0 | 0 | 0 | 140 | 0.02 |
| 2 | 4.5 | 20 | 0 | 0 | 0 | 0 | 0 | 140 | 0.02 |
| 3 | 5 | 20 | 0 | 0 | 0 | 0 | 0 | 140 | 0.02 |
| 4 | 5 | 0 | 20 | 0 | 0 | 0 | 0 | 140 | 0.02 |
| 5 | 5 | 0 | 0 | 20 | 0 | 0 | 0 | 140 | 0.02 |
| 6 | 5.5 | 0 | 20 | 0 | 0 | 0 | 0 | 140 | 0.02 |
| 7 | 5.5 | 0 | 0 | 20 | 0 | 0 | 0 | 140 | 0.02 |
| 8 | 6 | 0 | 0 | 0 | 20 | 0 | 0 | 140 | 0.02 |
| 9 | 6.5 | 0 | 0 | 20 | 0 | 0 | 0 | 140 | 0.02 |
| 10 | 6.5 | 0 | 0 | 0 | 20 | 0 | 0 | 140 | 0.02 |
| 11 | 7 | 0 | 0 | 0 | 0 | 20 | 0 | 140 | 0.02 |
| 12 | 7 | 0 | 0 | 0 | 20 | 0 | 0 | 140 | 0.02 |
| 13 | 7.5 | 0 | 0 | 0 | 20 | 0 | 0 | 140 | 0.02 |
| 14 | 7.5 | 0 | 0 | 0 | 0 | 20 | 0 | 140 | 0.02 |
| 15 | 8 | 0 | 0 | 0 | 0 | 0 | 20 | 140 | 0.02 |

| | |
|---|---|
| 16 | Tysabri |

All formulations included 140 mM NaCl and 0.02% polysorbate 80 (PS 80). The tested formulations were compared to the commercial TYSABRI formulation (300 mg natalizumab; 123 mg sodium chloride; 17.0 mg sodium phosphate, monobasic, monohydrate; 7.24 mg sodium phosphate, dibasic, heptahydrate; 3.0 mg polysorbate 80; at pH 6.1).

After two weeks, samples stored at 40° C. were pulled and analyzed by methods listed above, including visual inspection. Samples were also stored for four weeks at 25° C. and 13 weeks at 5° C.

The data, summarized in the tables below, indicate that pH has an impact on stability. For example, there is an abrupt decrease in purity by RP HPLC for a sample stored at 40 C (Table 11). Other than that, the stability is comparable for all of the formulations, except those at pH 7 to 8, where decreases as measured by SEC, HIC, and CEX are observed. The data also indicate that phosphate buffer decreases stability somewhat when making comparison at the same pH value. Therefore, an exemplary formulation, based on Study 1 results, would be between pH 4.5 and 6.5 using a buffer other than phosphate. Interestingly, the stability of a number of the formulations described above exceeded that of the marketed product.

TABLE 2
pH, Conductivity, UV and Turbidity measurement for Block 1 at T = 0

| Form | Time Point | Temp. | | Conductivity | UV | Turbidity | |
|---|---|---|---|---|---|---|---|
| No | (Weeks) | C. ° | pH | (mS/cm) | mg/mL | Slope (400 to 320 nm) | A.I. |
| 1 | 0 | N/A | 4.1 | 16.45 | 20.39 | −2.52E−05 | −0.17 |
| 2 | 0 | N/A | 4.59 | 18.56 | 20.46 | −1.89E−05 | −0.01 |
| 3 | 0 | N/A | 5.07 | 16.76 | 20.85 | −2.30E−05 | 0.22 |
| 4 | 0 | N/A | 5.06 | 18.1 | 20.50 | −1.37E−05 | −0.34 |
| 5 | 0 | N/A | 5.02 | 16.39 | 19.84 | −1.94E−05 | −0.39 |
| 6 | 0 | N/A | 5.54 | 16.04 | 19.79 | −1.46E−05 | 0.11 |
| 7 | 0 | N/A | 5.46 | 15.78 | 20.07 | −1.32E−05 | −0.09 |
| 8 | 0 | N/A | 6.09 | 18.24 | 20.52 | −2.69E−05 | 0.14 |
| 9 | 0 | N/A | 6.45 | 16.05 | 20.65 | −2.68E−05 | 0.48 |
| 10 | 0 | N/A | 6.50 | 17.31 | 20.42 | −2.87E−05 | 0.53 |
| 11 | 0 | N/A | 6.89 | 15.68 | 20.24 | −2.33E−05 | −0.24 |
| 12 | 0 | N/A | 7.09 | 17.54 | 21.18 | −3.10E−05 | 0.36 |
| 13 | 0 | N/A | 7.56 | 17.41 | 20.92 | −2.53E−05 | 0.14 |
| 14 | 0 | N/A | 7.50 | 16.66 | 20.61 | −3.63E−05 | 0.14 |
| 15 | 0 | N/A | 7.94 | 14.62 | 20.17 | −2.10E−05 | −0.11 |
| 16 | 0 | N/A | 6.15 | 15.76 | 20.35 | −1.83E−05 | 0.08 |

TABLE 3
Visual Inspection for Color and Particles Block 1 at T = 0

| Form | Time Point | Temp. | Inspection | Inspection |
|---|---|---|---|---|
| No | (Weeks) | C. ° | Color | Particles |
| 1 | 0 | N/A | Clear | Clear |
| 2 | 0 | N/A | Clear | Clear |
| 3 | 0 | N/A | Clear | Clear |
| 4 | 0 | N/A | Clear | Clear |
| 5 | 0 | N/A | Clear | Clear |
| 6 | 0 | N/A | Clear | Clear |
| 7 | 0 | N/A | Clear | Clear |
| 8 | 0 | N/A | Clear | Clear |
| 9 | 0 | N/A | Clear | Clear |
| 10 | 0 | N/A | Clear | Clear |
| 11 | 0 | N/A | Clear | Clear |
| 12 | 0 | N/A | Clear | Clear |
| 13 | 0 | N/A | Clear | Clear |
| 14 | 0 | N/A | Clear | Clear |
| 15 | 0 | N/A | Clear | Clear |
| 16 | 0 | N/A | Clear | Clear |

TABLE 4
RP-HPLC data for Block 1 study at T = 0

| | | | Reverse Phase | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 1.06 | 98.94 | 0.00 |
| 2 | 0 | N/A | 1.08 | 98.92 | 0.00 |
| 3 | 0 | N/A | 1.13 | 98.87 | 0.00 |
| 4 | 0 | N/A | 1.12 | 98.88 | 0.00 |
| 5 | 0 | N/A | 1.20 | 98.80 | 0.00 |
| 6 | 0 | N/A | 1.12 | 98.88 | 0.00 |
| 7 | 0 | N/A | 1.13 | 98.87 | 0.00 |
| 8 | 0 | N/A | 1.08 | 98.93 | 0.00 |
| 9 | 0 | N/A | 1.04 | 98.96 | 0.00 |
| 10 | 0 | N/A | 1.11 | 98.89 | 0.00 |
| 11 | 0 | N/A | 1.06 | 98.94 | 0.00 |
| 12 | 0 | N/A | 1.13 | 98.87 | 0.00 |
| 13 | 0 | N/A | 1.17 | 98.83 | 0.00 |
| 14 | 0 | N/A | 1.08 | 98.92 | 0.00 |
| 15 | 0 | N/A | 1.11 | 98.89 | 0.00 |
| 16 | 0 | N/A | 1.07 | 98.93 | 0.00 |

TABLE 5
Size Exclusion Chromatography (SEC) data for Block 1 study at T = 0

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 0.72 | 98.61 | 0.67 |
| 2 | 0 | N/A | 0.34 | 98.74 | 0.92 |
| 3 | 0 | N/A | 0.56 | 98.71 | 0.74 |
| 4 | 0 | N/A | 0.72 | 98.60 | 0.68 |
| 5 | 0 | N/A | 0.28 | 98.98 | 0.74 |
| 6 | 0 | N/A | 0.39 | 99.12 | 0.49 |
| 7 | 0 | N/A | 0.26 | 98.67 | 1.08 |
| 8 | 0 | N/A | 0.86 | 98.42 | 0.73 |
| 9 | 0 | N/A | 0.26 | 98.90 | 0.84 |
| 10 | 0 | N/A | 1.59 | 98.00 | 0.41 |
| 11 | 0 | N/A | 0.28 | 99.13 | 0.59 |
| 12 | 0 | N/A | 0.95 | 98.10 | 0.95 |
| 13 | 0 | N/A | 0.67 | 98.57 | 0.76 |
| 14 | 0 | N/A | 0.29 | 98.97 | 0.74 |
| 15 | 0 | N/A | 0.29 | 99.12 | 0.59 |
| 16 | 0 | N/A | 0.29 | 98.94 | 0.78 |

TABLE 6
HIC data for Block 1 study at T = 0

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 7.47 | 92.52 | 0.02 |
| 2 | 0 | N/A | 7.37 | 92.62 | 0.01 |
| 3 | 0 | N/A | 7.46 | 92.52 | 0.02 |
| 4 | 0 | N/A | 7.16 | 92.73 | 0.11 |
| 5 | 0 | N/A | 7.29 | 92.71 | 0.00 |
| 6 | 0 | N/A | 7.54 | 92.43 | 0.04 |
| 7 | 0 | N/A | 7.71 | 92.29 | 0.00 |
| 8 | 0 | N/A | 7.79 | 91.98 | 0.24 |
| 9 | 0 | N/A | 7.83 | 92.18 | 0.00 |
| 10 | 0 | N/A | 7.35 | 92.32 | 0.34 |
| 11 | 0 | N/A | 7.91 | 92.09 | 0.00 |
| 12 | 0 | N/A | 8.15 | 91.70 | 0.16 |
| 13 | 0 | N/A | 8.15 | 91.80 | 0.05 |
| 14 | 0 | N/A | 7.67 | 92.33 | 0.00 |
| 15 | 0 | N/A | 7.60 | 92.41 | 0.00 |
| 16 | 0 | N/A | 7.63 | 92.37 | 0.00 |

TABLE 7
CEX data for Block 1 study at T = 0

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 16.38 | 74.34 | 9.28 |
| 2 | 0 | N/A | 16.50 | 74.20 | 9.30 |
| 3 | 0 | N/A | 16.40 | 74.26 | 9.34 |
| 4 | 0 | N/A | 16.49 | 74.29 | 9.22 |
| 5 | 0 | N/A | 16.52 | 74.09 | 9.39 |
| 6 | 0 | N/A | 16.34 | 74.28 | 9.38 |
| 7 | 0 | N/A | 16.36 | 74.24 | 9.40 |
| 8 | 0 | N/A | 16.39 | 74.21 | 9.40 |
| 9 | 0 | N/A | 16.56 | 73.94 | 9.49 |
| 10 | 0 | N/A | 16.69 | 73.90 | 9.42 |
| 11 | 0 | N/A | 17.04 | 73.55 | 9.42 |
| 12 | 0 | N/A | 16.98 | 73.52 | 9.50 |
| 13 | 0 | N/A | 17.06 | 73.44 | 9.50 |
| 14 | 0 | N/A | 17.34 | 73.09 | 9.57 |
| 15 | 0 | N/A | 16.45 | 74.15 | 9.41 |
| 16 | 0 | N/A | 16.57 | 74.06 | 9.37 |

TABLE 8
CE-SDS data for Block 1 at T = 0

| Form | Time Point | Temp. | Heavy | Light | Other |
|---|---|---|---|---|---|
| No | (Weeks) | C. ° | Chain | Chain | Peaks |
| 1 | 0 | N/A | | | |
| 2 | 0 | N/A | 72.97 | 23.92 | 3.11 |
| 3 | 0 | N/A | 73.00 | 24.23 | 2.78 |
| 4 | 0 | N/A | 73.02 | 25.01 | 1.97 |
| 5 | 0 | N/A | 73.16 | 25.55 | 1.29 |
| 6 | 0 | N/A | 73.56 | 25.18 | 1.26 |
| 7 | 0 | N/A | 72.86 | 25.81 | 1.33 |
| 8 | 0 | N/A | 73.12 | 24.51 | 2.37 |
| 9 | 0 | N/A | 72.65 | 26.06 | 1.29 |
| 10 | 0 | N/A | 72.62 | 26.12 | 1.25 |
| 11 | 0 | N/A | 73.57 | 24.83 | 1.59 |
| 12 | 0 | N/A | 72.26 | 25.99 | 1.74 |
| 13 | 0 | N/A | 73.82 | 24.79 | 1.39 |
| 14 | 0 | N/A | 72.39 | 26.27 | 1.34 |
| 15 | 0 | N/A | 73.67 | 24.80 | 1.54 |
| 16 | 0 | N/A | 73.44 | 25.16 | 1.40 |

TABLE 9
pH, Conductivity, UV and Turbidity measurement for Block 1 at 2 weeks at 40° C.

| Form | Time Point | Temp. | | Conductivity | UV | Turbidity | |
|---|---|---|---|---|---|---|---|
| No | (Weeks) | C. ° | pH | (mS/cm) | mg/mL | Slope (400 to 320 nm) | A.I. |
| 1 | 2 | 40 | N/A | N/A | 20.51 | −4.76E−05 | 1.02 |
| 2 | 2 | 40 | N/A | N/A | 20.10 | −1.79E−05 | 0.91 |
| 3 | 2 | 40 | N/A | N/A | 20.50 | 4.61E−06 | 0.37 |
| 4 | 2 | 40 | N/A | N/A | 20.10 | −6.50E−06 | −0.36 |
| 5 | 2 | 40 | N/A | N/A | 19.94 | 4.23E−06 | 0.10 |
| 6 | 2 | 40 | N/A | N/A | | | |
| 7 | 2 | 40 | N/A | N/A | 20.18 | −2.59E−06 | −0.07 |
| 8 | 2 | 40 | N/A | N/A | 20.25 | −2.26E−05 | −0.25 |
| 9 | 2 | 40 | N/A | N/A | 20.22 | −3.17E−06 | −0.29 |
| 10 | 2 | 40 | N/A | N/A | 20.49 | −1.56E−05 | 0.06 |
| 11 | 2 | 40 | N/A | N/A | 20.20 | −1.29E−05 | −0.55 |
| 12 | 2 | 40 | N/A | N/A | 20.56 | −1.25E−05 | 0.03 |
| 13 | 2 | 40 | N/A | N/A | 20.88 | −1.31E−05 | −0.29 |
| 14 | 2 | 40 | N/A | N/A | 20.22 | −1.76E−05 | −0.45 |
| 15 | 2 | 40 | N/A | N/A | 20.21 | −1.80E−05 | −0.46 |
| 16 | 2 | 40 | N/A | N/A | 20.20 | −7.70E−06 | −0.33 |

TABLE 10
Visual Inspection for Color and Particles
for Block 1 at 2 weeks at 40° C.

| Form | Time Point | Temp. | Inspection | Inspection |
|---|---|---|---|---|
| No | (Weeks) | C. ° | Particles | Color |
| 1 | 2 | 40 | Hazy | Blue |
| 2 | 2 | 40 | Clear | Clear |
| 3 | 2 | 40 | Clear | Clear |
| 4 | 2 | 40 | Clear | Clear |
| 5 | 2 | 40 | Clear | Clear |
| 6 | 2 | 40 | Clear | Clear |
| 7 | 2 | 40 | Clear | Clear |
| 8 | 2 | 40 | Clear | Clear |
| 9 | 2 | 40 | Clear | Clear |
| 10 | 2 | 40 | Clear | Clear |
| 11 | 2 | 40 | Clear | Clear |
| 12 | 2 | 40 | Clear | Clear |
| 13 | 2 | 40 | Clear | Clear |
| 14 | 2 | 40 | Clear | Clear |
| 15 | 2 | 40 | Clear | Clear |
| 16 | 2 | 40 | Clear | Clear |

TABLE 11
Reverse Phase data for the Block 1 at 2 weeks at 40° C.

| | | | Reverse Phase | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 2.92 | 84.60 | 12.49 |
| 2 | 2 | 40 | 1.48 | 98.52 | 0.00 |
| 3 | 2 | 40 | 1.28 | 98.72 | 0.00 |
| 4 | 2 | 40 | 1.28 | 98.72 | 0.00 |
| 5 | 2 | 40 | 1.41 | 98.59 | 0.00 |
| 6 | 2 | 40 | | | |
| 7 | 2 | 40 | 1.23 | 98.77 | 0.00 |
| 8 | 2 | 40 | 1.33 | 98.67 | 0.00 |
| 9 | 2 | 40 | 1.43 | 98.57 | 0.00 |
| 10 | 2 | 40 | 1.58 | 98.42 | 0.00 |
| 11 | 2 | 40 | 1.68 | 98.32 | 0.00 |
| 12 | 2 | 40 | 2.43 | 97.57 | 0.00 |
| 13 | 2 | 40 | 3.39 | 96.61 | 0.00 |
| 14 | 2 | 40 | 2.76 | 97.24 | 0.00 |
| 15 | 2 | 40 | 2.45 | 97.55 | 0.00 |
| 16 | 2 | 40 | 1.39 | 98.61 | 0.00 |

TABLE 12
SEC data for the Block 1 at 2 weeks at 40° C.

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 0.00 | 97.43 | 2.57 |
| 2 | 2 | 40 | 1.22 | 97.96 | 0.82 |
| 3 | 2 | 40 | 0.87 | 98.12 | 1.01 |
| 4 | 2 | 40 | 1.30 | 97.76 | 0.95 |
| 5 | 2 | 40 | 0.64 | 98.32 | 1.05 |
| 6 | 2 | 40 | | | |
| 7 | 2 | 40 | 0.39 | 98.76 | 0.85 |
| 8 | 2 | 40 | 1.32 | 97.94 | 0.74 |
| 9 | 2 | 40 | 0.27 | 98.73 | 1.00 |
| 10 | 2 | 40 | 1.61 | 97.48 | 0.90 |
| 11 | 2 | 40 | 0.35 | 98.71 | 0.94 |
| 12 | 2 | 40 | 1.28 | 97.23 | 1.49 |
| 13 | 2 | 40 | 0.99 | 96.66 | 2.35 |
| 14 | 2 | 40 | 0.53 | 97.46 | 2.01 |
| 15 | 2 | 40 | 1.36 | 96.74 | 1.91 |
| 16 | 2 | 40 | 0.25 | 98.67 | 1.08 |

TABLE 13
HIC data for the Block 1 at 2 weeks at 40° C.

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 12.98 | 87.02 | 0.00 |
| 2 | 2 | 40 | 8.95 | 91.05 | 0.00 |
| 3 | 2 | 40 | 8.80 | 91.20 | 0.00 |
| 4 | 2 | 40 | 8.78 | 91.22 | 0.00 |
| 5 | 2 | 40 | 9.20 | 90.80 | 0.00 |
| 6 | 2 | 40 | | | |
| 7 | 2 | 40 | 9.40 | 90.60 | 0.00 |
| 8 | 2 | 40 | 9.06 | 90.75 | 0.18 |
| 9 | 2 | 40 | 9.52 | 90.48 | 0.00 |
| 10 | 2 | 40 | 9.93 | 89.85 | 0.22 |
| 11 | 2 | 40 | 9.76 | 90.24 | 0.00 |
| 12 | 2 | 40 | 13.53 | 86.43 | 0.04 |
| 13 | 2 | 40 | 17.92 | 82.08 | 0.00 |
| 14 | 2 | 40 | 12.30 | 87.70 | 0.00 |
| 15 | 2 | 40 | 17.97 | 82.03 | 0.00 |
| 16 | 2 | 40 | 9.76 | 90.24 | 0.00 |

TABLE 14
CEX data for the Block 1 at 2 weeks at 40° C.

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 21.35 | 65.53 | 13.11 |
| 2 | 2 | 40 | 10.39 | 76.66 | 12.95 |
| 3 | 2 | 40 | 22.12 | 65.03 | 12.85 |
| 4 | 2 | 40 | 21.17 | 64.59 | 14.46 |
| 5 | 2 | 40 | 21.55 | 64.66 | 13.78 |
| 6 | 2 | 40 | | | |
| 7 | 2 | 40 | 21.66 | 65.52 | 12.82 |
| 8 | 2 | 40 | 23.25 | 65.48 | 11.27 |
| 9 | 2 | 40 | 24.59 | 64.50 | 10.91 |
| 10 | 2 | 40 | 27.73 | 62.33 | 9.94 |
| 11 | 2 | 40 | 27.71 | 61.86 | 10.43 |
| 12 | 2 | 40 | 40.44 | 50.74 | 8.82 |
| 13 | 2 | 40 | 54.38 | 38.36 | 7.26 |
| 14 | 2 | 40 | 34.11 | 56.14 | 9.75 |
| 15 | 2 | 40 | 43.08 | 48.34 | 8.58 |
| 16 | 2 | 40 | 25.10 | 63.67 | 11.22 |

TABLE 15
CE-SDS data for Block 1 at T = 2 weeks at 40 C. °

| Form | Time Point | Temp. | Heavy | Light | Other |
|---|---|---|---|---|---|
| No | (Weeks) | C. ° | Chain | Chain | Peaks |
| 1 | 2 | 40 | 67.80 | 26.60 | 5.60 |
| 2 | 2 | 40 | 74.08 | 22.77 | 3.15 |
| 3 | 2 | 40 | 62.73 | 19.37 | 17.90 |
| 4 | 2 | 40 | 60.50 | 20.59 | 18.91 |
| 5 | 2 | 40 | 72.75 | 26.05 | 1.20 |
| 6 | 2 | 40 | 63.41 | 32.16 | 4.43 |
| 7 | 2 | 40 | 62.40 | 22.36 | 15.24 |
| 8 | 2 | 40 | 72.29 | 26.42 | 1.29 |
| 9 | 2 | 40 | 73.59 | 25.19 | 1.21 |
| 10 | 2 | 40 | 73.90 | 25.04 | 1.05 |
| 11 | 2 | 40 | 73.77 | 25.77 | 0.46 |
| 12 | 2 | 40 | 74.07 | 25.32 | 0.61 |
| 13 | 2 | 40 | 73.21 | 26.22 | 0.58 |
| 14 | 2 | 40 | 73.19 | 25.56 | 1.25 |
| 15 | 2 | 40 | 71.98 | 25.84 | 2.18 |
| 16 | 2 | 40 | 73.75 | 24.75 | 1.50 |

TABLE 16
pH, Conductivity, UV and Turbidity measurement for Block 1 at 4 weeks at 25° C.

| Form | Time Point | Temp. | | Conductivity | UV | Turbidity | |
|---|---|---|---|---|---|---|---|
| No | (Weeks) | C. ° | pH | (mS/cm) | mg/mL | Slope (400 to 320 nm) | A.I. |
| 1 | 4 | 25 | N/A | N/A | 19.92 | −1.8E−05 | 0.07 |
| 2 | 4 | 25 | N/A | N/A | 19.96 | −1.8E−05 | −0.21 |
| 3 | 4 | 25 | N/A | N/A | 20.77 | −1.3E−05 | 1.61 |
| 4 | 4 | 25 | N/A | N/A | 20.20 | −1.7E−05 | −0.07 |
| 5 | 4 | 25 | N/A | N/A | 20.09 | −2.3E−05 | −0.12 |
| 6 | 4 | 25 | N/A | N/A | 20.42 | −4.5E−05 | 0.59 |
| 7 | 4 | 25 | N/A | N/A | 20.19 | −2.5E−05 | 0.14 |
| 8 | 4 | 25 | N/A | N/A | 20.42 | −2.2E−05 | −0.29 |
| 9 | 4 | 25 | N/A | N/A | 20.24 | −1.6E−05 | −0.45 |
| 10 | 4 | 25 | N/A | N/A | 20.33 | 4.8E−06 | 0.19 |
| 11 | 4 | 25 | N/A | N/A | 19.97 | −9.5E−06 | −0.19 |
| 12 | 4 | 25 | N/A | N/A | 20.73 | −2.2E−05 | −0.44 |
| 13 | 4 | 25 | N/A | N/A | 20.68 | −2.0E−05 | 0.27 |
| 14 | 4 | 25 | N/A | N/A | 20.15 | −1.5E−05 | −0.32 |
| 15 | 4 | 25 | N/A | N/A | 20.15 | −1.8E−05 | −0.05 |
| 16 | 4 | 25 | N/A | N/A | 20.07 | −1.7E−05 | −0.43 |

TABLE 17
Visual Inspection for Color and Particles
for Block 1 at 4 weeks at 25° C.

| Form | Time Point | Temp. | Inspection | Inspection |
|---|---|---|---|---|
| No | (Weeks) | C. ° | Color | Particles |
| 1 | 4 | 25 | Clear | Clear |
| 2 | 4 | 25 | Clear | Clear |
| 3 | 4 | 25 | Clear | Clear |
| 4 | 4 | 25 | Clear | Clear |
| 5 | 4 | 25 | Clear | Clear |
| 6 | 4 | 25 | Clear | Clear |
| 7 | 4 | 25 | Clear | Clear |
| 8 | 4 | 25 | Clear | Clear |
| 9 | 4 | 25 | Clear | Clear |
| 10 | 4 | 25 | Clear | Clear |
| 11 | 4 | 25 | Clear | Clear |
| 12 | 4 | 25 | Clear | Clear |
| 13 | 4 | 25 | Clear | Clear |
| 14 | 4 | 25 | Clear | Clear |
| 15 | 4 | 25 | Clear | Clear |
| 16 | 4 | 25 | Clear | Clear |

TABLE 18
Reverse Phase data for the Block 1 at 4 weeks at 25° C.

| | | | Reverse Phase | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 1.14 | 98.79 | 0.07 |
| 2 | 4 | 25 | 1.13 | 98.68 | 0.19 |
| 3 | 4 | 25 | 1.11 | 98.72 | 0.18 |
| 4 | 4 | 25 | 1.12 | 98.74 | 0.14 |
| 5 | 4 | 25 | 1.12 | 98.68 | 0.20 |
| 6 | 4 | 25 | 1.07 | 98.72 | 0.21 |
| 7 | 4 | 25 | 1.14 | 98.70 | 0.16 |
| 8 | 4 | 25 | 1.18 | 98.49 | 0.33 |
| 9 | 4 | 25 | 1.21 | 98.66 | 0.13 |
| 10 | 4 | 25 | 1.15 | 98.61 | 0.23 |
| 11 | 4 | 25 | 1.28 | 98.63 | 0.08 |
| 12 | 4 | 25 | 1.29 | 98.55 | 0.16 |
| 13 | 4 | 25 | 1.31 | 98.53 | 0.15 |
| 14 | 4 | 25 | 1.30 | 98.36 | 0.34 |
| 15 | 4 | 25 | 1.30 | 98.52 | 0.18 |
| 16 | 4 | 25 | 1.16 | 98.56 | 0.28 |

TABLE 19
SEC data for the Block 1 at 4 weeks at 25° C.

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 0.47 | 98.89 | 0.63 |
| 2 | 4 | 25 | 0.40 | 99.06 | 0.54 |
| 3 | 4 | 25 | 0.42 | 99.00 | 0.58 |
| 4 | 4 | 25 | 0.63 | 98.67 | 0.71 |
| 5 | 4 | 25 | 0.32 | 98.96 | 0.73 |
| 6 | 4 | 25 | 0.43 | 98.75 | 0.81 |
| 7 | 4 | 25 | 0.25 | 98.90 | 0.84 |
| 8 | 4 | 25 | 0.95 | 98.22 | 0.83 |
| 9 | 4 | 25 | 0.24 | 99.21 | 0.55 |
| 10 | 4 | 25 | 1.16 | 98.17 | 0.68 |
| 11 | 4 | 25 | 0.27 | 99.27 | 0.46 |
| 12 | 4 | 25 | 0.86 | 98.50 | 0.64 |
| 13 | 4 | 25 | 0.51 | 98.60 | 0.89 |
| 14 | 4 | 25 | 0.34 | 99.09 | 0.57 |
| 15 | 4 | 25 | 0.46 | 98.71 | 0.82 |
| 16 | 4 | 25 | 0.34 | 99.12 | 0.54 |

TABLE 20
HIC data for the Block 1 at 4 weeks at 25° C.

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 8.58 | 91.42 | 0.00 |
| 2 | 4 | 25 | 8.79 | 91.21 | 0.00 |
| 3 | 4 | 25 | 8.40 | 91.60 | 0.00 |
| 4 | 4 | 25 | 8.17 | 91.76 | 0.07 |
| 5 | 4 | 25 | 8.47 | 91.53 | 0.00 |
| 6 | 4 | 25 | 8.72 | 91.28 | 0.00 |
| 7 | 4 | 25 | 8.92 | 91.08 | 0.00 |
| 8 | 4 | 25 | 8.83 | 90.92 | 0.25 |
| 9 | 4 | 25 | 8.98 | 91.02 | 0.00 |
| 10 | 4 | 25 | 8.43 | 91.22 | 0.35 |
| 11 | 4 | 25 | 9.15 | 90.85 | 0.00 |
| 12 | 4 | 25 | 9.58 | 90.29 | 0.14 |
| 13 | 4 | 25 | 10.84 | 89.16 | 0.00 |
| 14 | 4 | 25 | 9.64 | 90.36 | 0.00 |
| 15 | 4 | 25 | 10.47 | 89.53 | 0.00 |
| 16 | 4 | 25 | | | |

TABLE 21
CEX data for the Block 1 at 4 weeks at 25° C.

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 9.27 | 69.19 | 21.55 |
| 2 | 4 | 25 | 21.59 | 64.17 | 14.25 |
| 3 | 4 | 25 | 19.73 | 67.04 | 13.24 |
| 4 | 4 | 25 | 23.09 | 64.03 | 12.88 |
| 5 | 4 | 25 | 22.01 | 63.74 | 14.25 |
| 6 | 4 | 25 | 21.94 | 64.77 | 13.30 |
| 7 | 4 | 25 | 23.80 | 65.09 | 11.11 |
| 8 | 4 | 25 | 24.80 | 63.75 | 11.45 |
| 9 | 4 | 25 | 28.56 | 61.30 | 10.14 |
| 10 | 4 | 25 | 28.64 | 60.56 | 10.79 |
| 11 | 4 | 25 | 41.32 | 49.58 | 9.11 |
| 12 | 4 | 25 | 54.20 | 38.26 | 7.54 |
| 13 | 4 | 25 | 35.22 | 54.64 | 10.14 |
| 14 | 4 | 25 | 42.19 | 49.37 | 8.45 |
| 15 | 4 | 25 | 25.81 | 62.49 | 11.70 |
| 16 | 4 | 25 | 19.49 | 68.68 | 11.83 |

TABLE 22
CE-SDS data for Block 1 at T = 4 weeks at 25° C.

| Form | Time Point | Temp. | Heavy | Light | Other |
|---|---|---|---|---|---|
| No | (Weeks) | C. ° | Chain | Chain | Peaks |
| 1 | 4 | 25 | 75.01 | 24.99 | 0.00 |
| 2 | 4 | 25 | 71.72 | 25.11 | 3.17 |
| 3 | 4 | 25 | 71.58 | 27.08 | 1.34 |
| 4 | 4 | 25 | 72.92 | 27.08 | 0.00 |
| 5 | 4 | 25 | 78.79 | 21.21 | 0.00 |
| 6 | 4 | 25 | 74.58 | 24.90 | 0.52 |
| 7 | 4 | 25 | 73.51 | 25.33 | 1.16 |
| 8 | 4 | 25 | 73.26 | 26.21 | 0.52 |
| 9 | 4 | 25 | 73.69 | 25.27 | 1.04 |
| 10 | 4 | 25 | 72.92 | 26.55 | 0.54 |
| 11 | 4 | 25 | 74.01 | 24.93 | 1.06 |
| 12 | 4 | 25 | 73.88 | 26.12 | 0.00 |
| 13 | 4 | 25 | | | |
| 14 | 4 | 25 | 69.62 | 30.38 | 0.00 |
| 15 | 4 | 25 | 72.10 | 25.45 | 2.45 |
| 16 | 4 | 25 | 70.79 | 29.21 | 0.00 |

TABLE 23
pH, Conductivity, UV and Turbidity measurement for Block 1 at 13 weeks at 5° C.

| Form | Time Point | Temp. | | Conductivity | UV | Turbidity | |
|---|---|---|---|---|---|---|---|
| No | (Weeks) | C. ° | pH | (mS/cm) | mg/mL | Slope (400 to 320 nm) | A.I. |
| 1 | 13 | 5 | 4.08 | 16.27 | 20.19 | −1.78E−05 | 0.01 |
| 2 | 13 | 5 | 4.6 | 16.23 | 20.04 | −2.11E−05 | 0.02 |
| 3 | 13 | 5 | 5.04 | 14.50* | 20.15 | −1.05E−05 | −0.38 |
| 4 | 13 | 5 | 5.03 | 16.37 | 20.20 | −2.51E−05 | 0.65 |
| 5 | 13 | 5 | 4.95 | 15.34 | 20.11 | −2.86E−05 | 0.22 |
| 6 | 13 | 5 | 5.49 | 15.86 | 20.27 | −1.64E−05 | −0.01 |
| 7 | 13 | 5 | 5.43 | 15.12 | 20.06 | −1.58E−05 | −0.36 |
| 8 | 13 | 5 | 6.1 | 16.78 | 20.44 | −2.31E−05 | −0.57 |
| 9 | 13 | 5 | 6.46 | 16.34 | 20.37 | −2.32E−05 | 0.13 |
| 10 | 13 | 5 | 6.51 | 15.94 | 20.28 | −1.60E−05 | −0.37 |
| 11 | 13 | 5 | 6.93 | 12.88 | 20.14 | −2.72E−05 | −0.29 |
| 12 | 13 | 5 | 7.07 | 15.42 | 21.06 | −3.45E−05 | 0.26 |
| 13 | 13 | 5 | 7.52 | 15.35 | 20.89 | −2.90E−05 | −0.08 |
| 14 | 13 | 5 | 7.42 | 15.80 | 20.11 | −3.04E−05 | −0.34 |
| 15 | 13 | 5 | 7.8 | 13.24 | 20.13 | −3.04E−05 | −0.15 |
| 16 | 13 | 5 | 6.17 | 15.35 | 20.54 | −3.58E−05 | 0.28 |

*Note:
Very small amount of volume was available for analysis

TABLE 24
Visual Inspection for Color and Particles
for Block 1 at 13 weeks at 5° C.

| Form | Time Point | Temp. | Inspection | Inspection |
|---|---|---|---|---|
| No | (Weeks) | C. ° | Color | Particles |
| 1 | 13 | 5 | Clear | Clear |
| 2 | 13 | 5 | Clear | Clear |
| 3 | 13 | 5 | Clear | Clear |
| 4 | 13 | 5 | Clear | Clear |
| 5 | 13 | 5 | Clear | Clear |
| 6 | 13 | 5 | Clear | Clear |
| 7 | 13 | 5 | Clear | Clear |
| 8 | 13 | 5 | Clear | Clear |
| 9 | 13 | 5 | Clear | Clear |
| 10 | 13 | 5 | Clear | Clear |
| 11 | 13 | 5 | Clear | Clear |
| 12 | 13 | 5 | Clear | Clear |
| 13 | 13 | 5 | Clear | Clear |
| 14 | 13 | 5 | Clear | Clear |
| 15 | 13 | 5 | Clear | Clear |
| 16 | 13 | 5 | Clear | Clear |

TABLE 25
Reverse Phase data for the Block 1 at 13 weeks at 5° C.

| | | | Reverse Phase | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 13 | 5 | 0.83 | 99.17 | 0.00 |
| 2 | 13 | 5 | 0.84 | 99.16 | 0.00 |
| 3 | 13 | 5 | 0.87 | 99.13 | 0.00 |
| 4 | 13 | 5 | 0.87 | 99.13 | 0.00 |
| 5 | 13 | 5 | 0.88 | 99.12 | 0.00 |
| 6 | 13 | 5 | 0.93 | 99.07 | 0.00 |
| 7 | 13 | 5 | 0.93 | 99.07 | 0.00 |
| 8 | 13 | 5 | 0.90 | 99.10 | 0.00 |
| 9 | 13 | 5 | 0.93 | 99.07 | 0.00 |
| 10 | 13 | 5 | 0.93 | 99.07 | 0.00 |
| 11 | 13 | 5 | 0.90 | 99.10 | 0.00 |
| 12 | 13 | 5 | 0.94 | 99.06 | 0.00 |
| 13 | 13 | 5 | 0.94 | 99.06 | 0.00 |
| 14 | 13 | 5 | 0.95 | 99.05 | 0.00 |
| 15 | 13 | 5 | 0.93 | 99.07 | 0.00 |
| 16 | 13 | 5 | 0.94 | 99.06 | 0.00 |

TABLE 26
SEC data for the Block 1 at 13 weeks at 5° C.

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 13 | 5 | 0.52 | 98.74 | 0.74 |
| 2 | 13 | 5 | 0.39 | 98.87 | 0.74 |
| 3 | 13 | 5 | 0.47 | 98.78 | 0.76 |
| 4 | 13 | 5 | 0.93 | 98.35 | 0.72 |
| 5 | 13 | 5 | 0.29 | 98.93 | 0.78 |
| 6 | 13 | 5 | 0.63 | 98.61 | 0.76 |
| 7 | 13 | 5 | 0.31 | 98.88 | 0.81 |
| 8 | 13 | 5 | 1.35 | 97.88 | 0.78 |
| 9 | 13 | 5 | 0.31 | 98.91 | 0.77 |
| 10 | 13 | 5 | 1.78 | 97.47 | 0.75 |
| 11 | 13 | 5 | 0.37 | 98.84 | 0.78 |
| 12 | 13 | 5 | 1.11 | 98.15 | 0.75 |
| 13 | 13 | 5 | 0.76 | 98.48 | 0.77 |
| 14 | 13 | 5 | 0.38 | 98.84 | 0.77 |
| 15 | 13 | 5 | 0.49 | 98.67 | 0.83 |
| 16 | 13 | 5 | 0.29 | 98.88 | 0.82 |

TABLE 27
HIC data for the Block 1 at 13 weeks at 5° C.

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 13 | 5 | 8.80 | 91.20 | 0.00 |
| 2 | 13 | 5 | 9.04 | 90.96 | 0.00 |
| 3 | 13 | 5 | 8.35 | 91.65 | 0.00 |
| 4 | 13 | 5 | 8.11 | 91.73 | 0.16 |
| 5 | 13 | 5 | 8.41 | 91.59 | 0.00 |
| 6 | 13 | 5 | 8.64 | 91.31 | 0.04 |
| 7 | 13 | 5 | 8.85 | 91.15 | 0.00 |
| 8 | 13 | 5 | 8.48 | 91.22 | 0.30 |
| 9 | 13 | 5 | 8.83 | 91.17 | 0.00 |
| 10 | 13 | 5 | 8.66 | 90.93 | 0.41 |
| 11 | 13 | 5 | 8.63 | 91.37 | 0.00 |
| 12 | 13 | 5 | 8.95 | 90.87 | 0.18 |
| 13 | 13 | 5 | 9.03 | 90.90 | 0.07 |
| 14 | 13 | 5 | 9.10 | 90.90 | 0.00 |
| 15 | 13 | 5 | 9.41 | 90.59 | 0.00 |
| 16 | 13 | 5 | 9.01 | 90.97 | 0.02 |

TABLE 28
CEX data for the Block 1 at 13 weeks at 5° C.

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 13 | 5 | 16.73 | 71.96 | 11.31 |
| 2 | 13 | 5 | 17.37 | 70.74 | 11.89 |
| 3 | 13 | 5 | 17.09 | 71.24 | 11.67 |
| 4 | 13 | 5 | 17.19 | 71.00 | 11.80 |
| 5 | 13 | 5 | 16.88 | 71.14 | 11.98 |
| 6 | 13 | 5 | 16.74 | 71.61 | 11.65 |
| 7 | 13 | 5 | 16.85 | 71.33 | 11.82 |
| 8 | 13 | 5 | 16.37 | 72.42 | 11.21 |
| 9 | 13 | 5 | 16.95 | 71.51 | 11.54 |
| 10 | 13 | 5 | 16.70 | 72.18 | 11.12 |
| 11 | 13 | 5 | 17.66 | 71.01 | 11.33 |
| 12 | 13 | 5 | 17.78 | 70.91 | 11.31 |
| 13 | 13 | 5 | 18.62 | 70.30 | 11.08 |
| 14 | 13 | 5 | 18.47 | 70.41 | 11.12 |
| 15 | 13 | 5 | 20.68 | 68.20 | 11.12 |
| 16 | 13 | 5 | 16.55 | 71.77 | 11.68 |

TABLE 29
CE-SDS data for Block 1 at T = 13 weeks at 5° C.

| Form | Time Point | Temp. | Heavy | Light | Other |
|---|---|---|---|---|---|
| No | (Weeks) | C. ° | Chain | Chain | Peaks |
| 1 | 13 | 5 | 73.50 | 25.14 | 1.37 |
| 2 | 13 | 5 | 73.00 | 25.75 | 1.25 |
| 3 | 13 | 5 | 72.27 | 26.24 | 1.49 |
| 4 | 13 | 5 | 72.39 | 26.13 | 1.48 |
| 5 | 13 | 5 | 73.65 | 24.49 | 1.85 |
| 6 | 13 | 5 | 74.25 | 25.24 | 0.51 |
| 7 | 13 | 5 | 73.18 | 26.11 | 0.71 |
| 8 | 13 | 5 | 73.51 | 25.31 | 1.17 |
| 9 | 13 | 5 | 73.41 | 26.01 | 0.59 |
| 10 | 13 | 5 | 73.42 | 25.25 | 1.34 |
| 11 | 13 | 5 | 72.47 | 25.52 | 2.01 |
| 12 | 13 | 5 | 72.72 | 26.11 | 1.17 |
| 13 | 13 | 5 | 73.66 | 24.94 | 1.41 |
| 14 | 13 | 5 | 72.77 | 26.42 | 0.81 |
| 15 | 13 | 5 | 72.44 | 25.89 | 1.67 |
| 16 | 13 | 5 | 72.82 | 26.01 | 1.17 |

### Example 3 (Study 2 or Block 2)—Natalizumab and a Stabilizer

This example describes experiments to evaluate the effects of various stabilizer types and combinations on properties (e.g., stability) of 15 new natalizumab formulations. The pH ranged from pH 5.5 to 6.5, based on the results of Example 1. A number of potential stabilizers were evaluated, to assess the degree of stabilization as compared with the stabilization provided by 140 mM NaCl (present in the commercial formulation). As in Example 1, the PS 80 level was maintained at 0.02%. All of the samples were stored at the same conditions as used for Study 1. In addition, some of these formulations were agitated as well to assess interfacial stability.

TABLE 30
Study Design for Block 2 Studies

| Form | | Acetate | Citrate | Histidine | Glycine | Arginine | Sorbitol | Trehalose | Mannitol | Sucrose | Proline | NaCl | PS 80 |
|---|---|---|---|---|---|---|---|---|---|---|---|---|---|
| No. | pH | (mM) | (mM) | (mM) | (mM) | (mM) | (mM) | (mM) | (mM) | (mM) | (mM) | (mM) | (%) |
| 1 | 5.5 | 0 | 0 | 20 | 0 | 0 | 0 | 0 | 270 | 0 | 0 | 0 | 0.02 |
| 2 | 5.5 | 0 | 0 | 20 | 0 | 0 | 0 | 0 | 0 | 270 | 0 | 0 | 0.02 |
| 3 | 6.5 | 0 | 0 | 20 | 0 | 0 | 0 | 0 | 270 | 0 | 0 | 0 | 0.02 |
| 4 | 6.5 | 0 | 0 | 20 | 0 | 0 | 0 | 0 | 0 | 190 | 0 | 0 | 0.02 |
| 5 | 5.0 | 0 | 20 | 0 | 0 | 0 | 0 | 0 | 270 | 0 | 0 | 0 | 0.02 |
| 6 | 5.0 | 20 | 0 | 0 | 0 | 0 | 0 | 0 | 270 | 0 | 0 | 0 | 0.02 |
| 7 | 6.5 | 0 | 0 | 20 | 0 | 0 | 0 | 0 | 0 | 0 | 0 | 140 | 0.02 |
| 8 | 5.5 | 20 | 0 | 0 | 270 | 0 | 0 | 0 | 0 | 0 | 0 | 0 | 0.02 |
| 9 | 5.5 | 20 | 0 | 0 | 270 | 0 | 0 | 0 | 0 | 0 | 50 | 0 | 0.02 |
| 10 | 6.0 | 0 | 0 | 20 | 0 | 25 | 0 | 0 | 230 | 0 | 0 | 0 | 0.02 |
| 11 | 6.0 | 0 | 0 | 20 | 0 | 50 | 0 | 0 | 190 | 0 | 0 | 0 | 0.02 |
| 12 | 5.0 | 0 | 0 | 20 | 0 | 0 | 0 | 270 | 0 | 0 | 0 | 0 | 0.02 |
| 13 | 6.0 | 0 | 20 | 0 | 0 | 0 | 270 | 0 | 0 | 0 | 0 | 0 | 0.02 |
| 14 | 5.0 | 20 | 0 | 0 | 230 | 25 | 0 | 0 | 0 | 0 | 0 | 0 | 0.02 |
| 15 | 5.5 | 0 | 0 | 20 | 270 | 0 | 0 | 0 | 0 | 0 | 0 | 0 | 0.02 |

| | |
|---|---|
| 16 | Tysabri |

The results, detailed in the Tables below, indicate that the tested formulations had good stability, with the possible exception of Formulation 4 (slightly less stable by CEX than the other preparations). This suggests that once a suitable pH range is established (e.g., pH 5.0 to 6.5), a wide range of other excipients can provide adequate storage stability. All of the tested formulations appear to be comparable or superior to the commercially-marketed formulation for storage stability at 5° C. over thirteen weeks.

TABLE 31
pH, Conductivity, UV and Turbidity measurement for Block 2 at T = 0

| Form | Time Point | Temp. | | Conductivity | UV | Turbidity | |
|---|---|---|---|---|---|---|---|
| No | (Weeks) | C. ° | pH | (mS/cm) | mg/mL | Slope (400 to 320 nm) | A.I. |
| 1 | 0 | N/A | 5.51 | 1.52 | 20.19 | −2.17E−05 | −0.07 |
| 2 | 0 | N/A | 5.62 | 1.31 | 20.19 | −1.44E−05 | 0.35 |
| 3 | 0 | N/A | 6.47 | 1.04 | 19.96 | −2.13E−05 | 0.24 |
| 4 | 0 | N/A | 6.58 | 0.84 | 21.11 | −2.02E−05 | 0.23 |
| 5 | 0 | N/A | 5.17 | 3.48 | 20.04 | −4.64E−06 | 0.57 |
| 6 | 0 | N/A | 5.00 | 2.83 | 20.12 | −1.84E−05 | −0.04 |
| 7 | 0 | N/A | 6.41 | 13.75 | 20.44 | −2.41E−05 | −0.23 |
| 8 | 0 | N/A | 5.59 | 1.7 | 20.62 | −2.65E−05 | 1.29 |
| 9 | 0 | N/A | 5.56 | 1.78 | 19.97 | −2.57E−05 | 1.08 |
| 10 | 0 | N/A | 5.98 | 3.42 | 19.90 | −1.25E−05 | 0.27 |
| 11 | 0 | N/A | 5.97 | 4.89 | 19.52 | −3.54E−06 | 0.14 |
| 12 | 0 | N/A | 5.07 | 1.52 | 19.17 | −1.71E−05 | −0.09 |
| 13 | 0 | N/A | 6.29 | 3.4 | 20.33 | −1.88E−05 | 0.07 |
| 14 | 0 | N/A | 5.04 | 3.72 | 19.99 | −2.28E−05 | −0.02 |
| 15 | 0 | N/A | 5.51 | 1.6 | 20.37 | −6.63E−06 | −0.27 |
| 16 | 0 | N/A | 6.18 | 14.43 | 21.63 | −9.28E−06 | 0.56 |

TABLE 32
Visual Inspection for Color and Particles for Block 2 at T = 0

| Form | Time Point | Temp. | Inspection | Inspection |
|---|---|---|---|---|
| No | (Weeks) | C. ° | Color | Particles |
| 1 | 0 | N/A | Clear | Clear |
| 2 | 0 | N/A | Clear | Clear |
| 3 | 0 | N/A | Clear | Clear |
| 4 | 0 | N/A | Clear | Clear |
| 5 | 0 | N/A | Clear | Clear |
| 6 | 0 | N/A | Clear | Clear |
| 7 | 0 | N/A | Clear | Clear |
| 8 | 0 | N/A | Clear | Clear |
| 9 | 0 | N/A | Clear | Clear |
| 10 | 0 | N/A | Clear | Clear |
| 11 | 0 | N/A | Clear | Clear |
| 12 | 0 | N/A | Clear | Clear |
| 13 | 0 | N/A | Clear | Clear |
| 14 | 0 | N/A | Clear | Clear |
| 15 | 0 | N/A | Clear | Clear |
| 16 | 0 | N/A | Clear | Clear |

TABLE 33
Reverse Phase data for the Block 2 at T = 0

| | | | Reverse Phase | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 1.10 | 98.90 | 0.00 |
| 2 | 0 | N/A | 1.14 | 98.86 | 0.00 |
| 3 | 0 | N/A | 1.15 | 98.85 | 0.00 |
| 4 | 0 | N/A | 1.15 | 98.85 | 0.00 |
| 5 | 0 | N/A | 1.11 | 98.89 | 0.00 |
| 6 | 0 | N/A | 1.11 | 98.89 | 0.00 |
| 7 | 0 | N/A | 1.14 | 98.86 | 0.00 |
| 8 | 0 | N/A | 1.14 | 98.86 | 0.00 |
| 9 | 0 | N/A | 1.10 | 98.90 | 0.00 |
| 10 | 0 | N/A | 1.16 | 98.84 | 0.00 |
| 11 | 0 | N/A | 1.15 | 98.85 | 0.00 |
| 12 | 0 | N/A | 1.09 | 98.90 | 0.00 |
| 13 | 0 | N/A | 1.09 | 98.91 | 0.00 |
| 14 | 0 | N/A | 1.12 | 98.88 | 0.00 |
| 15 | 0 | N/A | 1.13 | 98.87 | 0.00 |
| 16 | 0 | N/A | 1.14 | 98.86 | 0.00 |

TABLE 34
SEC data for the Block 2 at T = 0

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 0.26 | 98.73 | 1.00 |
| 2 | 0 | N/A | 0.26 | 98.72 | 1.02 |
| 3 | 0 | N/A | 0.29 | 98.69 | 1.02 |
| 4 | 0 | N/A | 0.30 | 98.71 | 1.00 |
| 5 | 0 | N/A | 0.33 | 98.68 | 0.99 |
| 6 | 0 | N/A | 0.34 | 98.66 | 0.99 |
| 7 | 0 | N/A | 0.29 | 98.72 | 1.00 |
| 8 | 0 | N/A | 0.27 | 98.71 | 1.02 |
| 9 | 0 | N/A | 0.28 | 98.71 | 1.01 |
| 10 | 0 | N/A | 0.30 | 98.71 | 0.99 |
| 11 | 0 | N/A | 0.28 | 98.73 | 0.98 |
| 12 | 0 | N/A | 0.33 | 98.67 | 1.00 |
| 13 | 0 | N/A | 0.36 | 98.65 | 0.99 |
| 14 | 0 | N/A | 0.26 | 98.76 | 0.98 |
| 15 | 0 | N/A | 0.23 | 98.77 | 1.00 |
| 16 | 0 | N/A | 0.29 | 98.74 | 0.97 |

TABLE 35
HIC data for the Block 2 at T = 0

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 8.12 | 91.88 | 0.00 |
| 2 | 0 | N/A | 8.02 | 91.98 | 0.00 |
| 3 | 0 | N/A | 8.07 | 91.93 | 0.00 |
| 4 | 0 | N/A | 8.05 | 91.95 | 0.00 |
| 5 | 0 | N/A | 8.05 | 91.95 | 0.00 |
| 6 | 0 | N/A | 8.42 | 91.58 | 0.00 |
| 7 | 0 | N/A | 7.78 | 92.22 | 0.00 |
| 8 | 0 | N/A | 7.98 | 92.02 | 0.00 |
| 9 | 0 | N/A | 8.37 | 91.63 | 0.00 |
| 10 | 0 | N/A | 8.23 | 91.77 | 0.00 |
| 11 | 0 | N/A | 8.25 | 91.75 | 0.00 |
| 12 | 0 | N/A | 8.65 | 91.35 | 0.00 |
| 13 | 0 | N/A | 8.52 | 91.48 | 0.00 |
| 14 | 0 | N/A | 8.66 | 91.34 | 0.00 |
| 15 | 0 | N/A | 8.30 | 91.70 | 0.00 |
| 16 | 0 | N/A | 7.52 | 92.48 | 0.00 |

TABLE 36
CEX data for the Block 2 at T = 0

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 17.93 | 71.28 | 10.79 |
| 2 | 0 | N/A | 17.95 | 71.41 | 10.64 |
| 3 | 0 | N/A | 18.29 | 71.17 | 10.54 |
| 4 | 0 | N/A | 18.10 | 71.36 | 10.55 |
| 5 | 0 | N/A | 17.73 | 71.72 | 10.55 |
| 6 | 0 | N/A | 17.67 | 71.68 | 10.66 |
| 7 | 0 | N/A | 18.08 | 71.32 | 10.60 |
| 8 | 0 | N/A | 18.11 | 71.53 | 10.37 |
| 9 | 0 | N/A | 18.14 | 71.23 | 10.63 |
| 10 | 0 | N/A | 18.20 | 71.28 | 10.51 |
| 11 | 0 | N/A | 18.08 | 71.31 | 10.61 |
| 12 | 0 | N/A | 17.90 | 71.56 | 10.54 |
| 13 | 0 | N/A | 18.09 | 71.34 | 10.57 |
| 14 | 0 | N/A | 18.19 | 71.20 | 10.61 |
| 15 | 0 | N/A | 17.81 | 71.86 | 10.33 |
| 16 | 0 | N/A | 17.88 | 71.82 | 10.30 |

TABLE 37
CE-SDS data for the Block 2 at T = 0

| Form | Time Point | Temp. | Heavy | Light | Other |
|---|---|---|---|---|---|
| No | (Weeks) | C. ° | Chain | Chain | Peaks |
| 1 | 0 | N/A | 72.62 | 25.65 | 1.74 |
| 2 | 0 | N/A | 72.70 | 25.63 | 1.67 |
| 3 | 0 | N/A | 72.67 | 26.29 | 1.04 |
| 4 | 0 | N/A | 70.73 | 27.72 | 1.55 |
| 5 | 0 | N/A | 73.05 | 24.92 | 2.03 |
| 6 | 0 | N/A | 71.79 | 26.33 | 1.88 |
| 7 | 0 | N/A | 73.23 | 25.56 | 1.21 |
| 8 | 0 | N/A | 71.99 | 26.36 | 1.65 |
| 9 | 0 | N/A | 72.21 | 26.66 | 1.13 |
| 10 | 0 | N/A | 72.29 | 26.05 | 1.66 |
| 11 | 0 | N/A | 72.99 | 25.20 | 1.81 |
| 12 | 0 | N/A | 72.65 | 25.55 | 1.80 |
| 13 | 0 | N/A | 72.00 | 27.02 | 0.98 |
| 14 | 0 | N/A | 73.06 | 26.01 | 0.93 |
| 15 | 0 | N/A | 72.03 | 26.70 | 1.27 |
| 16 | 0 | N/A | 72.64 | 25.96 | 1.40 |

TABLE 38
Flow imaging data for Total Particle
Count in Block 2 samples at T = 0

| Form | Time Point | Total Particle Count (counts/mL) | | | | |
|---|---|---|---|---|---|---|
| No | (Hours) | All | 2 uM | 5 uM | 10 uM | 25 uM |
| 1 | 0 | 62920 | 12480 | 1902 | 398 | 63 |
| 2 | 0 | 17596 | 1957 | 193 | 90 | 31 |
| 3 | 0 | 55675 | 11334 | 1491 | 218 | 21 |
| 4 | 0 | 53755 | 12013 | 1696 | 320 | 48 |
| 5 | 0 | 23982 | 5851 | 813 | 149 | 5 |
| 6 | 0 | 39880 | 7519 | 1148 | 527 | 90 |
| 7 | 0 | 45100 | 9779 | 1098 | 304 | 83 |
| 8 | 0 | 45865 | 7746 | 1073 | 401 | 96 |
| 9 | 0 | 37980 | 2862 | 331 | 73 | 10 |
| 10 | 0 | 22049 | 2995 | 347 | 78 | 26 |
| 11 | 0 | 26668 | 4198 | 202 | 50 | 0 |
| 12 | 0 | 83964 | 11769 | 838 | 47 | 0 |
| 13 | 0 | 27923 | 6146 | 288 | 13 | 7 |
| 14 | 0 | 20253 | 3614 | 176 | 55 | 27 |
| 15 | 0 | N/A | N/A | N/A | N/A | N/A |
| 16 | 0 | 102515 | 26280 | 5046 | 1527 | 290 |

TABLE 40
pH, Conductivity, UV and Turbidity measurement
for Block 2 at 2 weeks at 40° C.

| Form | Time Point | Temp. | UV | Turbidity | |
|---|---|---|---|---|---|
| No | (Weeks) | C. ° | mg/mL | Slope (400 to 320 nm) | A.I. |
| 1 | 2 | 40 | 19.82 | 4.38E−06 | −0.70 |
| 2 | 2 | 40 | 19.81 | 7.27E−06 | −0.61 |
| 3 | 2 | 40 | 20.02 | 7.45E−06 | −0.82 |
| 4 | 2 | 40 | 20.89 | −2.37E−05 | 2.79 |
| 5 | 2 | 40 | 19.90 | 1.45E−06 | −0.82 |
| 6 | 2 | 40 | 20.09 | 9.16E−06 | −1.06 |
| 7 | 2 | 40 | 19.19 | 1.13E−06 | −0.36 |
| 8 | 2 | 40 | 20.10 | 8.44E−06 | −0.49 |
| 9 | 2 | 40 | 19.25 | 1.91E−06 | −0.57 |
| 10 | 2 | 40 | 19.70 | −8.93E−06 | −0.64 |
| 11 | 2 | 40 | 19.16 | −4.43E−06 | −1.07 |
| 12 | 2 | 40 | 19.11 | 3.24E−06 | −1.05 |
| 13 | 2 | 40 | 20.06 | −7.35E−07 | −0.98 |
| 14 | 2 | 40 | 20.12 | −1.26E−05 | −0.39 |
| 15 | 2 | 40 | 19.56 | −5.90E−06 | −0.72 |
| 16 | 2 | 40 | 21.44 | −1.43E−05 | −0.56 |

TABLE 41
Visual Inspection for Color and Particles
for Block 2 at 2 weeks at 40° C

| Form | Time Point | Temp. | Inspection | Inspection |
|---|---|---|---|---|
| No | (Weeks) | C. ° | Color | Particles |
| 1 | 13 | 5 | Clear | Clear |
| 2 | 13 | 5 | Clear | Clear |
| 3 | 13 | 5 | Clear | Clear |
| 4 | 13 | 5 | Clear | Clear |
| 5 | 13 | 5 | Clear | Clear |
| 6 | 13 | 5 | Clear | Clear |
| 7 | 13 | 5 | Clear | Clear |
| 8 | 13 | 5 | Clear | Clear |
| 9 | 13 | 5 | Clear | Clear |
| 10 | 13 | 5 | Clear | Clear |
| 11 | 13 | 5 | Clear | Clear |
| 12 | 13 | 5 | Clear | Clear |
| 13 | 13 | 5 | Clear | Clear |
| 14 | 13 | 5 | Clear | Clear |
| 15 | 13 | 5 | Clear | Clear |
| 16 | 13 | 5 | Clear | Clear |

TABLE 42
Reverse Phase data for Block 2 at 2 weeks at 40° C.

| | | | RP | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 1.22 | 98.78 | 0.00 |
| 2 | 2 | 40 | 1.23 | 98.77 | 0.00 |
| 3 | 2 | 40 | 1.40 | 98.60 | 0.00 |
| 4 | 2 | 40 | 1.41 | 98.59 | 0.00 |
| 5 | 2 | 40 | 1.18 | 98.82 | 0.00 |
| 6 | 2 | 40 | 1.19 | 98.81 | 0.00 |
| 7 | 2 | 40 | 1.38 | 98.62 | 0.00 |
| 8 | 2 | 40 | 1.21 | 98.79 | 0.00 |
| 9 | 2 | 40 | 1.25 | 98.75 | 0.00 |
| 10 | 2 | 40 | 1.29 | 98.71 | 0.00 |
| 11 | 2 | 40 | 1.29 | 98.71 | 0.00 |
| 12 | 2 | 40 | 1.17 | 98.83 | 0.00 |
| 13 | 2 | 40 | 1.35 | 98.65 | 0.00 |
| 14 | 2 | 40 | 1.23 | 98.77 | 0.00 |
| 15 | 2 | 40 | 1.25 | 98.75 | 0.00 |
| 16 | 2 | 40 | 1.34 | 98.66 | 0.00 |

TABLE 43
SEC data for Block 2 at 2 weeks at 40° C.

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 0.30 | 98.63 | 1.07 |
| 2 | 2 | 40 | 0.26 | 98.59 | 1.15 |
| 3 | 2 | 40 | 0.34 | 98.42 | 1.23 |
| 4 | 2 | 40 | 0.39 | 98.41 | 1.20 |
| 5 | 2 | 40 | 0.44 | 98.51 | 1.05 |
| 6 | 2 | 40 | 0.55 | 98.37 | 1.08 |
| 7 | 2 | 40 | 0.37 | 98.48 | 1.16 |
| 8 | 2 | 40 | 0.37 | 98.52 | 1.11 |
| 9 | 2 | 40 | 0.36 | 98.54 | 1.10 |
| 10 | 2 | 40 | 0.35 | 98.53 | 1.11 |
| 11 | 2 | 40 | 0.35 | 98.53 | 1.12 |
| 12 | 2 | 40 | 0.40 | 98.49 | 1.11 |
| 13 | 2 | 40 | 0.57 | 98.29 | 1.13 |
| 14 | 2 | 40 | 0.34 | 98.56 | 1.10 |
| 15 | 2 | 40 | 0.24 | 98.64 | 1.12 |
| 16 | 2 | 40 | | | |

TABLE 44
HIC data for Block 2 at 2 weeks at 40° C.

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 10.38 | 89.62 | 0.00 |
| 2 | 2 | 40 | 10.27 | 89.73 | 0.00 |
| 3 | 2 | 40 | 10.47 | 89.53 | 0.00 |
| 4 | 2 | 40 | 10.69 | 89.31 | 0.00 |
| 5 | 2 | 40 | 9.88 | 90.11 | 0.01 |
| 6 | 2 | 40 | 10.31 | 89.67 | 0.02 |
| 7 | 2 | 40 | 9.83 | 90.17 | 0.00 |
| 8 | 2 | 40 | 9.85 | 90.15 | 0.00 |
| 9 | 2 | 40 | 10.60 | 89.40 | 0.00 |
| 10 | 2 | 40 | 10.09 | 89.91 | 0.00 |
| 11 | 2 | 40 | 10.21 | 89.79 | 0.00 |
| 12 | 2 | 40 | 10.63 | 89.34 | 0.01 |
| 13 | 2 | 40 | 11.34 | 88.66 | 0.59 |
| 14 | 2 | 40 | 10.17 | 89.80 | 0.03 |
| 15 | 2 | 40 | 10.45 | 89.55 | 0.53 |
| 16 | 2 | 40 | 9.04 | 90.96 | 0.37 |

TABLE 45
CEX data for Block 2 at 2 weeks at 40° C.

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 22.83 | 65.18 | 11.98 |
| 2 | 2 | 40 | 23.07 | 65.08 | 11.86 |
| 3 | 2 | 40 | 26.54 | 63.25 | 10.22 |
| 4 | 2 | 40 | 27.31 | 62.71 | 9.98 |
| 5 | 2 | 40 | 24.05 | 63.66 | 12.29 |
| 6 | 2 | 40 | 22.70 | 64.78 | 12.52 |
| 7 | 2 | 40 | 25.42 | 64.12 | 10.46 |
| 8 | 2 | 40 | 25.69 | 63.10 | 11.20 |
| 9 | 2 | 40 | 25.36 | 63.36 | 11.28 |
| 10 | 2 | 40 | 24.51 | 64.09 | 11.40 |
| 11 | 2 | 40 | 24.38 | 64.24 | 11.38 |
| 12 | 2 | 40 | 23.14 | 64.28 | 12.58 |
| 13 | 2 | 40 | 26.58 | 63.43 | 9.99 |
| 14 | 2 | 40 | 23.64 | 63.96 | 12.40 |
| 15 | 2 | 40 | 24.13 | 63.79 | 12.08 |
| 16 | 2 | 40 | 25.86 | 63.51 | 10.63 |

TABLE 46
pH, Conductivity, UV and Turbidity measurement
for Block 2 at 4 weeks at 25° C.

| Form | Time Point | Temp. | UV | Turbidity | |
|---|---|---|---|---|---|
| No | (Weeks) | C. ° | mg/mL | Slope (400 to 320 nm) | A.I. |
| 1 | 4 | 25 | 20.38 | −1.33E−05 | −0.01 |
| 2 | 4 | 25 | 20.27 | −4.95E−06 | −0.47 |
| 3 | 4 | 25 | 20.12 | 4.81E−06 | −0.19 |
| 4 | 4 | 25 | 20.37 | −1.16E−05 | −0.46 |
| 5 | 4 | 25 | 20.01 | 9.25E−06 | −0.13 |
| 6 | 4 | 25 | 20.62 | 9.38E−06 | −0.29 |
| 7 | 4 | 25 | 20.60 | −1.11E−05 | 0.30 |
| 8 | 4 | 25 | 20.61 | −7.14E−06 | 0.39 |
| 9 | 4 | 25 | 20.11 | −6.48E−06 | 0.06 |
| 10 | 4 | 25 | 19.76 | −8.10E−06 | −0.41 |
| 11 | 4 | 25 | 19.83 | 2.58E−06 | 0.19 |
| 12 | 4 | 25 | 19.34 | 4.23E−06 | −0.74 |
| 13 | 4 | 25 | 20.67 | 4.82E−06 | 0.09 |
| 14 | 4 | 25 | 20.51 | −1.33E−06 | 0.87 |
| 15 | 4 | 25 | 20.07 | 4.39E−06 | −0.21 |
| 16 | 4 | 25 | 22.05 | 9.61E−06 | 0.17 |

TABLE 47
Visual Inspection for Color and Particles
for Block 2 at 4 weeks at 25° C.

| Form | Time Point | Temp. | Inspection | Inspection |
|---|---|---|---|---|
| No | (Weeks) | C. ° | Color | Particles |
| 1 | 4 | 25 | Clear | Clear |
| 2 | 4 | 25 | Clear | Clear |
| 3 | 4 | 25 | Clear | Clear |
| 4 | 4 | 25 | Clear | Clear |
| 5 | 4 | 25 | Clear | Clear |
| 6 | 4 | 25 | Clear | Clear |
| 7 | 4 | 25 | Clear | Clear |
| 8 | 4 | 25 | Clear | Clear |
| 9 | 4 | 25 | Clear | Clear |
| 10 | 4 | 25 | Clear | Clear |
| 11 | 4 | 25 | Clear | Clear |
| 12 | 4 | 25 | Clear | Clear |
| 13 | 4 | 25 | Clear | Clear |
| 14 | 4 | 25 | Clear | Clear |
| 15 | 4 | 25 | Clear | Clear |
| 16 | 4 | 25 | Clear | Clear |

TABLE 48
Reverse Phase data for Block 2 at 4 weeks at 25° C.

| | | | RP | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 1.10 | 98.90 | 0.00 |
| 2 | 4 | 25 | 1.11 | 98.89 | 0.00 |
| 3 | 4 | 25 | 1.20 | 98.76 | 0.04 |
| 4 | 4 | 25 | 1.16 | 98.83 | 0.01 |
| 5 | 4 | 25 | 1.10 | 98.90 | 0.00 |
| 6 | 4 | 25 | 1.21 | 98.79 | 0.00 |
| 7 | 4 | 25 | 1.17 | 98.83 | 0.00 |
| 8 | 4 | 25 | 1.09 | 98.91 | 0.00 |
| 9 | 4 | 25 | 1.21 | 98.79 | 0.00 |
| 10 | 4 | 25 | 1.17 | 98.83 | 0.00 |
| 11 | 4 | 25 | 1.23 | 98.77 | 0.00 |
| 12 | 4 | 25 | 1.15 | 98.85 | 0.00 |
| 13 | 4 | 25 | 1.15 | 98.85 | 0.00 |
| 14 | 4 | 25 | 1.13 | 98.87 | 0.00 |
| 15 | 4 | 25 | 1.08 | 98.92 | 0.00 |
| 16 | 4 | 25 | 1.16 | 98.84 | 0.00 |

TABLE 49
SEC data for Block 2 at 4 weeks at 25° C.

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 0.46 | 98.74 | 0.80 |
| 2 | 2 | 40 | 0.42 | 98.76 | 0.82 |
| 3 | 2 | 40 | 0.46 | 98.67 | 0.87 |
| 4 | 2 | 40 | 0.92 | 98.22 | 0.86 |
| 5 | 2 | 40 | 0.26 | 98.84 | 0.90 |
| 6 | 2 | 40 | 0.52 | 98.58 | 0.90 |
| 7 | 2 | 40 | 0.27 | 98.87 | 0.86 |
| 8 | 2 | 40 | 1.23 | 97.93 | 0.85 |
| 9 | 2 | 40 | 0.31 | 98.80 | 0.89 |
| 10 | 2 | 40 | 1.57 | 97.55 | 0.88 |
| 11 | 2 | 40 | 0.36 | 98.81 | 0.83 |
| 12 | 2 | 40 | 1.05 | 98.10 | 0.85 |
| 13 | 2 | 40 | 0.77 | 98.33 | 0.90 |
| 14 | 2 | 40 | 0.36 | 98.75 | 0.89 |
| 15 | 2 | 40 | 0.47 | 98.64 | 0.89 |
| 16 | 2 | 40 | 0.27 | 98.89 | 0.84 |

TABLE 50
HIC data for Block 2 at 4 weeks at 25° C.

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 5.86 | 94.14 | 0.00 |
| 2 | 2 | 40 | 6.01 | 93.99 | 0.00 |
| 3 | 2 | 40 | 6.06 | 93.94 | 0.00 |
| 4 | 2 | 40 | 6.20 | 93.80 | 0.00 |
| 5 | 2 | 40 | 6.09 | 93.91 | 0.00 |
| 6 | 2 | 40 | 8.68 | 91.32 | 0.00 |
| 7 | 2 | 40 | 8.85 | 91.15 | 0.00 |
| 8 | 2 | 40 | 8.66 | 91.34 | 0.00 |
| 9 | 2 | 40 | 8.90 | 91.10 | 0.00 |
| 10 | 2 | 40 | 8.47 | 91.53 | 0.00 |
| 11 | 2 | 40 | 8.91 | 91.09 | 0.00 |
| 12 | 2 | 40 | 9.18 | 90.82 | 0.00 |
| 13 | 2 | 40 | 9.28 | 90.72 | 0.00 |
| 14 | 2 | 40 | 9.17 | 90.83 | 0.00 |
| 15 | 2 | 40 | 9.58 | 90.42 | 0.00 |
| 16 | 2 | 40 | 9.36 | 90.64 | 0.00 |

TABLE 51
CEX data for Block 2 at 4 weeks at 25° C.

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 19.58 | 69.99 | 10.43 |
| 2 | 4 | 25 | 19.99 | 69.50 | 10.51 |
| 3 | 4 | 25 | 20.88 | 69.39 | 9.72 |
| 4 | 4 | 25 | 21.77 | 68.58 | 9.66 |
| 5 | 4 | 25 | 20.52 | 68.61 | 10.87 |
| 6 | 4 | 25 | 19.80 | 69.63 | 10.57 |
| 7 | 4 | 25 | 20.93 | 69.42 | 9.65 |
| 8 | 4 | 25 | 20.58 | 69.07 | 10.35 |
| 9 | 4 | 25 | 20.43 | 69.32 | 10.25 |
| 10 | 4 | 25 | 20.15 | 69.76 | 10.08 |
| 11 | 4 | 25 | 20.37 | 69.16 | 10.47 |
| 12 | 4 | 25 | 19.88 | 69.34 | 10.77 |
| 13 | 4 | 25 | 20.62 | 69.44 | 9.93 |
| 14 | 4 | 25 | 19.96 | 69.44 | 10.60 |
| 15 | 4 | 25 | 19.52 | 70.18 | 10.30 |
| 16 | 4 | 25 | 20.14 | 70.04 | 9.82 |

TABLE 52
CE−SDS data for Block 2 at 4 weeks at 25° C.

| Form | Time Point | Temp. | Heavy | Light | Other |
|---|---|---|---|---|---|
| No | (Weeks) | C. ° | Chain | Chain | Peaks |
| 1 | 4 | 25 | 71.41 | 26.05 | 2.55 |
| 2 | 4 | 25 | 72.48 | 26.70 | 0.82 |
| 3 | 4 | 25 | 50.99 | 29.06 | 19.95 |
| 4 | 4 | 25 | 38.21 | 36.34 | 25.44 |
| 5 | 4 | 25 | 73.08 | 2.20 | 24.72 |
| 6 | 4 | 25 | 73.92 | 23.45 | 2.64 |
| 7 | 4 | 25 | 71.05 | 26.11 | 2.84 |
| 8 | 4 | 25 | 59.50 | 40.50 | 0.00 |
| 9 | 4 | 25 | 72.61 | 26.35 | 1.03 |
| 10 | 4 | 25 | 72.49 | 25.68 | 1.83 |
| 11 | 4 | 25 | 69.16 | 3.55 | 27.29 |
| 12 | 4 | 25 | 71.30 | 27.47 | 1.22 |
| 13 | 4 | 25 | 71.73 | 26.33 | 1.94 |
| 14 | 4 | 25 | 71.87 | 27.75 | 0.38 |
| 15 | 4 | 25 | 71.06 | 27.94 | 0.10 |
| 16 | 4 | 25 | 71.70 | 26.05 | 2.25 |

TABLE 53
pH, Conductivity, UV and Turbidity measurement
for Block 2 at 13 weeks at 5° C.

| Form | Time Point | Temp. | UV | Turbidity | |
|---|---|---|---|---|---|
| No | (Weeks) | C. ° | mg/mL | Slope (400 to 320 nm) | A.I. |
| 1 | 13 | 5 | 20.77 | −2.78E−06 | 0.97 |
| 2 | 13 | 5 | 20.33 | 2.04E−05 | −0.25 |
| 3 | 13 | 5 | 20.04 | 1.99E−05 | −0.66 |
| 4 | 13 | 5 | 20.63 | 3.89E−06 | 0.83 |
| 5 | 13 | 5 | 20.68 | −5.14E−06 | 0.98 |
| 6 | 13 | 5 | 20.62 | 1.25E−05 | −0.01 |
| 7 | 13 | 5 | 20.67 | 4.61E−06 | −0.33 |
| 8 | 13 | 5 | 20.86 | 1.56E−06 | 0.44 |
| 9 | 13 | 5 | 20.04 | 2.99E−06 | −0.11 |
| 10 | 13 | 5 | 19.93 | 1.19E−05 | 0.20 |
| 11 | 13 | 5 | 19.84 | 6.65E−06 | −0.13 |
| 12 | 13 | 5 | 19.66 | 1.26E−05 | −0.35 |
| 13 | 13 | 5 | 20.98 | 6.52E−07 | 0.10 |
| 14 | 13 | 5 | 20.18 | 7.89E−06 | −0.19 |
| 15 | 13 | 5 | 20.97 | −3.62E−06 | 1.61 |
| 16 | 13 | 5 | 22.00 | 1.47E−05 | 0.29 |

TABLE 54
Visual Inspection for Color and Particles
for Block 2 at 13 weeks at 5° C.

| Form | Time Point | Temp. | Inspection | Inspection |
|---|---|---|---|---|
| No | (Weeks) | C. ° | Color | Particles |
| 1 | 13 | 5 | Clear | Clear |
| 2 | 13 | 5 | Clear | Clear |
| 3 | 13 | 5 | Clear | Clear |
| 4 | 13 | 5 | Clear | Clear |
| 5 | 13 | 5 | Clear | Clear |
| 6 | 13 | 5 | Clear | Clear |
| 7 | 13 | 5 | Clear | Clear |
| 8 | 13 | 5 | Clear | Clear |
| 9 | 13 | 5 | Clear | Clear |
| 10 | 13 | 5 | Clear | Clear |
| 11 | 13 | 5 | Clear | Clear |
| 12 | 13 | 5 | Clear | Clear |
| 13 | 13 | 5 | Clear | Clear |
| 14 | 13 | 5 | Clear | Clear |
| 15 | 13 | 5 | Clear | Clear |
| 16 | 13 | 5 | Clear | Clear |

TABLE 55
Reverse Phase data for Block 2 at 13 weeks at 5° C.

| | | | RP | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 13 | 5 | 1.12 | 98.88 | 0.00 |
| 2 | 13 | 5 | 1.06 | 98.94 | 0.00 |
| 3 | 13 | 5 | 1.11 | 98.89 | 0.00 |
| 4 | 13 | 5 | 1.14 | 98.86 | 0.00 |
| 5 | 13 | 5 | 1.07 | 98.93 | 0.00 |
| 6 | 13 | 5 | 1.09 | 98.91 | 0.00 |
| 7 | 13 | 5 | 1.05 | 98.95 | 0.00 |
| 8 | 13 | 5 | 1.08 | 98.92 | 0.00 |
| 9 | 13 | 5 | 1.03 | 98.97 | 0.00 |
| 10 | 13 | 5 | 1.00 | 99.00 | 0.00 |
| 11 | 13 | 5 | 1.02 | 98.98 | 0.00 |
| 12 | 13 | 5 | 1.03 | 98.97 | 0.00 |
| 13 | 13 | 5 | 1.02 | 98.98 | 0.00 |
| 14 | 13 | 5 | 1.07 | 98.93 | 0.00 |
| 15 | 13 | 5 | 1.20 | 98.80 | 0.00 |
| 16 | 13 | 5 | 1.17 | 98.83 | 0.00 |

TABLE 56
SEC data for Block 2 at 13 weeks at 5° C.

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 13 | 5 | 18.12 | 72.08 | 9.80 |
| 2 | 13 | 5 | 18.25 | 71.82 | 9.93 |
| 3 | 13 | 5 | 18.95 | 71.57 | 9.48 |
| 4 | 13 | 5 | 18.90 | 71.60 | 9.50 |
| 5 | 13 | 5 | 18.27 | 72.20 | 9.53 |
| 6 | 13 | 5 | 18.48 | 71.45 | 10.07 |
| 7 | 13 | 5 | 18.84 | 71.30 | 9.86 |
| 8 | 13 | 5 | 18.71 | 71.25 | 10.05 |
| 9 | 13 | 5 | 18.52 | 71.65 | 9.83 |
| 10 | 13 | 5 | 18.67 | 71.68 | 9.65 |
| 11 | 13 | 5 | 18.30 | 71.88 | 9.81 |
| 12 | 13 | 5 | 18.31 | 71.80 | 9.88 |
| 13 | 13 | 5 | 18.47 | 71.80 | 9.73 |
| 14 | 13 | 5 | 18.29 | 72.11 | 9.60 |
| 15 | 13 | 5 | 18.14 | 72.29 | 9.57 |
| 16 | 13 | 5 | 18.33 | 72.00 | 9.67 |

TABLE 57
HIC data for Block 2 at 13 weeks at 5° C.

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 13 | 5 | 8.27 | 91.73 | 0.00 |
| 2 | 13 | 5 | 7.87 | 92.13 | 0.00 |
| 3 | 13 | 5 | 8.09 | 91.91 | 0.00 |
| 4 | 13 | 5 | 8.26 | 91.74 | 0.00 |
| 5 | 13 | 5 | 7.89 | 92.11 | 0.00 |
| 6 | 13 | 5 | 8.33 | 91.67 | 0.00 |
| 7 | 13 | 5 | 7.55 | 92.45 | 0.00 |
| 8 | 13 | 5 | 7.51 | 92.49 | 0.00 |
| 9 | 13 | 5 | 8.63 | 91.37 | 0.00 |
| 10 | 13 | 5 | 8.35 | 91.65 | 0.00 |
| 11 | 13 | 5 | 8.28 | 91.72 | 0.00 |
| 12 | 13 | 5 | 8.18 | 91.82 | 0.00 |
| 13 | 13 | 5 | 8.63 | 91.37 | 0.00 |
| 14 | 13 | 5 | 8.24 | 91.76 | 0.00 |
| 15 | 13 | 5 | 8.35 | 91.65 | 0.00 |
| 16 | 13 | 5 | 6.93 | 93.07 | 0.00 |

TABLE 58
CEX data for Block 2 at 13 weeks at 5° C.

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 13 | 5 | 18.12 | 72.08 | 9.80 |
| 2 | 13 | 5 | 18.25 | 71.82 | 9.93 |
| 3 | 13 | 5 | 18.95 | 71.57 | 9.48 |
| 4 | 13 | 5 | 18.90 | 71.60 | 9.50 |
| 5 | 13 | 5 | 18.27 | 72.20 | 9.53 |
| 6 | 13 | 5 | 18.48 | 71.45 | 10.07 |
| 7 | 13 | 5 | 18.84 | 71.30 | 9.86 |
| 8 | 13 | 5 | 18.71 | 71.25 | 10.05 |
| 9 | 13 | 5 | 18.52 | 71.65 | 9.83 |
| 10 | 13 | 5 | 18.67 | 71.68 | 9.65 |
| 11 | 13 | 5 | 18.30 | 71.88 | 9.81 |
| 12 | 13 | 5 | 18.31 | 71.80 | 9.88 |
| 13 | 13 | 5 | 18.47 | 71.80 | 9.73 |
| 14 | 13 | 5 | 18.29 | 72.11 | 9.60 |
| 15 | 13 | 5 | 18.14 | 72.29 | 9.57 |
| 16 | 13 | 5 | 18.33 | 72.00 | 9.67 |

TABLE 59
CE-SDS data for Block 2 at 13 weeks at 5° C.

| Form | Time Point | Temp. | Heavy | Light | Other |
|---|---|---|---|---|---|
| No | (Weeks) | C. ° | Chain | Chain | Peaks |
| 1 | 13 | 25 | 70.81 | 25.42 | 3.77 |
| 2 | 13 | 25 | 71.33 | 27.88 | 0.79 |
| 3 | 13 | 25 | 71.63 | 26.53 | 1.83 |
| 4 | 13 | 25 | 71.12 | 26.75 | 2.13 |
| 5 | 13 | 25 | 72.10 | 25.98 | 1.93 |
| 6 | 13 | 25 | 70.22 | 25.79 | 3.99 |
| 7 | 13 | 25 | 70.17 | 26.03 | 3.80 |
| 8 | 13 | 25 | | | |
| 9 | 13 | 25 | 72.82 | 25.61 | 1.57 |
| 10 | 13 | 25 | 71.90 | 27.27 | 0.83 |
| 11 | 13 | 25 | 71.88 | 25.91 | 2.21 |
| 12 | 13 | 25 | 69.94 | 28.84 | 1.22 |
| 13 | 13 | 25 | 72.69 | 25.45 | 1.86 |
| 14 | 13 | 25 | 71.56 | 26.52 | 1.92 |
| 15 | 13 | 25 | | | |
| 16 | 13 | 25 | 72.35 | 25.85 | 1.80 |

### Freeze Thaw Studies

Some of these formulations from Study 2 were subjected to repeated freezing and thawing. After three and five freeze-thaw (F/T) cycles, the stability was evaluated again. In addition to CE-SDS and the various chromatographic methods, levels of subvisible particles (SVPs) were determined using flow imaging. The particle levels at t0 can be found in Table 38. Some of the Block 2 samples were stored at −20° C. for eight weeks as well after undergoing five FIT cycles.

TABLE 60
pH, Conductivity, UV and Turbidity measurement
for Block 2 samples after five F/T cycles and
storage at −20° C. for eight weeks

| Form | Time Point | Temp. | UV | Turbidity | |
|---|---|---|---|---|---|
| No | (Weeks) | C. ° | mg/mL | Slope (400 to 320 nm) | A.I. |
| 1 | 8 | −20 | 20.28 | −2.12E−05 | 0.30 |
| 2 | 8 | −20 | 20.44 | −1.98E−05 | 0.39 |
| 3 | 8 | −20 | 20.84 | −1.62E−05 | 0.47 |
| 8 | 8 | −20 | 20.95 | −3.20E−05 | 1.74 |
| 11 | 8 | −20 | 20.41 | −3.50E−05 | 1.58 |
| 16 | 8 | −20 | 22.30 | −3.99E−05 | 1.35 |

TABLE 61
Reverse Phase data for Block 2 samples after five F/T cycles
and storage at −20° C. for eight weeks

| | | | RP | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 8 | −20 | 1.31 | 98.69 | 0.00 |
| 2 | 8 | −20 | 1.07 | 98.93 | 0.00 |
| 3 | 8 | −20 | 1.27 | 98.73 | 0.00 |
| 8 | 8 | −20 | 1.11 | 98.89 | 0.00 |
| 11 | 8 | −20 | 1.06 | 98.94 | 0.00 |
| 16 | 8 | −20 | | | |

TABLE 62
SEC data for Block 2 samples after five F/T cycles
and storage at −20° C. for eight weeks

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 8 | −20 | 12.10 | 86.96 | 0.94 |
| 2 | 8 | −20 | 0.27 | 98.85 | 0.88 |
| 3 | 8 | −20 | 9.65 | 89.37 | 0.98 |
| 8 | 8 | −20 | 8.69 | 90.47 | 0.85 |
| 11 | 8 | −20 | 0.63 | 98.43 | 0.94 |
| 16 | 8 | −20 | 0.32 | 98.69 | 0.99 |

TABLE 63
HIC data for Block 2 samples after five F/T cycles
and storage at −20° C. for eight weeks

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 8 | −20 | 7.55 | 80.60 | 11.85 |
| 2 | 8 | −20 | 7.71 | 92.29 | 0.00 |
| 3 | 8 | −20 | 7.39 | 82.56 | 10.05 |
| 8 | 8 | −20 | 7.29 | 84.10 | 8.61 |
| 11 | 8 | −20 | 8.19 | 91.81 | 4.04 |
| 16 | 8 | −20 | 6.67 | 93.33 | 0.00 |

TABLE 64
CEX data for Block 2 samples after five F/T cycles
and storage at −20° C. for eight weeks

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 8 | −20 | 17.37 | 71.26 | 11.37 |
| 2 | 8 | −20 | 18.04 | 72.15 | 9.81 |
| 3 | 8 | −20 | 17.55 | 69.13 | 13.32 |
| 8 | 8 | −20 | 17.89 | 70.29 | 11.82 |
| 11 | 8 | −20 | 18.66 | 71.72 | 9.62 |
| 16 | 8 | −20 | | | |

TABLE 65
CE-SDS data for Block 2 samples after five F/T cycles
and storage at −20° C. for eight weeks

| Form | Time Point | Temp. | Heavy | Light | Other |
|---|---|---|---|---|---|
| No | (Weeks) | C. ° | Chain | Chain | Peaks |
| 1 | 8 | −20 | 72.01 | 27.04 | 0.94 |
| 2 | 8 | −20 | 72.47 | 25.79 | 1.74 |
| 3 | 8 | −20 | 72.72 | 25.51 | 1.77 |
| 8 | 8 | −20 | 72.84 | 25.32 | 1.85 |
| 11 | 8 | −20 | 70.84 | 26.47 | 2.69 |
| 16 | 8 | −20 | 72.41 | 25.81 | 1.78 |

TABLE 66
Total Particle Count by flow imaging for Block 2 samples after five
F/T cycles and storage at −20° C. for eight weeks

| Form | Time Point | Temp. | | Total Particle Count (counts/mL) | | | | |
|---|---|---|---|---|---|---|---|---|
| No | (Weeks) | C. ° | pH | All | 2 uM | 5 uM | 10 uM | 25 uM |
| 1 | 8 | −20 | 5.51 | 102573 | 16113 | 854 | 150 | 12 |
| 2 | 8 | −20 | 5.62 | 20737 | 1728 | 95 | 28 | 17 |
| 3 | 8 | −20 | 6.47 | 26005 | 5859 | 448 | 60 | 15 |
| 8 | 8 | −20 | 5.59 | 6635 | 1454 | 253 | 91 | 14 |
| 11 | 8 | −20 | 5.97 | 4249 | 764 | 168 | 35 | 12 |
| 16 | 8 | −20 | 6.18 | 45877 | 7739 | 572 | 195 | 67 |

Differences were noted in storage stability after eight weeks at −20 C. Formulations 2 and 11, along with the control (Formulation 16) all showed significantly better storage stability in the frozen state than the other compositions. The formulations with the poorest stability profiles all contained excipients that could easily crystallize, such as mannitol or glycine, whereas the more stable one had sucrose or a sufficient amount of arginine to inhibit crystallization. The differences were seen in any method capable to detecting changes in physical stability (SEC, HIC), while chemical stability was largely unaffected.

The stabilities of these formulations were also evaluated immediately after three and five F/T cycles (see Tables 68 through 75). As above, formulations containing mannitol or glycine showed significantly lower stability in the frozen state, and these differences could be seen after only three F/T cycles. In contrast, sucrose, NaCl, and/or mannitol, optionally in combination with arginine, were relatively stable under these stress conditions.

TABLE 68
Study Design for the Second Freeze Thaw Study

| Form | | Acetate | Citrate | Histidine | Glycine | Arginine | Sorbitol | Trehalose | Mannitol | Sucrose | Proline | NaCl | PS 80 |
|---|---|---|---|---|---|---|---|---|---|---|---|---|---|
| No. | pH | (mM) | (mM) | (mM) | (mM) | (mM) | (mM) | (mM) | (mM) | (mM) | (mM) | (mM) | (%) |
| 1 | 5.5 | 0 | 0 | 20 | 0 | 0 | 0 | 0 | 270 | 0 | 0 | 0 | 0.02 |
| 2 | 5.5 | 0 | 0 | 20 | 0 | 0 | 0 | 0 | 0 | 270 | 0 | 0 | 0.02 |
| 3 | 6.5 | 0 | 0 | 20 | 0 | 0 | 0 | 0 | 270 | 0 | 0 | 0 | 0.02 |
| 8 | 5.5 | 20 | 0 | 0 | 270 | 0 | 0 | 0 | 0 | 0 | 0 | 0 | 0.02 |
| 11 | 6.0 | 0 | 0 | 20 | 0 | 50 | 0 | 0 | 190 | 0 | 0 | 0 | 0.02 |
| 15 | 5.5 | 0 | 0 | 20 | 270 | 0 | 0 | 0 | 0 | 0 | 0 | 0 | 0.02 |

| | |
|---|---|
| 16 | Tysabri |

TABLE 69
pH, Conductivity, UV and Turbidity measurement for
Block 2 Freeze Thaw Studies after 5 freeze thaws

| Form | Time Point | Temp. | UV | Turbidity | |
|---|---|---|---|---|---|
| No | (Weeks) | C. ° | mg/mL | Slope (400 to 320 nm) | A.I. |
| 1 | 8 | −20 | 19.91 | −1.28E−05 | −0.06 |
| 2 | 8 | −20 | 19.13 | 2.01E−06 | −0.38 |
| 3 | 8 | −20 | 18.98 | −6.78E−06 | −0.69 |
| 8 | 8 | −20 | 19.62 | −1.06E−07 | −0.67 |
| 11 | 8 | −20 | 19.89 | −9.84E−06 | −0.64 |
| 16 | 8 | −20 | 20.54 | −9.78E−06 | −0.45 |

TABLE 70
Reverse Phase data for Block 2 samples after three and five F/T cycles

| | Control | | | 3 Freeze Thaws | | | 5 Freeze Thaws | | |
|---|---|---|---|---|---|---|---|---|---|
| Form | Before | | After | Before | | After | Before | | After |
| No | Monomer | Monomer | Monomer | Monomer | Monomer | Monomer | Monomer | Monomer | Monomer |
| 1 | 1.09 | 98.91 | 0 | 1.3 | 98.7 | 0 | 1.30 | 98.70 | 0.00 |
| 2 | 1.14 | 98.86 | 0 | 1.31 | 98.69 | 0 | 1.31 | 98.69 | 0.00 |
| 3 | 1.19 | 98.81 | 0 | 1.34 | 98.66 | 0 | 1.34 | 98.66 | 0.00 |
| 8 | 1.14 | 98.86 | 0 | 1.23 | 98.77 | 0 | 1.23 | 98.77 | 0.00 |
| 11 | 1.19 | 98.81 | 0 | 1.23 | 98.77 | 0 | 1.23 | 98.77 | 0.00 |
| 15 | 1.16 | 98.84 | 0 | 1.3 | 98.7 | 0 | 1.30 | 98.70 | 0.00 |
| 16 | 1.19 | 98.81 | 0 | 1.24 | 98.76 | 0 | 1.24 | 98.76 | 0.00 |

TABLE 71
SEC data for Block 2 samples after three and five F/T cycles

| | Control | | | 3 Freeze Thaws | | | 5 Freeze Thaws | | |
|---|---|---|---|---|---|---|---|---|---|
| Form | Before | | After | Before | | After | Before | | After |
| No | Monomer | Monomer | Monomer | Monomer | Monomer | Monomer | Monomer | Monomer | Monomer |
| 1 | 0.2 | 98.87 | 0.93 | 2.67 | 96.41 | 0.92 | 5.42 | 93.47 | 1.11 |
| 2 | 0.19 | 98.87 | 0.94 | 0.2 | 98.85 | 0.95 | 0.20 | 98.81 | 0.98 |
| 3 | 0.2 | 98.86 | 0.95 | 3.86 | 95.1 | 1.03 | 2.79 | 48.87 | 48.33 |
| 8 | 0.25 | 98.81 | 0.94 | 2.64 | 96.41 | 0.95 | 6.62 | 92.29 | 1.09 |
| 11 | 0.22 | 98.84 | 0.94 | 0.33 | 98.73 | 0.94 | 0.47 | 98.48 | 1.05 |
| 15 | 0.2 | 98.87 | 0.93 | 3.7 | 95.42 | 0.88 | 8.68 | 90.24 | 1.08 |
| 16 | 0.25 | 98.8 | 0.95 | 0.29 | 98.76 | 0.95 | 1.12 | 97.84 | 1.04 |

Formulation 1, 3 and 8 all show damage after three freeze thaw events. These are the same formulations that showed damage in the first freeze thaw study.

TABLE 72
HIC data for Block 2 samples after three and five F/T cycles

| | Control | | | 5 Freeze Thaws | | | 5 Freeze Thaws | | |
|---|---|---|---|---|---|---|---|---|---|
| Form | Before | | After | Before | | After | Before | | After |
| No | Monomer | Monomer | Monomer | Monomer | Monomer | Monomer | Monomer | Monomer | Monomer |
| 1 | 6.19 | 93.81 | 0 | 6.37 | 90.96 | 2.67 | 6.81 | 88.62 | 4.56 |
| 2 | 6.13 | 93.87 | 0 | 6.54 | 93.46 | 0 | 6.57 | 93.43 | 0.00 |
| 3 | 6.35 | 93.65 | 0 | 6.56 | 89.88 | 3.55 | 6.59 | 88.83 | 4.58 |
| 8 | 6.25 | 93.75 | 0 | 6.69 | 91.25 | 2.07 | 6.19 | 89.38 | 4.43 |
| 11 | 6.38 | 93.62 | 0 | 6.13 | 92.3 | 1.57 | 6.50 | 93.35 | 0.14 |
| 15 | 6.41 | 93.59 | 0 | 6.08 | 92.26 | 1.65 | 6.28 | 88.65 | 5.06 |
| 16 | 6.41 | 93.59 | 0 | 6.11 | 93.89 | 0 | 6.55 | 91.72 | 1.73 |

TABLE 73
CEX data for Block 2 samples after three and five F/T cycles

| | Control | | | 3 Freeze Thaws | | | 5 Freeze Thaws | | |
|---|---|---|---|---|---|---|---|---|---|
| Form | Before | | After | Before | | After | Before | | After |
| No | Monomer | Monomer | Monomer | Monomer | Monomer | Monomer | Monomer | Monomer | Monomer |
| 1 | 17.88 | 73.08 | 9.04 | 17.89 | 73.28 | 8.83 | 17.49 | 72.82 | 9.69 |
| 2 | 17.80 | 73.48 | 8.71 | 18.14 | 73.07 | 8.79 | 17.79 | 73.61 | 8.60 |
| 3 | 18.34 | 73.54 | 8.11 | 18.04 | 73.29 | 8.67 | 17.97 | 73.26 | 8.77 |
| 8 | 17.93 | 73.15 | 8.92 | 17.90 | 73.25 | 8.85 | 17.63 | 72.49 | 9.88 |
| 11 | 17.99 | 73.00 | 9.01 | 17.93 | 73.03 | 9.04 | 17.94 | 73.32 | 8.74 |
| 15 | 17.85 | 73.32 | 8.82 | 17.93 | 73.29 | 8.78 | 17.42 | 72.25 | 10.32 |
| 16 | 17.84 | 73.28 | 8.88 | 17.97 | 72.98 | 9.05 | 17.52 | 73.19 | 9.29 |

TABLE 74
CE-SDS data for Block 2 samples after three and five F/T cycles

| | |
|---|---|
| Control | 3 Freeze Thaws |

| Form | Heavy | Light | Other | Heavy | Light | Other |
|---|---|---|---|---|---|---|
| No | Chain | Chain | Peaks | Chain | Chain | Peaks |
| 1 | 73.32 | 25.19 | 1.49 | 72.51 | 26.38 | 1.10 |
| 2 | 73.05 | 25.20 | 1.75 | 73.14 | 25.20 | 1.67 |
| 3 | | | | 72.58 | 25.42 | 1.10 |
| 8 | 73.01 | 25.06 | 1.92 | 71.96 | 26.77 | 1.27 |
| 11 | 76.17 | 22.79 | 1.04 | | | |
| 15 | 73.03 | 25.22 | 1.75 | | | |
| 16 | 69.69 | 25.88 | 4.43 | | | |

TABLE 75
Total Particle Count by flow imaging for Block 2 samples after three and five F/T cycles

| | MFI (Silicone Oil) | | | | | | | | | | | | | | |
|---|---|---|---|---|---|---|---|---|---|---|---|---|---|---|---|
| | Control (counts/mL) | | | | | 3 Freeze Thaws (counts/mL) | | | | | 5 Freeze Thaws (counts/mL) | | | | |
| Form | | ≥2 | ≥5 | ≥10 | ≥25 | | ≥2 | ≥5 | ≥10 | ≥25 | | ≥2 | ≥5 | ≥10 | ≥25 |
| No | All | uM | uM | uM | uM | All | uM | uM | uM | uM | All | uM | uM | uM | uM |
| 1 | 27692 | 6417 | 1386 | 264 | 0 | 37301 | 5669 | 376 | 83 | 17 | 40737 | 6401 | 657 | 94 | 9 |
| 2 | 15510 | 1724 | 257 | 69 | 3 | 37378 | 6641 | 856 | 199 | 14 | 29940 | 3445 | 380 | 39 | 0 |
| 3 | 24680 | 4300 | 377 | 65 | 7 | 199480 | 50746 | 7050 | 953 | 93 | 36752 | 7498 | 888 | 115 | 5 |
| 8 | 49800 | 10011 | 639 | 78 | 10 | 68929 | 17357 | 2479 | 530 | 80 | 67138 | 15510 | 916 | 60 | 0 |
| 11 | 31393 | 4258 | 103 | 16 | 11 | 35141 | 7675 | 423 | 64 | 13 | 45928 | 7005 | 275 | 47 | 6 |
| 15 | 20838 | 4516 | 184 | 78 | 33 | 31541 | 9466 | 816 | 75 | 24 | 77171 | 16379 | 846 | 98 | 22 |
| 16 | 26034 | 6498 | 311 | 91 | 27 | 37717 | 11456 | 960 | 110 | 21 | 66319 | 17034 | 979 | 67 | 15 |

### Example 4 (Study 3 or Block 3)—Effect of Surfactants on Natalizumab Stability

This example describes experiments to evaluate the effect of surfactants on the stability of twelve new natalizumab formulations. All of the formulations were prepared with surfactant-free material; and different surfactants were then added to evaluate the impact of this class of excipients. Also included was PEG 3350, which can protect against interfacial damage in some systems. The design is shown in Table 77. All samples were at the same pH (6.0) in the same diluents (20 mM His, 140 mM NaCl). The first study was designed to determine if any of the surfactants had an impact on storage stability (Tables 78 through 98).

TABLE 77
Study 3 Design

| Form | | Histidine | NaCl | PS 80 | PS 20 | F-68 | DDM | PEG 3350 |
|---|---|---|---|---|---|---|---|---|
| No. | pH | (mM) | (mM) | (%) | (%) | (%) | (%) | (%) |
| 1 | 6.0 | 20 | 140 | 0 | 0 | 0 | 0 | 0 |
| 2 | 6.0 | 20 | 140 | 0.01 | 0 | 0 | 0 | 0 |
| 3 | 6.0 | 20 | 140 | 0.02 | 0 | 0 | 0 | 0 |
| 4 | 6.0 | 20 | 140 | 0 | 0.01 | 0 | 0 | 0 |
| 5 | 6.0 | 20 | 140 | 0 | 0.02 | 0 | 0 | 0 |
| 6 | 6.0 | 20 | 140 | 0 | 0.05 | 0 | 0 | 0 |
| 7 | 6.0 | 20 | 140 | 0 | 0 | 0.1 | 0 | 0 |
| 8 | 6.0 | 20 | 140 | 0 | 0 | 0.2 | 0 | 0 |
| 9 | 6.0 | 20 | 140 | 0 | 0 | 0 | 0.1 | 0 |
| 10 | 6.0 | 20 | 140 | 0 | 0 | 0 | 0.25 | 0 |
| 11 | 6.0 | 20 | 140 | 0 | 0 | 0 | 0 | 0.5 |
| 12 | 6.0 | 20 | 140 | 0 | 0 | 0 | 0 | 1 |

| | |
|---|---|
| 13 | Tysabri |

TABLE 78
pH, Conductivity, UV and Turbidity measurement for Block 3 at T = 0

| Form | Time Point | Temp. | | UV | Turbidity | |
|---|---|---|---|---|---|---|
| No | (Weeks) | C. ° | pH | mg/mL | Slope (400 to 320 nm) | A.I. |
| 1 | 0 | N/A | 6.08 | 19.75 | −8.64E−06 | 0.56 |
| 2 | 0 | N/A | 6.08 | 19.80 | 2.45E−05 | 0.42 |
| 3 | 0 | N/A | 6.08 | 19.92 | 4.58E−06 | 0.30 |
| 4 | 0 | N/A | 6.07 | 20.09 | 3.64E−06 | 0.30 |
| 5 | 0 | N/A | 6.08 | 20.16 | 6.42E−06 | 0.39 |
| 6 | 0 | N/A | 6.08 | 19.64 | −1.44E−05 | 0.12 |
| 7 | 0 | N/A | 6.06 | 20.26 | 3.98E−06 | 1.00 |
| 8 | 0 | N/A | 6.05 | 19.36 | 5.66E−06 | −0.20 |
| 9 | 0 | N/A | 6.06 | 18.64 | −6.13E−06 | 0.36 |
| 10 | 0 | N/A | 6.05 | 20.10 | −4.14E−06 | −0.23 |
| 11 | 0 | N/A | 6.06 | 20.47 | −6.01E−06 | 0.20 |
| 12 | 0 | N/A | 6.04 | 20.24 | −1.36E−05 | 0.29 |
| 13 | 0 | N/A | 6.27 | 21.46 | −1.62E−05 | −0.64 |

TABLE 79
Visual Inspection for Color and Particles for Block 3 at T = 0

| Form | Time Point | Temp. | Inspection | Inspection |
|---|---|---|---|---|
| No | (Weeks) | C. ° | Color | Particles |
| 1 | 0 | N/A | Clear | Clear |
| 2 | 0 | N/A | Clear | Clear |
| 3 | 0 | N/A | Clear | Clear |
| 4 | 0 | N/A | Clear | Clear |
| 5 | 0 | N/A | Clear | Clear |
| 6 | 0 | N/A | Clear | Clear |
| 7 | 0 | N/A | Clear | Clear |
| 8 | 0 | N/A | Clear | Clear |
| 9 | 0 | N/A | Clear | Clear |
| 10 | 0 | N/A | Clear | Clear |
| 11 | 0 | N/A | Clear | Clear |
| 12 | 0 | N/A | Clear | Clear |
| 13 | 0 | N/A | Clear | Clear |
| 14 | 0 | N/A | Clear | Clear |
| 15 | 0 | N/A | Clear | Clear |
| 16 | 0 | N/A | Clear | Clear |

TABLE 80
Reverse Phase data for Block 3 at T = 0

| | | | Reverse Phase | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 1.16 | 98.84 | 0.00 |
| 2 | 0 | N/A | 1.16 | 98.84 | 0.00 |
| 3 | 0 | N/A | 1.20 | 98.80 | 0.00 |
| 4 | 0 | N/A | 1.18 | 98.82 | 0.00 |
| 5 | 0 | N/A | 1.18 | 98.82 | 0.00 |
| 6 | 0 | N/A | 1.18 | 98.82 | 0.00 |
| 7 | 0 | N/A | 1.18 | 98.82 | 0.00 |
| 8 | 0 | N/A | 1.21 | 98.79 | 0.00 |
| 9 | 0 | N/A | 1.24 | 98.76 | 0.00 |
| 10 | 0 | N/A | 1.19 | 98.81 | 0.00 |
| 11 | 0 | N/A | 1.17 | 98.83 | 0.00 |
| 12 | 0 | N/A | 1.22 | 98.78 | 0.00 |
| 13 | 0 | N/A | 1.23 | 98.77 | 0.00 |

TABLE 81
SEC data for Block 3 at 2 weeks at T = 0

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 0.26 | 98.86 | 0.29 |
| 2 | 0 | N/A | 0.28 | 98.80 | 0.31 |
| 3 | 0 | N/A | 0.29 | 98.78 | 0.33 |
| 4 | 0 | N/A | 0.29 | 98.78 | 0.34 |
| 5 | 0 | N/A | 0.27 | 98.79 | 0.32 |
| 6 | 0 | N/A | 0.29 | 98.78 | 0.36 |
| 7 | 0 | N/A | 0.29 | 98.76 | 0.35 |
| 8 | 0 | N/A | 0.30 | 98.75 | 0.35 |
| 9 | 0 | N/A | | | |
| 10 | 0 | N/A | 0.29 | 98.76 | 0.35 |
| 11 | 0 | N/A | 0.28 | 98.77 | 0.35 |
| 12 | 0 | N/A | 0.30 | 98.75 | 0.35 |
| 13 | 0 | N/A | 0.27 | 98.52 | 0.47 |

TABLE 82
HIC data for Block 3 at 2 weeks at T = 0

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 1.16 | 98.84 | 0.00 |
| 2 | 0 | N/A | 1.16 | 98.84 | 0.00 |
| 3 | 0 | N/A | 1.20 | 98.80 | 0.00 |
| 4 | 0 | N/A | 1.18 | 98.82 | 0.00 |
| 5 | 0 | N/A | 1.18 | 98.82 | 0.00 |
| 6 | 0 | N/A | 1.18 | 98.82 | 0.00 |
| 7 | 0 | N/A | 1.18 | 98.82 | 0.00 |
| 8 | 0 | N/A | 1.21 | 98.79 | 0.00 |
| 9 | 0 | N/A | 1.24 | 98.76 | 0.00 |
| 10 | 0 | N/A | 1.19 | 98.81 | 0.00 |
| 11 | 0 | N/A | 1.17 | 98.83 | 0.00 |
| 12 | 0 | N/A | 1.22 | 98.78 | 0.00 |
| 13 | 0 | N/A | 1.23 | 98.77 | 0.00 |

TABLE 83
CEX data for Block 3 at 2 weeks at T = 0

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 16.22 | 74.17 | 9.61 |
| 2 | 0 | N/A | 16.26 | 74.17 | 9.57 |
| 3 | 0 | N/A | 16.28 | 74.32 | 9.40 |
| 4 | 0 | N/A | 16.41 | 74.04 | 9.55 |
| 5 | 0 | N/A | 16.45 | 73.95 | 9.60 |
| 6 | 0 | N/A | 16.42 | 74.07 | 9.51 |
| 7 | 0 | N/A | 16.48 | 74.03 | 9.49 |
| 8 | 0 | N/A | 16.59 | 73.66 | 9.75 |
| 9 | 0 | N/A | 16.73 | 73.56 | 9.71 |
| 10 | 0 | N/A | 16.69 | 73.57 | 9.74 |
| 11 | 0 | N/A | 16.43 | 73.97 | 9.60 |
| 12 | 0 | N/A | 16.66 | 73.55 | 9.79 |
| 13 | 0 | N/A | 17.01 | 73.34 | 9.65 |

TABLE 84
Total Particle Count by flow imaging for Block 3 samples at T = 0

| Form | Time Point | Temp. | Total Particle Count (counts/mL) | | | | |
|---|---|---|---|---|---|---|---|
| No | (Weeks) | C. ° | All | 2 uM | 5 uM | 10 uM | 25 uM |
| 1 | 0 | N/A | 36088 | 23502 | 13378 | 5626 | 670 |
| 2 | 0 | N/A | 9708 | 3082 | 782 | 183 | 37 |
| 3 | 0 | N/A | 1285 | 246 | 80 | 37 | 5 |
| 4 | 0 | N/A | 28772 | 2766 | 476 | 135 | 18 |
| 5 | 0 | N/A | 5259 | 1972 | 641 | 246 | 40 |
| 6 | 0 | N/A | 22469 | 6442 | 1293 | 343 | 61 |
| 7 | 0 | N/A | 28059 | 5709 | 799 | 151 | 0 |
| 8 | 0 | N/A | 19609 | 2802 | 450 | 79 | 20 |
| 9 | 0 | N/A | 16692 | 1842 | 422 | 129 | 59 |
| 10 | 0 | N/A | 5675 | 1744 | 526 | 197 | 16 |
| 11 | 0 | N/A | 26842 | 8050 | 1994 | 468 | 16 |
| 12 | 0 | N/A | 79657 | 24713 | 6261 | 1656 | 127 |
| 13 | 0 | N/A | 32984 | 9591 | 1863 | 314 | 26 |

TABLE 86
UV and Turbidity measurement for Block 3 at 2 weeks, 40 C. °

| Form | Time Point | Temp. | UV | Turbidity | |
|---|---|---|---|---|---|
| No | (Weeks) | C. ° | mg/mL | Slope (400 to 320 nm) | A.I. |
| 1 | 2 | 40 | 19.83 | −1.89E−05 | 0.63 |
| 2 | 2 | 40 | 20.07 | −2.21E−05 | 0.81 |
| 3 | 2 | 40 | 20.24 | −3.15E−05 | 0.66 |
| 4 | 2 | 40 | 20.16 | −4.10E−06 | 0.81 |
| 5 | 2 | 40 | 20.28 | −2.22E−05 | 0.34 |
| 6 | 2 | 40 | 20.28 | −2.13E−05 | 0.11 |
| 7 | 2 | 40 | 20.52 | −2.31E−05 | −0.09 |
| 8 | 2 | 40 | 20.05 | −1.49E−05 | 0.85 |
| 9 | 2 | 40 | 18.49 | −3.45E−05 | 0.52 |
| 10 | 2 | 40 | 19.88 | −2.05E−05 | 0.27 |
| 11 | 2 | 40 | 20.52 | −2.38E−05 | −0.04 |
| 12 | 2 | 40 | 19.81 | −3.46E−05 | 0.01 |
| 13 | 2 | 40 | 21.86 | −4.47E−05 | 0.90 |

TABLE 87
Visual Inspection for Color and Particles
for Block 3 at 2 weeks, 40 C. °

| Form | Time Point | Temp. | Inspection | Inspection |
|---|---|---|---|---|
| No | (Weeks) | C. ° | Color | Particles |
| 1 | 2 | 40 | Clear | Clear |
| 2 | 2 | 40 | Clear | Clear |
| 3 | 2 | 40 | Clear | Clear |
| 4 | 2 | 40 | Clear | Clear |
| 5 | 2 | 40 | Clear | Clear |
| 6 | 2 | 40 | Clear | Clear |
| 7 | 2 | 40 | Clear | Clear |
| 8 | 2 | 40 | Clear | Clear |
| 9 | 2 | 40 | Clear | Clear |
| 10 | 2 | 40 | Clear | Clear |
| 11 | 2 | 40 | Clear | Clear |
| 12 | 2 | 40 | Clear | Clear |
| 13 | 2 | 40 | Clear | Clear |
| 14 | 2 | 40 | Clear | Clear |
| 15 | 2 | 40 | Clear | Clear |
| 16 | 2 | 40 | Clear | Clear |

TABLE 88
Reverse Phase data for Block 3 at 2 weeks, 40 C. °

| | | | RP | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 1.40 | 98.60 | 0.00 |
| 2 | 2 | 40 | 1.45 | 98.55 | 0.00 |
| 3 | 2 | 40 | 1.32 | 98.68 | 0.00 |
| 4 | 2 | 40 | 1.39 | 98.61 | 0.00 |
| 5 | 2 | 40 | 1.38 | 98.62 | 0.00 |
| 6 | 2 | 40 | 1.37 | 98.63 | 0.00 |
| 7 | 2 | 40 | 1.40 | 98.60 | 0.00 |
| 8 | 2 | 40 | 1.40 | 98.60 | 0.00 |
| 9 | 2 | 40 | 1.48 | 98.52 | 0.00 |
| 10 | 2 | 40 | 1.42 | 98.58 | 0.00 |
| 11 | 2 | 40 | 1.32 | 98.68 | 0.00 |
| 12 | 2 | 40 | 1.30 | 98.70 | 0.00 |
| 13 | 2 | 40 | 1.45 | 98.55 | 0.00 |

TABLE 89
SEC data for Block 3 at 2 weeks, 40 C. °

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 0.23 | 98.58 | 1.19 |
| 2 | 2 | 40 | 0.23 | 98.57 | 1.19 |
| 3 | 2 | 40 | 0.25 | 98.62 | 1.13 |
| 4 | 2 | 40 | 0.26 | 98.56 | 1.18 |
| 5 | 2 | 40 | 0.29 | 98.58 | 1.12 |
| 6 | 2 | 40 | 0.30 | 98.57 | 1.13 |
| 7 | 2 | 40 | 0.24 | 98.60 | 1.16 |
| 8 | 2 | 40 | 0.23 | 98.62 | 1.15 |
| 9 | 2 | 40 | 0.24 | 98.52 | 1.25 |
| 10 | 2 | 40 | 0.26 | 98.52 | 1.20 |
| 11 | 2 | 40 | 0.25 | 98.61 | 1.14 |
| 12 | 2 | 40 | 0.27 | 98.64 | 1.08 |
| 13 | 2 | 40 | 0.33 | 98.50 | 1.17 |

TABLE 90
HIC data for Block 3 at 2 weeks, 40 C. °

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 9.52 | 90.48 | 0.00 |
| 2 | 2 | 40 | 8.75 | 91.25 | 0.00 |
| 3 | 2 | 40 | 10.67 | 89.33 | 0.00 |
| 4 | 2 | 40 | 8.79 | 91.21 | 0.00 |
| 5 | 2 | 40 | 13.76 | 86.24 | 0.00 |
| 6 | 2 | 40 | 23.23 | 76.77 | 0.00 |
| 7 | 2 | 40 | 8.62 | 91.38 | 0.00 |
| 8 | 2 | 40 | 9.24 | 90.76 | 0.00 |
| 9 | 2 | 40 | 8.82 | 91.18 | 0.00 |
| 10 | 2 | 40 | 8.72 | 91.28 | 0.00 |
| 11 | 2 | 40 | 11.38 | 88.62 | 0.00 |
| 12 | 2 | 40 | 15.36 | 84.64 | 0.00 |
| 13 | 2 | 40 | 9.24 | 90.76 | 0.00 |

TABLE 91
CEX data for Block 3 at 2 weeks, 40 C. °

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 19.60 | 70.52 | 9.88 |
| 2 | 2 | 40 | 19.61 | 70.67 | 9.72 |
| 3 | 2 | 40 | 19.98 | 70.26 | 9.76 |
| 4 | 2 | 40 | 19.31 | 71.05 | 9.64 |
| 5 | 2 | 40 | 19.58 | 70.74 | 9.68 |
| 6 | 2 | 40 | 19.30 | 70.87 | 9.83 |
| 7 | 2 | 40 | 19.10 | 71.25 | 9.65 |
| 8 | 2 | 40 | 19.81 | 70.55 | 9.64 |
| 9 | 2 | 40 | 19.24 | 71.43 | 9.33 |
| 10 | 2 | 40 | 19.76 | 70.56 | 9.68 |
| 11 | 2 | 40 | 19.65 | 70.76 | 9.59 |
| 12 | 2 | 40 | | | |
| 13 | 2 | 40 | | | |

TABLE 92
UV and Turbidity measurement for Block 3 at 4 weeks, 25 C. °

| Form | Time Point | Temp. | UV | Turbidity | |
|---|---|---|---|---|---|
| No | (Weeks) | C. ° | mg/mL | Slope (400 to 320 nm) | A.I. |
| 1 | 4 | 25 | 20.02 | 4.10E−06 | 0.24 |
| 2 | 4 | 25 | 20.13 | −1.88E−05 | 0.24 |
| 3 | 4 | 25 | 20.23 | 1.63E−06 | −0.15 |
| 4 | 4 | 25 | 20.10 | 3.72E−06 | 0.25 |
| 5 | 4 | 25 | 20.68 | −2.05E−05 | 1.39 |
| 6 | 4 | 25 | 20.37 | 2.32E−05 | 0.12 |
| 7 | 4 | 25 | 20.37 | −8.65E−06 | −0.11 |
| 8 | 4 | 25 | 20.14 | 4.89E−06 | 0.12 |
| 9 | 4 | 25 | 18.89 | 3.87E−06 | −0.06 |
| 10 | 4 | 25 | 20.37 | 4.53E−06 | −0.24 |
| 11 | 4 | 25 | 20.76 | −7.03E−06 | −0.48 |
| 12 | 4 | 25 | 20.33 | −1.18E−05 | −0.10 |
| 13 | 4 | 25 | 21.96 | 6.84E−06 | 0.06 |

TABLE 93
Visual Inspection for Color and Particles
for Block 3 at 4 weeks, 25 C. °

| Form | Time Point | Temp. | Inspection | Inspection |
|---|---|---|---|---|
| No | (Weeks) | C. ° | Color | Particles |
| 1 | 4 | 25 | Clear | Clear |
| 2 | 4 | 25 | Clear | Clear |
| 3 | 4 | 25 | Clear | Clear |
| 4 | 4 | 25 | Clear | Clear |
| 5 | 4 | 25 | Clear | Clear |
| 6 | 4 | 25 | Clear | Clear |
| 7 | 4 | 25 | Clear | Clear |
| 8 | 4 | 25 | Clear | Clear |
| 9 | 4 | 25 | Clear | Clear |
| 10 | 4 | 25 | Clear | Clear |
| 11 | 4 | 25 | Clear | Clear |
| 12 | 4 | 25 | Clear | Clear |
| 13 | 4 | 25 | Clear | Clear |
| 14 | 4 | 25 | Clear | Clear |
| 15 | 4 | 25 | Clear | Clear |
| 16 | 4 | 25 | Clear | Clear |

TABLE 94
Reverse Phase data for Block 3 at 4 weeks, 25 C. °

| | | | RP | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 1.11 | 98.89 | 0.00 |
| 2 | 4 | 25 | 1.17 | 98.83 | 0.00 |
| 3 | 4 | 25 | 1.09 | 98.91 | 0.00 |
| 4 | 4 | 25 | 1.17 | 98.83 | 0.00 |
| 5 | 4 | 25 | 1.11 | 98.89 | 0.00 |
| 6 | 4 | 25 | 1.15 | 98.85 | 0.00 |
| 7 | 4 | 25 | 1.13 | 98.87 | 0.00 |
| 8 | 4 | 25 | 1.15 | 98.85 | 0.00 |
| 9 | 4 | 25 | 1.15 | 98.85 | 0.00 |
| 10 | 4 | 25 | 1.18 | 98.82 | 0.00 |
| 11 | 4 | 25 | 1.12 | 98.88 | 0.00 |
| 12 | 4 | 25 | 1.19 | 98.81 | 0.00 |
| 13 | 4 | 25 | 1.21 | 98.79 | 0.00 |

TABLE 95
SEC data for Block 3 at 2 weeks at 4 weeks, 25 C. °

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 0.28 | 98.83 | 0.89 |
| 2 | 4 | 25 | 0.25 | 98.84 | 0.91 |
| 3 | 4 | 25 | 0.25 | 98.85 | 0.90 |
| 4 | 4 | 25 | 0.28 | 98.80 | 0.92 |
| 5 | 4 | 25 | 0.29 | 98.80 | 0.91 |
| 6 | 4 | 25 | 0.28 | 98.77 | 0.96 |
| 7 | 4 | 25 | 0.29 | 98.79 | 0.92 |
| 8 | 4 | 25 | 0.28 | 98.78 | 0.94 |
| 9 | 4 | 25 | 0.28 | 98.78 | 0.94 |
| 10 | 4 | 25 | 0.28 | 98.81 | 0.91 |
| 11 | 4 | 25 | 0.28 | 98.80 | 0.92 |
| 12 | 4 | 25 | 0.29 | 98.80 | 0.90 |
| 13 | 4 | 25 | 0.31 | 98.73 | 0.95 |

TABLE 96
HIC data for Block 3 at 2 weeks at 4 weeks, 25 C. °

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 8.30 | 91.70 | 0.00 |
| 2 | 4 | 25 | 7.56 | 92.44 | 0.00 |
| 3 | 4 | 25 | 8.07 | 91.93 | 0.00 |
| 4 | 4 | 25 | 7.55 | 92.45 | 0.00 |
| 5 | 4 | 25 | 7.96 | 92.04 | 0.00 |
| 6 | 4 | 25 | 9.25 | 90.75 | 0.00 |
| 7 | 4 | 25 | 7.76 | 92.24 | 0.00 |
| 8 | 4 | 25 | 7.97 | 92.03 | 0.00 |
| 9 | 4 | 25 | 7.71 | 92.29 | 0.00 |
| 10 | 4 | 25 | 7.94 | 92.06 | 0.00 |
| 11 | 4 | 25 | 8.01 | 91.99 | 0.00 |
| 12 | 4 | 25 | 9.44 | 90.56 | 0.00 |
| 13 | 4 | 25 | 7.94 | 92.06 | 0.00 |

TABLE 97
CEX data for Block 3 at 2 weeks at 4 weeks, 25 C. °

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 25.80 | 63.83 | 10.37 |
| 2 | 4 | 25 | 25.30 | 64.50 | 10.21 |
| 3 | 4 | 25 | 25.32 | 64.63 | 10.05 |
| 4 | 4 | 25 | 24.77 | 65.28 | 9.95 |
| 5 | 4 | 25 | 24.44 | 65.55 | 10.01 |
| 6 | 4 | 25 | 24.99 | 64.93 | 10.08 |
| 7 | 4 | 25 | 25.54 | 64.32 | 10.14 |
| 8 | 4 | 25 | 24.62 | 65.32 | 10.07 |
| 9 | 4 | 25 | 24.98 | 64.98 | 10.04 |
| 10 | 4 | 25 | 25.20 | 64.60 | 10.19 |
| 11 | 4 | 25 | 25.15 | 65.06 | 9.79 |
| 12 | 4 | 25 | 25.17 | 64.93 | 9.89 |
| 13 | 4 | 25 | 27.72 | 63.01 | 9.27 |

TABLE 98
Total Particle Count by flow imaging for Block 3 samples after 4 weeks at 25 C. °

| Form | Time Point | Temp. | Total Particle Count (counts/mL) | | | | |
|---|---|---|---|---|---|---|---|
| No | (Weeks) | C. ° | All | 2 uM | 5 uM | 10 uM | 25 uM |
| 1 | 4 | 25 | 265378 | 126088 | 45778 | 12868 | 1014 |
| 2 | 4 | 25 | 1005 | 197 | 49 | 21 | 14 |
| 3 | 4 | 25 | 8169 | 1804 | 530 | 224 | 30 |
| 4 | 4 | 25 | 2489 | 426 | 36 | 29 | 7 |
| 5 | 4 | 25 | 2157 | 466 | 115 | 25 | 13 |
| 6 | 4 | 25 | 2553 | 439 | 99 | 26 | 10 |
| 7 | 4 | 25 | 41169 | 9241 | 1987 | 484 | 54 |
| 8 | 4 | 25 | 47498 | 11831 | 2801 | 632 | 39 |
| 9 | 4 | 25 | 4501 | 1324 | 359 | 107 | 25 |
| 10 | 4 | 25 | 1938 | 691 | 92 | 43 | 37 |
| 11 | 4 | 25 | 155182 | 43122 | 12699 | 3768 | 488 |
| 12 | 4 | 25 | 93295 | 38506 | 13129 | 3859 | 208 |
| 13 | 4 | 25 | 149219 | 65109 | 21723 | 6087 | 344 |

In general, the exemplary formulations tested in Study 3 have good chemical stability and are suitable for long-term storage, as determined by RP HPLC and CEX. However, some differences are apparent. For example, the initial levels of SVPs is greater for Formulation 1 (no surfactant) and Formulation 12 (PEG only) (Table 84). As samples are stored, the stability measured by HIC shows some distinct differences. The formulations with PEG or with progressively higher levels of PS 20 show a sizable loss of the main peak (Tables 90 and 96). This may reflect some chemical degradation of PS 20 at 40° C. It is known that PS 20 is prone to hydrolysis, releasing free fatty acids into solution. Otherwise, all of the formulations perform well and are comparable to the marketed formulation. The levels of SVPs increase upon storage at elevated temperature, with the surfactant-free formulation showing the greatest numbers of particles. Of the excipients tested here, Pluronic F-68 and PEG 3350 appear to be the least protective during storage.

Agitation Study

An agitation study was performed with some of the formulations from Study 3. Samples were pulled and analyzed after 24 or 48 hours of agitation on an orbital shaker. No changes were seen by RP HPLC or CEX, indicating that agitation does not cause chemical damage. There is also no loss by SEC. By HIC, the surfactant-free formulation appears slightly less stable (Table 110). Overall, all of the surfactants evaluated here provided acceptable protection against agitation-induced damage.

TABLE 100
UV and Turbidity measurement for Agitation Study for Block 3

| Form | UV mg/mL | UV mg/mL | UV mg/mL | Slope | Slope 24 | Slope 48 | A.I. | A.I. | A.I. |
|---|---|---|---|---|---|---|---|---|---|
| No | Control | 24 Hours | 48 Hours | Control | Hours | Hours | Control | 24 Hours | 48 Hours |
| 1 | 20.25 | | 20.72 | −4.21E−06 | | −1.51E−05 | −0.36 | | −0.20 |
| 2 | 20.03 | | 20.44 | −1.98E−05 | | 5.31E−07 | −0.30 | | −0.60 |
| 3 | 20.33 | | 20.22 | −3.41E−05 | | −1.07E−05 | −0.09 | | −0.40 |
| 4 | 19.90 | | 20.69 | −2.03E−05 | | −1.52E−05 | −0.44 | | −0.25 |
| 5 | 20.10 | | 20.66 | 3.07E−06 | | −1.78E−05 | −0.51 | | −0.44 |
| 6 | 20.23 | | 19.95 | −2.00E−05 | | −1.30E−05 | −0.49 | | −0.62 |
| 7 | 19.88 | | 20.73 | −1.59E−05 | | −1.76E−05 | −0.66 | | 0.25 |
| 8 | 19.69 | | 20.13 | −1.07E−05 | | −2.13E−05 | −0.69 | | −0.43 |
| 9 | 18.80 | | 18.97 | −8.56E−07 | | −8.83E−06 | −0.70 | | −0.48 |
| 10 | 20.45 | | 21.53 | −2.26E−05 | | −1.82E−05 | 0.09 | | −0.66 |
| 11 | 20.80 | | 21.44 | −1.30E−05 | | −8.96E−06 | 0.37 | | −0.15 |
| 12 | 20.81 | | 21.22 | −3.36E−05 | | −1.53E−05 | 2.08 | | 0.64 |
| 13 | 21.58 | | 22.68 | −1.45E−05 | | 1.65E−04 | −0.42 | | 0.35 |

TABLE 101
Visual Inspection for Particles for Agitation Study for Block 3

| Form | Visual | Visual 24 | Visual 48 | Visual | Visual | Visual 48 | Visual | Visual | Visual 48 |
|---|---|---|---|---|---|---|---|---|---|
| No | Control | Hours | Hours | Control | 24 Hours | Hours | Control | 24 Hours | Hours |
| 1 | Clear | Clear | Clear | Clear | Clear | Clear | Clear | Clear | Clear |
| 2 | Clear | Clear | Clear | | | | Clear | Clear | Clear |
| 3 | Clear | Clear | Clear | Clear | Clear | Clear | Clear | Clear | Clear |
| 4 | Clear | Clear | Clear | | | | Clear | Clear | Clear |
| 5 | Clear | Clear | Clear | | | | Clear | Clear | Clear |
| 6 | Clear | Clear | Clear | | | | Clear | Clear | Clear |
| 7 | Clear | Clear | Clear | | | | Clear | Clear | Clear |
| 8 | Clear | Clear | Clear | Clear | Clear | Clear | Clear | Clear | Clear |
| 9 | Clear | Clear | Clear | | | | Clear | Clear | Clear |
| 10 | Clear | Clear | Clear | | | | Clear | Clear | Clear |
| 11 | Clear | Clear | Clear | | | | Clear | Clear | Clear |
| 12 | Clear | Clear | Clear | | | | Clear | Clear | Clear |
| 13 | Clear | Clear | Clear | | | | Clear | Clear | Clear |

TABLE 102
Reverse Phase Control data for Agitation Study for Block 3

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 0 | 25 | 1.05 | 98.95 | 0.00 |
| 2 | 0 | 25 | 1.02 | 98.98 | 0.00 |
| 3 | 0 | 25 | 1.08 | 98.92 | 0.00 |
| 4 | 0 | 25 | 1.07 | 98.93 | 0.00 |
| 5 | 0 | 25 | 1.08 | 98.92 | 0.00 |
| 6 | 0 | 25 | 1.08 | 98.92 | 0.00 |
| 7 | 0 | 25 | 1.08 | 98.92 | 0.00 |
| 8 | 0 | 25 | 1.04 | 98.96 | 0.00 |
| 9 | 0 | 25 | 1.11 | 98.89 | 0.00 |
| 10 | 0 | 25 | 1.03 | 98.97 | 0.00 |
| 11 | 0 | 25 | 1.05 | 98.95 | 0.00 |
| 12 | 0 | 25 | 1.04 | 98.96 | 0.00 |
| 13 | 0 | 25 | 1.02 | 98.98 | 0.00 |

TABLE 103
Reverse Phase data for 24 Hours Agitation Study for Block 3

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 24 | 25 | 0.99 | 99.01 | 0.00 |
| 3 | 24 | 25 | 1.05 | 98.95 | 0.00 |
| 8 | 24 | 25 | 1.08 | 98.92 | 0.00 |

TABLE 104
Reverse Phase data for 48 Hours Agitation Study for Block 3

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 48 | 25 | 1.00 | 99.00 | 0.00 |
| 2 | 48 | 25 | 1.05 | 98.95 | 0.00 |
| 3 | 48 | 25 | 1.09 | 98.91 | 0.00 |
| 4 | 48 | 25 | 1.06 | 98.94 | 0.00 |
| 5 | 48 | 25 | 1.09 | 98.91 | 0.00 |
| 6 | 48 | 25 | 1.09 | 98.91 | 0.00 |
| 7 | 48 | 25 | 1.11 | 98.89 | 0.00 |
| 8 | 48 | 25 | 1.13 | 98.87 | 0.00 |
| 9 | 48 | 25 | 1.12 | 98.88 | 0.00 |
| 10 | 48 | 25 | 1.08 | 98.92 | 0.00 |
| 11 | 48 | 25 | 1.08 | 98.92 | 0.00 |
| 12 | 48 | 25 | 0.98 | 99.02 | 0.00 |
| 13 | 48 | 25 | 1.05 | 98.95 | 0.00 |

TABLE 105
SEC Control data for Agitation Study for Block 3

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 0 | 25 | 0.27 | 98.83 | 0.91 |
| 2 | 0 | 25 | 0.27 | 98.84 | 0.88 |
| 3 | 0 | 25 | 0.28 | 98.83 | 0.88 |
| 4 | 0 | 25 | 0.28 | 98.86 | 0.87 |
| 5 | 0 | 25 | 0.28 | 98.82 | 0.90 |
| 6 | 0 | 25 | 0.29 | 98.85 | 0.86 |
| 7 | 0 | 25 | 0.29 | 98.81 | 0.90 |
| 8 | 0 | 25 | 0.29 | 98.82 | 0.89 |
| 9 | 0 | 25 | 0.29 | 98.80 | 0.90 |
| 10 | 0 | 25 | 0.29 | 98.82 | 0.89 |
| 11 | 0 | 25 | 0.28 | 98.85 | 0.86 |
| 12 | 0 | 25 | 0.29 | 98.84 | 0.87 |
| 13 | 0 | 25 | 0.25 | 98.84 | 0.91 |

TABLE 106
SEC data for 24 Hours Agitation Study for Block 3

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 24 | 25 | 0.27 | 98.87 | 0.86 |
| 3 | 24 | 25 | 0.28 | 98.85 | 0.87 |
| 8 | 24 | 25 | 0.27 | 98.86 | 0.86 |

TABLE 107
SEC data for 48 Hours Agitation Study for Block 3

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 48 | 25 | 0.27 | 98.82 | 0.91 |
| 2 | 48 | 25 | 0.29 | 98.82 | 0.88 |
| 3 | 48 | 25 | 0.28 | 98.81 | 0.91 |
| 4 | 48 | 25 | 0.30 | 98.83 | 0.87 |
| 5 | 48 | 25 | 0.31 | 98.77 | 0.92 |
| 6 | 48 | 25 | 0.28 | 98.81 | 0.91 |
| 7 | 48 | 25 | 0.29 | 98.80 | 0.91 |
| 8 | 48 | 25 | 0.29 | 98.85 | 0.86 |
| 9 | 48 | 25 | 0.29 | 98.80 | 0.91 |
| 10 | 48 | 25 | 0.27 | 98.83 | 0.90 |
| 11 | 48 | 25 | 0.29 | 98.81 | 0.90 |
| 12 | 48 | 25 | 0.28 | 98.85 | 0.88 |
| 13 | 48 | 25 | 0.27 | 98.77 | 0.95 |

TABLE 108
HIC Control data for Agitation Study for Block 3

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 0 | 25 | 8.29 | 91.71 | 0.00 |
| 2 | 0 | 25 | 7.54 | 92.46 | 0.00 |
| 3 | 0 | 25 | 7.80 | 92.20 | 0.00 |
| 4 | 0 | 25 | 7.54 | 92.46 | 0.00 |
| 5 | 0 | 25 | 7.24 | 92.76 | 0.00 |
| 6 | 0 | 25 | 7.39 | 92.61 | 0.00 |
| 7 | 0 | 25 | 7.15 | 92.85 | 0.00 |
| 8 | 0 | 25 | 6.83 | 93.17 | 0.00 |
| 9 | 0 | 25 | 7.18 | 92.82 | 0.00 |
| 10 | 0 | 25 | 7.14 | 92.86 | 0.00 |
| 11 | 0 | 25 | 7.04 | 92.96 | 0.00 |
| 12 | 0 | 25 | 7.72 | 92.28 | 0.00 |
| 13 | 0 | 25 | 7.26 | 92.74 | 0.00 |

TABLE 109
HIC data for 24 Hours Agitation Study for Block 3

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 24 | 25 | 8.02 | 91.98 | 0.00 |
| 3 | 24 | 25 | 7.47 | 92.53 | 0.00 |
| 8 | 24 | 25 | 7.23 | 92.77 | 0.00 |

TABLE 110
HIC data for 48 Hours Agitation Study for Block 3

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 48 | 25 | 8.07 | 91.93 | 0.00 |
| 2 | 48 | 25 | 7.55 | 92.45 | 0.00 |
| 3 | 48 | 25 | 7.77 | 92.23 | 0.00 |
| 4 | 48 | 25 | 7.05 | 92.95 | 0.00 |
| 5 | 48 | 25 | 7.24 | 92.76 | 0.00 |
| 6 | 48 | 25 | 7.22 | 92.78 | 0.00 |
| 7 | 48 | 25 | 7.10 | 92.90 | 0.00 |
| 8 | 48 | 25 | 7.11 | 92.89 | 0.00 |
| 9 | 48 | 25 | 7.12 | 92.88 | 0.00 |
| 10 | 48 | 25 | 7.25 | 92.75 | 0.00 |
| 11 | 48 | 25 | 7.35 | 92.65 | 0.00 |
| 12 | 48 | 25 | 7.51 | 92.49 | 0.00 |
| 13 | 48 | 25 | 7.39 | 92.61 | 0.00 |

TABLE 111
CEX Control data for Agitation Study for Block 3

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 0 | 25 | 17.02 | 73.78 | 9.19 |
| 2 | 0 | 25 | 17.22 | 73.79 | 8.99 |
| 3 | 0 | 25 | 16.71 | 74.32 | 8.97 |
| 4 | 0 | 25 | 17.00 | 73.88 | 9.13 |
| 5 | 0 | 25 | 17.13 | 73.72 | 9.15 |
| 6 | 0 | 25 | 17.05 | 73.95 | 9.00 |
| 7 | 0 | 25 | 16.80 | 74.04 | 9.16 |
| 8 | 0 | 25 | 16.97 | 73.79 | 9.23 |
| 9 | 0 | 25 | 17.04 | 73.93 | 9.03 |
| 10 | 0 | 25 | 16.93 | 73.76 | 9.31 |
| 11 | 0 | 25 | 16.82 | 74.09 | 9.09 |
| 12 | 0 | 25 | 17.27 | 73.50 | 9.53 |
| 13 | 0 | 25 | 17.45 | 73.43 | 9.12 |

TABLE 112
CEX data for 24 Hours Agitation Study for Block 3

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 24 | 25 | 16.44 | 74.47 | 9.10 |
| 3 | 24 | 25 | 17.05 | 73.74 | 9.21 |
| 8 | 24 | 25 | 17.06 | 73.68 | 9.26 |

TABLE 113
CEX data for 48 Hours Agitation Study for Block 3

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Hours) | C. ° | (%) | (%) | (%) |
| 1 | 48 | 25 | 17.21 | 73.54 | 9.25 |
| 2 | 48 | 25 | 17.05 | 73.75 | 9.20 |
| 3 | 48 | 25 | 17.17 | 73.82 | 9.01 |
| 4 | 48 | 25 | 17.12 | 73.83 | 9.05 |
| 5 | 48 | 25 | 17.26 | 73.51 | 9.23 |
| 6 | 48 | 25 | 17.03 | 73.80 | 9.17 |
| 7 | 48 | 25 | 16.88 | 73.87 | 9.25 |
| 8 | 48 | 25 | 17.13 | 73.52 | 9.35 |
| 9 | 48 | 25 | 16.73 | 73.80 | 9.48 |
| 10 | 48 | 25 | 16.90 | 73.74 | 9.36 |
| 11 | 48 | 25 | 17.26 | 73.32 | 9.69 |
| 12 | 48 | 25 | 17.28 | 73.58 | 9.14 |
| 13 | 48 | 25 | 17.58 | 73.29 | 9.13 |

TABLE 114
Total Particle Count by flow imaging
for Block 3 samples after agitation

| Form | Time Point | Total Particle Count (counts/mL) | | | | |
|---|---|---|---|---|---|---|
| No | (Hours) | All | 2 uM | 5 uM | 10 uM | 25 uM |
| 1 | Control | 64185 | 42224 | 24954 | 12075 | 1629 |
| 2 | Control | 8676 | 1321 | 336 | 164 | 31 |
| 8 | Control | 298195 | 69786 | 24677 | 8390 | 1086 |
| 1 | 48 | 70551 | 45807 | 25679 | 11053 | 1110 |
| 2 | 48 | 1995 | 646 | 227 | 128 | 36 |
| 8 | 48 | 67654 | 18604 | 4619 | 1008 | 20 |

Upon agitation, PS 80 protects significantly better than Pluronic F-68 against SVP formation, but both reduce SVP levels compared to the surfactant-free formulation.

Freeze-thaw studies were also conducted with some Block 3 formulations, as summarized in Tables 116 through 121. Of the four formulations evaluated here, there were no notable differences seen by SEC or CEX, but formulations containing surfactants fared better by HIC than Formulation 1, which has no surfactant present (Table 119). The formation of SVPs was also suppressed for these same formulations compared to Formulation 1 (Table 121).

TABLE 116
Study Design for the Block 3 Freeze Thaw

| Form | | Histidine | NaCl | PS 80 | PS 20 | F-68 | DDM | PEG 3350 |
|---|---|---|---|---|---|---|---|---|
| No. | pH | (mM) | (mM) | (%) | (%) | (%) | (%) | (%) |
| 1 | 6.0 | 20 | 140 | 0 | 0 | 0 | 0 | 0 |
| 2 | 6.0 | 20 | 140 | 0.01 | 0 | 0 | 0 | 0 |
| 3 | 6.0 | 20 | 140 | 0.02 | 0 | 0 | 0 | 0 |
| 8 | 6.0 | 20 | 140 | 0 | 0 | 0.2 | 0 | 0 |

TABLE 117
UV and Turbidity measurement for Block 3
Freeze Thaw Studies after 5 freeze thaws

| Form | Time Point | Temp. | UV | Turbidity | |
|---|---|---|---|---|---|
| No | (Weeks) | C. ° | mg/mL | Slope (400 to 320 nm) | A.I. |
| 1 | 8 | −20 | 20.18 | −3.82E−06 | −0.55 |
| 2 | 8 | −20 | 20.27 | −6.30E−06 | −0.82 |
| 3 | 8 | −20 | 20.27 | −9.56E−06 | −0.61 |
| 8 | 8 | −20 | 19.71 | −7.64E−06 | −0.57 |

TABLE 118
SEC data for Block 3 samples after five F/T cycles

| | Control | | | 3 Freeze Thaws | | | 5 Freeze Thaws | | |
|---|---|---|---|---|---|---|---|---|---|
| Form | Before | | After | Before | | After | Before | | After |
| No | Monomer | Monomer | Monomer | Monomer | Monomer | Monomer | Monomer | Monomer | Monomer |
| 1 | 0.26 | 98.86 | 0.29 | 0.20 | 98.87 | 0.93 | 0.34 | 98.71 | 0.94 |
| 2 | 0.28 | 98.8 | 0.31 | 0.19 | 98.87 | 0.94 | N/A | N/A | N/A |
| 3 | 0.29 | 98.78 | 0.33 | 0.20 | 98.86 | 0.95 | 0.31 | 98.69 | 1.00 |
| 8 | 0.3 | 98.75 | 0.35 | 0.25 | 98.81 | 0.94 | 0.30 | 98.71 | 0.99 |

TABLE 119
HIC data for Block 3 samples after five F/T cycles

| | Control | | | 3 Freeze Thaws | | | 5 Freeze Thaws | | |
|---|---|---|---|---|---|---|---|---|---|
| Form | Before | | After | Before | | After | Before | | After |
| No | Monomer | Monomer | Monomer | Monomer | Monomer | Monomer | Monomer | Monomer | Monomer |
| 1 | 0.26 | 98.86 | 0.29 | 7.99 | 92.01 | 0.00 | 9.24 | 90.76 | 0.00 |
| 2 | 0.28 | 98.80 | 0.31 | N/A | N/A | N/A | N/A | N/A | N/A |
| 3 | 0.29 | 98.78 | 0.33 | 7.77 | 92.23 | 0.00 | 8.44 | 91.56 | 0.00 |
| 8 | 0.30 | 98.75 | 0.35 | 7.90 | 92.10 | 0.00 | 8.27 | 91.73 | 0.00 |

TABLE 120
CEX data for Block 3 samples after five F/T cycles

| Form | Control | | | 5 Freeze Thaws | | |
|---|---|---|---|---|---|---|
| No | Before Monomer | Monomer | After Monomer | Before Monomer | Monomer | After Monomer |
| 1 | 16.22 | 74.17 | 9.61 | 17.85 | 72.08 | 10.07 |
| 2 | 16.26 | 74.17 | 9.57 | N/A | N/A | N/A |
| 3 | 16.28 | 74.32 | 9.4 | 17.93 | 71.87 | 10.21 |
| 8 | 16.59 | 73.66 | 9.75 | 18.20 | 71.54 | 10.26 |

TABLE 121
Total Particle Count by flow imaging for Block 3 samples after five F/T cycles

| | Total Particle Count (counts/mL) | | | | | | | | | |
|---|---|---|---|---|---|---|---|---|---|---|
| Form | Control (counts/mL) | | | | | 5 Freeze Thaws (counts/mL) | | | | |
| No | All | ≥2 uM | ≥5 uM | ≥10 uM | ≥25 uM | All | ≥2 uM | ≥5 uM | ≥10 uM | ≥25 uM |
| 1 | 36088 | 23502 | 13378 | 5626 | 670 | 154189 | 97910 | 50278 | 19423 | 1505 |
| 2 | 9708 | 3082 | 782 | 183 | 37 | N/A | N/A | N/A | N/A | N/A |
| 3 | 1285 | 246 | 80 | 37 | 5 | 155011 | 45236 | 8733 | 1810 | 226 |
| 8 | 19609 | 2802 | 450 | 79 | 20 | 71415 | 23077 | 6016 | 1411 | 177 |

### Example 5 (Block or Study 4)—Twelve Selected Natalizumab Formulations

The final block of studies examined 12 selected natalizumab formulations. For the first time, some were at concentrations less than 20 mg/ml. Varying amounts of NaCl were added to formulations containing polysaccharides. Formulation 1 was the same composition as Formulation 2 from Study 2 and served as a control. All of the formulations contained 0.02% PS 80. The design is found in Table 123. Samples were stored at 40° C. for two weeks or four weeks at 25° C.

TABLE 123
Study 4 Design

| Form | | protein | His | Acetate | Sucrose | Proline | Trehalose | NaCl | |
|---|---|---|---|---|---|---|---|---|---|
| No. | pH | (mg/ml) | (mM) | (mM) | (mM) | (mM) | (mM) | (mM) | PS 80% |
| 1* | 5.5 | 20 | 20 | 0 | 270 | 0 | 0 | 0 | 0.02 |
| 2 | 5.5 | 20 | 10 | 0 | 230 | 0 | 0 | 25 | 0.02 |
| 3 | 5.5 | 20 | 0 | 10 | 0 | 0 | 240 | 25 | 0.02 |
| 4 | 5.5 | 20 | 20 | 0 | 0 | 0 | 270 | 0 | 0.02 |
| 5 | 5.9 | 20 | 20 | 0 | 0 | 0 | 90 | 100 | 0.02 |
| 6 | 5.0 | 20 | 10 | 0 | 180 | 0 | 0 | 50 | 0.02 |
| 7 | 5.9 | 20 | 20 | 0 | 0 | 180 | 0 | 50 | 0.02 |
| 8 | 5.9 | 5 | 0 | 20 | 0 | 80 | 0 | 100 | 0.02 |
| 9 | 5.5 | 20 | 0 | 20 | 110 | 110 | 0 | 25 | 0.02 |
| 10 | 5.5 | 5 | 20 | 0 | 270 | 0 | 0 | 0 | 0.02 |
| 11 | 5.5 | 5 | 0 | 20 | 0 | 0 | 90 | 100 | 0.02 |
| 12 | 5.0 | 5 | 0 | 10 | 180 | 0 | 0 | 50 | 0.02 |

*S2 F02

TABLE 124
pH, Conductivity, UV and Turbidity measurement for Block 4 at T = 0

| Form | Time Point | Temp. | | | UV | Turbidity | |
|---|---|---|---|---|---|---|---|
| No | (Weeks) | C. ° | μs/cm | pH | mg/mL | Slope (400 to 320 nm) | A.I. |
| 1 | 0 | N/A | 1.82 | 5.58 | 20.23 | −1.30E−05 | 0.59 |
| 2 | 0 | N/A | 1.29 | 5.58 | 19.59 | −1.43E−05 | −0.18 |
| 3 | 0 | N/A | 1.44 | 5.66 | 19.83 | −1.85E−05 | 0.10 |
| 4 | 0 | N/A | 1.89 | 5.58 | 20.16 | −2.34E−05 | 0.81 |
| 5 | 0 | N/A | 3.00 | 5.86 | 19.86 | −3.11E−05 | 0.79 |
| 6 | 0 | N/A | 5.86 | 5.15 | 20.15 | −2.75E−05 | 0.38 |
| 7 | 0 | N/A | 6.13 | 5.89 | 20.86 | −1.50E−05 | 0.45 |
| 8 | 0 | N/A | 11.8 | 5.92 | | | |
| 9 | 0 | N/A | 1.79 | 5.55 | 21.13 | −1.42E−05 | 0.13 |
| 10 | 0 | N/A | 1.23 | 5.5 | 4.77 | −1.12E−05 | −1.49 |
| 11 | 0 | N/A | 10.28 | 5.49 | 4.95 | −1.56E−05 | 0.01 |
| 12 | 0 | N/A | 5.56 | 5.23 | 5.11 | −1.18E−05 | 0.53 |

TABLE 125
Visual Inspection for Color and Particles for Block 4 at T = 0

| Form | Time Point | Temp. | Inspection | Inspection |
|---|---|---|---|---|
| No | (Weeks) | C. ° | Color | Particles |
| 1 | 0 | N/A | Clear | Clear |
| 2 | 0 | N/A | Clear | Clear |
| 3 | 0 | N/A | Clear | Clear |
| 4 | 0 | N/A | Clear | Clear |
| 5 | 0 | N/A | Clear | Clear |
| 6 | 0 | N/A | Clear | Clear |
| 7 | 0 | N/A | Clear | Clear |
| 8 | 0 | N/A | Clear | Clear |
| 9 | 0 | N/A | Clear | Clear |
| 10 | 0 | N/A | Clear | Clear |
| 11 | 0 | N/A | Clear | Clear |
| 12 | 0 | N/A | Clear | Clear |

TABLE 126
Reverse Phase data for Block 4 at T = 0

| | | | Reverse Phase | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 0.91 | 99.09 | 0.00 |
| 2 | 0 | N/A | 0.87 | 99.13 | 0.00 |
| 3 | 0 | N/A | 0.87 | 98.99 | 0.14 |
| 4 | 0 | N/A | 0.88 | 99.12 | 0.00 |
| 5 | 0 | N/A | 0.97 | 99.03 | 0.00 |
| 6 | 0 | N/A | 0.94 | 99.06 | 0.00 |
| 7 | 0 | N/A | 0.92 | 99.08 | 0.00 |
| 8 | 0 | N/A | 1.23 | 98.77 | 0.00 |
| 9 | 0 | N/A | 0.82 | 99.18 | 0.00 |
| 10 | 0 | N/A | 0.96 | 99.04 | 0.00 |
| 11 | 0 | N/A | 1.02 | 98.98 | 0.00 |
| 12 | 0 | N/A | 1.19 | 98.62 | 0.19 |

TABLE 127
SEC data for Block 4 T = 0

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 0.21 | 98.80 | 0.99 |
| 2 | 0 | N/A | 0.20 | 98.83 | 0.97 |
| 3 | 0 | N/A | 0.20 | 98.83 | 0.97 |
| 4 | 0 | N/A | 0.19 | 98.85 | 0.96 |
| 5 | 0 | N/A | 0.21 | 98.83 | 0.96 |
| 6 | 0 | N/A | 0.21 | 98.85 | 0.94 |
| 7 | 0 | N/A | 0.23 | 98.82 | 0.96 |
| 8 | 0 | N/A | 0.24 | 98.90 | 0.86 |
| 9 | 0 | N/A | 0.20 | 98.89 | 0.91 |
| 10 | 0 | N/A | 0.13 | 98.96 | 0.91 |
| 11 | 0 | N/A | 0.27 | 98.89 | 0.84 |
| 12 | 0 | N/A | 0.26 | 98.90 | 0.84 |

TABLE 128
HIC data for Block 4 T = 0

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 7.52 | 92.48 | 0.00 |
| 2 | 0 | N/A | 7.58 | 92.42 | 0.00 |
| 3 | 0 | N/A | 7.53 | 92.47 | 0.00 |
| 4 | 0 | N/A | 7.63 | 92.37 | 0.00 |
| 5 | 0 | N/A | 7.89 | 92.11 | 0.00 |
| 6 | 0 | N/A | 7.65 | 92.35 | 0.00 |
| 7 | 0 | N/A | 7.83 | 92.17 | 0.00 |
| 8 | 0 | N/A | 10.27 | 89.73 | 0.00 |
| 9 | 0 | N/A | 7.34 | 92.66 | 0.00 |
| 10 | 0 | N/A | 11.90 | 88.11 | 0.00 |
| 11 | 0 | N/A | 10.71 | 89.29 | 0.00 |
| 12 | 0 | N/A | 9.64 | 90.36 | 0.00 |

TABLE 129
CEX data for Block 4 T = 0

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 0 | N/A | 17.85 | 73.08 | 9.07 |
| 2 | 0 | N/A | 18.03 | 72.66 | 9.32 |
| 3 | 0 | N/A | 17.93 | 72.95 | 9.13 |
| 4 | 0 | N/A | 17.92 | 72.80 | 9.28 |
| 5 | 0 | N/A | 17.85 | 72.89 | 9.26 |
| 6 | 0 | N/A | 17.75 | 72.89 | 9.36 |
| 7 | 0 | N/A | 17.72 | 72.89 | 9.39 |
| 8 | 0 | N/A | 17.38 | 73.48 | 9.15 |
| 9 | 0 | N/A | 17.88 | 72.76 | 9.36 |
| 10 | 0 | N/A | | | |
| 11 | 0 | N/A | 17.53 | 72.62 | 9.85 |
| 12 | 0 | N/A | 17.85 | 72.31 | 9.83 |

TABLE 130
MFI data for Block 4 T = 0

| Form | Time Point | Temp. | Total Particle Count (counts/mL) | | | | |
|---|---|---|---|---|---|---|---|
| No | (Weeks) | C. ° | All | 2 uM | 5 uM | 10 uM | 25 uM |
| 1 | 0 | N/A | 39567 | 10809 | 1819 | 258 | 21 |
| 2 | 0 | N/A | 36594 | 10449 | 1602 | 262 | 46 |
| 3 | 0 | N/A | 42320 | 11128 | 1455 | 133 | 5 |
| 4 | 0 | N/A | 48879 | 13794 | 1411 | 78 | 14 |
| 5 | 0 | N/A | 44294 | 9456 | 513 | 39 | 11 |
| 6 | 0 | N/A | 12319 | 1991 | 162 | 22 | 5 |
| 7 | 0 | N/A | 20714 | 2416 | 357 | 76 | 15 |
| 8 | 0 | N/A | 23301 | 4305 | 664 | 87 | 0 |
| 9 | 0 | N/A | 20374 | 3179 | 151 | 6 | 6 |
| 10 | 0 | N/A | 27050 | 3624 | 192 | 47 | 13 |
| 11 | 0 | N/A | 24563 | 5501 | 320 | 24 | 10 |
| 12 | 0 | N/A | 25089 | 2870 | 169 | 42 | 5 |

TABLE 131
UV and Turbidity measurement for Block 4 for 2 weeks 40 C. °

| Form | Time Point | Temp. | UV | Turbidity | |
|---|---|---|---|---|---|
| No | (Weeks) | C. ° | mg/mL | Slope (400 to 320 nm) | A.I. |
| 1 | 2 | 40 | 19.39 | 1.22E−05 | −1.50 |
| 2 | 2 | 40 | 21.59 | −5.09E−05 | 0.41 |
| 3 | 2 | 40 | 19.10 | −7.64E−06 | −2.26 |
| 4 | 2 | 40 | 19.06 | −4.44E−06 | −2.41 |
| 5 | 2 | 40 | 19.29 | −9.80E−06 | −2.12 |
| 6 | 2 | 40 | 19.24 | −1.14E−05 | −1.81 |
| 7 | 2 | 40 | 20.65 | −2.00E−05 | −1.28 |
| 8 | 2 | 40 | 4.49 | 6.30E−09 | −10.21 |
| 9 | 2 | 40 | 20.27 | −7.77E−06 | −2.20 |
| 10 | 2 | 40 | 4.23 | 4.41E−07 | −9.12 |
| 11 | 2 | 40 | 4.47 | −1.92E−06 | −9.77 |
| 12 | 2 | 40 | 4.43 | 5.27E−06 | −9.80 |

TABLE 132
Visual Inspection for Color and Particles
for Block 4 for 2 weeks 40 C. °

| Form | Time Point | Temp. | Inspection | Inspection |
|---|---|---|---|---|
| No | (Weeks) | C. ° | Color | Particles |
| 1 | 2 | 40 | Clear | Clear |
| 2 | 2 | 40 | Clear | Clear |
| 3 | 2 | 40 | Clear | Clear |
| 4 | 2 | 40 | Clear | Clear |
| 5 | 2 | 40 | Clear | Clear |
| 6 | 2 | 40 | Clear | Clear |
| 7 | 2 | 40 | Clear | Clear |
| 8 | 2 | 40 | Clear | Clear |
| 9 | 2 | 40 | Clear | Clear |
| 10 | 2 | 40 | Clear | Clear |
| 11 | 2 | 40 | Clear | Clear |
| 12 | 2 | 40 | Clear | Clear |

TABLE 133
Reverse Phase data for Block 4 for 2 weeks 40 C. °

| | | | RP | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 1.26 | 98.74 | 0.00 |
| 2 | 2 | 40 | 2.25 | 97.75 | 0.00 |
| 3 | 2 | 40 | 1.14 | 98.86 | 0.00 |
| 4 | 2 | 40 | 1.48 | 98.52 | 0.00 |
| 5 | 2 | 40 | 1.57 | 98.44 | 0.00 |
| 6 | 2 | 40 | 1.30 | 98.70 | 0.00 |
| 7 | 2 | 40 | 1.85 | 98.15 | 0.00 |
| 8 | 2 | 40 | 1.24 | 98.76 | 0.00 |
| 9 | 2 | 40 | 1.19 | 98.82 | 0.00 |
| 10 | 2 | 40 | 1.10 | 98.90 | 0.00 |
| 11 | 2 | 40 | 1.06 | 98.94 | 0.00 |
| 12 | 2 | 40 | 1.05 | 98.95 | 0.00 |

TABLE 134
SEC data for Block 4 for 2 weeks 40 C. °

| | | | SEC | | |
|---|---|---|---|---|---|
| | | | Before Main | Main | After Main |
| | | | Peak | Peak | Peak |
| Form | Time Point | Temp. | Rel. Area | Rel. | Rel. Area |
| No | (Weeks) | C. ° | (%) | Area (%) | (%) |
| 1 | 2 | 40 | 0.19 | 98.28 | 1.54 |
| 2 | 2 | 40 | 0.15 | 97.92 | 1.93 |
| 3 | 2 | 40 | 0.19 | 98.62 | 1.19 |
| 4 | 2 | 40 | 0.18 | 98.36 | 1.46 |
| 5 | 2 | 40 | 0.23 | 98.24 | 1.54 |
| 6 | 2 | 40 | 0.21 | 98.50 | 1.29 |
| 7 | 2 | 40 | 0.20 | 99.74 | 0.06 |
| 8 | 2 | 40 | 0.17 | 98.48 | 1.35 |
| 9 | 2 | 40 | 0.23 | 98.42 | 1.35 |
| 10 | 2 | 40 | 0.10 | 98.54 | 1.37 |
| 11 | 2 | 40 | 0.28 | 98.60 | 1.13 |
| 12 | 2 | 40 | 0.31 | 98.65 | 1.05 |

TABLE 135
HIC data for Block 4 for 2 weeks 40 C. °

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 9.51 | 90.49 | 0.00 |
| 2 | 2 | 40 | 9.81 | 90.17 | 0.03 |
| 3 | 2 | 40 | 9.13 | 90.87 | 0.00 |
| 4 | 2 | 40 | 9.30 | 90.71 | 0.00 |
| 5 | 2 | 40 | 10.04 | 89.96 | 0.00 |
| 6 | 2 | 40 | 9.01 | 90.98 | 0.01 |
| 7 | 2 | 40 | 8.99 | 91.01 | 0.00 |
| 8 | 2 | 40 | 10.27 | 89.73 | 0.00 |
| 9 | 2 | 40 | 8.60 | 91.41 | 0.00 |
| 10 | 2 | 40 | 11.90 | 88.11 | 0.00 |
| 11 | 2 | 40 | 10.71 | 89.29 | 0.00 |
| 12 | 2 | 40 | 9.64 | 90.36 | 0.00 |

TABLE 136
CEX data for Block 4 for 2 weeks 40 C. °

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 2 | 40 | 23.12 | 66.07 | 10.81 |
| 2 | 2 | 40 | 22.96 | 65.06 | 11.98 |
| 3 | 2 | 40 | 22.49 | 68.23 | 9.28 |
| 4 | 2 | 40 | 21.56 | 68.20 | 10.24 |
| 5 | 2 | 40 | 21.91 | 68.43 | 9.66 |
| 6 | 2 | 40 | 20.27 | 68.34 | 11.39 |
| 7 | 2 | 40 | 21.86 | 68.13 | 10.02 |
| 8 | 2 | 40 | 23.05 | 68.50 | 8.45 |
| 9 | 2 | 40 | 22.50 | 67.27 | 10.22 |
| 10 | 2 | 40 | 21.61 | 69.89 | 4.74 |
| 11 | 2 | 40 | 21.10 | 69.74 | 2.07 |
| 12 | 2 | 40 | 20.67 | 70.17 | 3.85 |

TABLE 137
UV and Turbidity measurement for Block 4 for 4 weeks 25 C. °

| Form | Time Point | Temp. | UV | Turbidity | |
|---|---|---|---|---|---|
| No | (Weeks) | C. ° | mg/mL | Slope (400 to 320 nm) | A.I. |
| 1 | 4 | 25 | 20.13 | −1.40E−05 | −0.26 |
| 2 | 4 | 25 | 23.31 | −9.17E−05 | 3.73 |
| 3 | 4 | 25 | 19.95 | −2.84E−05 | 1.14 |
| 4 | 4 | 25 | 19.91 | −1.50E−05 | −0.27 |
| 5 | 4 | 25 | 20.39 | 1.65E−06 | 0.88 |
| 6 | 4 | 25 | 19.99 | −2.40E−05 | −0.13 |
| 7 | 4 | 25 | 20.96 | −2.35E−05 | −0.19 |
| 8 | 4 | 25 | 5.10 | −1.77E−05 | −2.19 |
| 9 | 4 | 25 | 21.12 | −1.68E−05 | −0.34 |
| 10 | 4 | 25 | 5.67 | −3.08E−05 | 1.23 |
| 11 | 4 | 25 | 5.24 | −2.14E−05 | 1.54 |
| 12 | 4 | 25 | 5.43 | −2.01E−05 | 2.41 |

TABLE 138
Visual Inspection for Color and Particles
for Block 4 for 4 weeks 25 C. °

| Form | Time Point | Temp. | Inspection | Inspection |
|---|---|---|---|---|
| No | (Weeks) | C. ° | Color | Particles |
| 1 | 4 | 25 | Clear | Clear |
| 2 | 4 | 25 | Clear | Clear |
| 3 | 4 | 25 | Clear | Clear |
| 4 | 4 | 25 | Clear | Clear |
| 5 | 4 | 25 | Clear | Clear |
| 6 | 4 | 25 | Clear | Clear |
| 7 | 4 | 25 | Clear | Clear |
| 8 | 4 | 25 | Clear | Clear |
| 9 | 4 | 25 | Clear | Clear |
| 10 | 4 | 25 | Clear | Clear |
| 11 | 4 | 25 | Clear | Clear |
| 12 | 4 | 25 | Clear | Clear |

TABLE 139
SEC data for Block 4 for 4 weeks 25 C. °

| | | | SEC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 0.19 | 98.27 | 1.54 |
| 2 | 4 | 25 | 0.15 | 97.92 | 1.93 |
| 3 | 4 | 25 | 0.19 | 98.62 | 1.19 |
| 4 | 4 | 25 | 0.18 | 98.36 | 1.46 |
| 5 | 4 | 25 | 0.22 | 98.24 | 1.54 |
| 6 | 4 | 25 | 0.21 | 98.50 | 1.29 |
| 7 | 4 | 25 | 0.20 | 99.74 | 0.06 |
| 8 | 4 | 25 | 0.17 | 98.48 | 1.35 |
| 9 | 4 | 25 | 0.23 | 98.42 | 1.35 |
| 10 | 4 | 25 | 0.10 | 98.54 | 1.37 |
| 11 | 4 | 25 | 0.28 | 98.60 | 1.13 |
| 12 | 4 | 25 | 0.31 | 98.65 | 1.04 |

TABLE 140
CEX data for Block 4 for 4 weeks 25 C. °

| | | | CEX | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 17.29 | 73.50 | 9.21 |
| 2 | 4 | 25 | 17.46 | 73.41 | 9.14 |
| 3 | 4 | 25 | 17.08 | 73.62 | 9.30 |
| 4 | 4 | 25 | 16.99 | 73.60 | 9.41 |
| 5 | 4 | 25 | 17.56 | 73.15 | 9.29 |
| 6 | 4 | 25 | 17.08 | 73.16 | 9.77 |
| 7 | 4 | 25 | 17.33 | 73.20 | 9.47 |
| 8 | 4 | 25 | 16.83 | 74.00 | 9.18 |
| 9 | 4 | 25 | 17.24 | 73.33 | 9.43 |
| 10 | 4 | 25 | 15.94 | 74.55 | 9.51 |
| 11 | 4 | 25 | 12.12 | 79.27 | 8.61 |
| 12 | 4 | 25 | 17.19 | 73.17 | 9.64 |

TABLE 141
Reverse Phase data for Block 4 for 4 weeks 25 C. °

| | | | RP | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 1.01 | 98.99 | 0.00 |
| 2 | 4 | 25 | 1.58 | 98.42 | 0.00 |
| 3 | 4 | 25 | 1.01 | 98.99 | 0.00 |
| 4 | 4 | 25 | 1.07 | 98.93 | 0.00 |
| 5 | 4 | 25 | 1.06 | 98.94 | 0.00 |
| 6 | 4 | 25 | 1.03 | 98.97 | 0.00 |
| 7 | 4 | 25 | 1.22 | 98.78 | 0.00 |
| 8 | 4 | 25 | 0.97 | 99.03 | 0.00 |
| 9 | 4 | 25 | 0.98 | 99.02 | 0.00 |
| 10 | 4 | 25 | 1.01 | 98.99 | 0.00 |
| 11 | 4 | 25 | 0.91 | 99.09 | 0.00 |
| 12 | 4 | 25 | 0.93 | 99.07 | 0.00 |

TABLE 142
HIC data for Block 4 for 4 weeks 25 C. °

| | | | HIC | | |
|---|---|---|---|---|---|
| | | | Before Main | Main | After Main |
| | | | Peak | Peak | Peak |
| Form | Time Point | Temp. | Rel. Area | Rel. Area | Rel. Area |
| No | (Weeks) | C. ° | (%) | (%) | (%) |
| 1 | 4 | 25 | 8.72 | 91.28 | 0.00 |
| 2 | 4 | 25 | 9.10 | 90.90 | 0.00 |
| 3 | 4 | 25 | 8.68 | 91.32 | 0.00 |
| 4 | 4 | 25 | 8.73 | 91.27 | 0.00 |
| 5 | 4 | 25 | 9.28 | 90.72 | 0.00 |
| 6 | 4 | 25 | 8.57 | 91.43 | 0.00 |
| 7 | 4 | 25 | 8.46 | 91.54 | 0.00 |
| 8 | 4 | 25 | 8.90 | 91.10 | 0.00 |
| 9 | 4 | 25 | 8.33 | 91.67 | 0.00 |
| 10 | 4 | 25 | 9.12 | 90.88 | 0.00 |
| 11 | 4 | 25 | 9.65 | 90.35 | 0.00 |
| 12 | 4 | 25 | 8.48 | 91.52 | 0.00 |

TABLE 143
Flow imaging data for Block 4 for 4 weeks 25 C. °

| Form | Time Point | Temp. | Total Particle Count (counts/mL) | | | | |
|---|---|---|---|---|---|---|---|
| No | (Weeks) | C. ° | All | 2 uM | 5 uM | 10 uM | 25 uM |
| 1 | 4 | 25 | 23462 | 5669 | 1074 | 283 | 93 |
| 2 | 4 | 25 | 20535 | 4080 | 633 | 89 | 6 |
| 3 | 4 | 25 | 10370 | 1644 | 237 | 100 | 37 |
| 4 | 4 | 25 | 19486 | 2431 | 294 | 62 | 4 |
| 5 | 4 | 25 | 26091 | 3972 | 297 | 88 | 13 |
| 6 | 4 | 25 | 12629 | 1783 | 290 | 45 | 27 |
| 7 | 4 | 25 | 36586 | 5880 | 731 | 79 | 0 |
| 8 | 4 | 25 | 9760 | 969 | 188 | 75 | 33 |
| 9 | 4 | 25 | 43357 | 6774 | 605 | 59 | 14 |
| 10 | 4 | 25 | 1286166 | 429068 | 171265 | 46386 | 294 |
| 11 | 4 | 25 | 7841 | 656 | 78 | 7 | 0 |
| 12 | 4 | 25 | 13663 | 1634 | 268 | 44 | 0 |

Upon storage, little, if any, change is seen RP HPLC or SEC for any of these formulations. There are some changes by CEX for the 40° C. samples (Table 136). The losses seem to be most pronounced for sucrose-containing formulations. By HIC, the 5 mg/ml samples may be less stable at 40 C (Table 135).

For material held at 25° C., there are no appreciable changes by CEX, SEC, HIC, or RP HPLC over the course of four weeks. Overall, these data indicate that the compositions selected here have long-term stability as evidenced by the above-described short-term stability studies.

Some of these formulations were subjected to F/T cycling. No changes were observed by any of the chromatographic methods.

TABLE 144
UV and Turbidity measurement for Block 4
for Three Freeze Thaw −20° C.

| Form | Freeze | Temp. | UV | Turbidity | |
|---|---|---|---|---|---|
| No | Thaws | C. ° | mg/mL | Slope (400 to 320 nm) | A.I. |
| 1 | 4 | 25 | 19.90 | −1.71E−05 | −0.03 |
| 2 | 4 | 25 | 19.67 | −1.28E−05 | −0.48 |
| 3 | 4 | 25 | 19.90 | −3.33E−05 | 0.83 |
| 4 | 4 | 25 | 19.84 | −1.78E−05 | 0.45 |
| 5 | 4 | 25 | 19.74 | −1.42E−05 | −0.06 |
| 6 | 4 | 25 | 19.85 | −6.87E−06 | −0.53 |
| 7 | 4 | 25 | 20.83 | −9.22E−06 | −0.12 |
| 9 | 4 | 25 | 21.16 | −1.18E−05 | −0.04 |

TABLE 145
SEC data for Block 4 for Three Freeze Thaw −20 C. °

| | | | SEC | | |
|---|---|---|---|---|---|
| | | | Before Main | Main | After Main |
| | | | Peak | Peak | Peak |
| Form | Freeze | Temp. | Rel. Area | Rel. Area | Rel. Area |
| No | Thaws | C. ° | (%) | (%) | (%) |
| 1 | 3 | −20 | 0.16 | 98.93 | 0.91 |
| 2 | 3 | −20 | 0.16 | 98.93 | 0.91 |
| 3 | 3 | −20 | 0.16 | 98.84 | 1.01 |
| 4 | 3 | −20 | 0.18 | 98.86 | 0.96 |
| 5 | 3 | −20 | 0.25 | 98.67 | 1.09 |
| 6 | 3 | −20 | 0.20 | 98.87 | 0.93 |
| 7 | 3 | −20 | 0.22 | 98.78 | 1.00 |
| 9 | 3 | −20 | 0.20 | 99.78 | 0.03 |

TABLE 146
CEX data for Block 4 for Three Freeze Thaw −20 C. °

| | | | CEX | | |
|---|---|---|---|---|---|
| | | | Before Main | Main | After Main |
| | | | Peak | Peak | Peak |
| Form | Freeze | Temp. | Rel. Area | Rel. Area | Rel. Area |
| No | Thaws | C. ° | (%) | (%) | (%) |
| 1 | 3 | −20 | 17.79 | 72.63 | 9.58 |
| 2 | 3 | −20 | 17.72 | 72.90 | 9.39 |
| 3 | 3 | −20 | 17.64 | 72.69 | 9.67 |
| 4 | 3 | −20 | 17.57 | 72.95 | 9.48 |
| 5 | 3 | −20 | 17.55 | 72.91 | 9.54 |
| 6 | 3 | −20 | 17.48 | 72.74 | 9.78 |
| 7 | 3 | −20 | 17.06 | 73.43 | 9.51 |
| 9 | 3 | −20 | 16.87 | 73.51 | 9.62 |

TABLE 147
Reverse Phase data for Block 4 for Three Freeze Thaw −20 C. °

| | | | RP | | |
|---|---|---|---|---|---|
| | | | Before Main | Main | After Main |
| | | | Peak | Peak | Peak |
| Form | Freeze | Temp. | Rel. Area | Rel. Area | Rel. Area |
| No | Thaws | C. ° | (%) | (%) | (%) |
| 1 | 3 | −20 | 0.84 | 99.16 | 0.00 |
| 2 | 3 | −20 | 1.02 | 98.98 | 0.00 |
| 3 | 3 | −20 | 0.91 | 99.09 | 0.00 |
| 4 | 3 | −20 | 0.76 | 99.14 | 0.10 |
| 5 | 3 | −20 | | | |
| 6 | 3 | −20 | 0.88 | 99.12 | 0.00 |
| 7 | 3 | −20 | 0.89 | 99.12 | 0.00 |
| 9 | 3 | −20 | 0.93 | 99.07 | 0.00 |

TABLE 148
HIC data for Block 4 for Three Freeze Thaw −20 C. °

| | | | HIC | | |
|---|---|---|---|---|---|
| | | | Before Main | Main | After Main |
| | | | Peak | Peak | Peak |
| Form | Freeze | Temp. | Rel. Area | Rel. Area | Rel. Area |
| No | Thaws | C. ° | (%) | (%) | (%) |
| 1 | 3 | −20 | 8.07 | 91.93 | 0.00 |
| 2 | 3 | −20 | 8.15 | 91.85 | 0.00 |
| 3 | 3 | −20 | 8.03 | 91.97 | 0.00 |
| 4 | 3 | −20 | 7.85 | 92.15 | 0.00 |
| 5 | 3 | −20 | 8.44 | 91.56 | 0.00 |
| 6 | 3 | −20 | 8.02 | 91.98 | 0.00 |
| 7 | 3 | −20 | 7.90 | 92.10 | 0.00 |
| 9 | 3 | −20 | 7.91 | 92.09 | 0.00 |

TABLE 149
UV and Turbidity measurement for Block 4
for Five Freeze Thaw −20 C. °

| Form | Freeze | Temp. | UV | Turbidity | |
|---|---|---|---|---|---|
| No | Thaws | C. ° | mg/mL | Slope (400 to 320 nm) | A.I. |
| 1 | 5 | −20 | 20.84 | −8.79E−06 | 0.78 |
| 2 | 5 | −20 | 27.99 | −8.02E−05 | 3.75 |
| 3 | 5 | −20 | 20.96 | −2.27E−05 | 1.21 |
| 4 | 5 | −20 | 20.70 | −6.11E−06 | 0.73 |
| 5 | 5 | −20 | 20.55 | −1.28E−05 | 1.01 |
| 6 | 5 | −20 | 20.65 | −6.47E−06 | 0.56 |
| 7 | 5 | −20 | 21.97 | −4.98E−06 | 0.91 |
| 9 | 5 | −20 | 22.29 | −1.13E−05 | 3.23 |

TABLE 150
Visual Inspection for Color and Particles for
Block 4 for Five Freeze Thaw −20 C. °

| Form | Freeze | Temp. | Inspection | Inspection |
|---|---|---|---|---|
| No | Thaws | C. ° | Color | Particles |
| 1 | 5 | −20 | Clear | Clear |
| 2 | 5 | −20 | White | Cloudy |
| 3 | 5 | −20 | Clear | Clear |
| 4 | 5 | −20 | Clear | Clear |
| 5 | 5 | −20 | Clear | Clear |
| 6 | 5 | −20 | Clear | Clear |
| 9 | 5 | −20 | Clear | Clear |

TABLE 151
SEC data for Block 4 for Five Freeze Thaw −20 C. °

| | | | SEC | | |
|---|---|---|---|---|---|
| | | | Before Main | Main | After Main |
| | | | Peak | Peak | Peak |
| Form | Freeze | Temp. | Rel. Area | Rel. Area | Rel. Area |
| No | Thaws | C. ° | (%) | (%) | (%) |
| 1 | 5 | −20 | 0.10 | 98.80 | 1.10 |
| 2 | 5 | −20 | 0.11 | 98.55 | 1.33 |
| 3 | 5 | −20 | 0.12 | 98.82 | 1.06 |
| 4 | 5 | −20 | 0.11 | 98.76 | 1.13 |
| 5 | 5 | −20 | 0.15 | 98.83 | 1.02 |
| 6 | 5 | −20 | 0.15 | 98.79 | 1.06 |
| 7 | 5 | −20 | 0.15 | 98.83 | 1.03 |
| 9 | 5 | −20 | 0.14 | 98.85 | 1.01 |

TABLE 152
CEX data for Block 4 for Five Freeze Thaw −20 C. °

| | | | CEX | | |
|---|---|---|---|---|---|
| | | | Before Main | Main | After Main |
| | | | Peak | Peak | Peak |
| Form | Freeze | Temp. | Rel. Area | Rel. Area | Rel. Area |
| No | Thaws | C. ° | (%) | (%) | (%) |
| 1 | 5 | −20 | 17.34 | 73.42 | 9.24 |
| 2 | 5 | −20 | 18.67 | 72.51 | 8.82 |
| 3 | 5 | −20 | 17.38 | 73.12 | 9.50 |
| 4 | 5 | −20 | 16.94 | 73.79 | 9.27 |
| 5 | 5 | −20 | 17.10 | 73.60 | 9.29 |
| 6 | 5 | −20 | 17.12 | 73.38 | 9.51 |
| 7 | 5 | −20 | 17.08 | 73.49 | 9.43 |
| 9 | 3 | −20 | 16.87 | 73.74 | 9.40 |

TABLE 153
UV and Turbidity measurement for Block
4 for Control for −40 C. °

| Form | Freeze | Temp. | UV | Turbidity | |
|---|---|---|---|---|---|
| No | Thaws | C. ° | mg/mL | Slope (400 to 320 nm) | A.I. |
| 1 | 0 | Control | 20.03 | −2.10E−05 | −0.01 |
| 2 | 0 | Control | 25.42 | −7.37E−05 | 2.78 |
| 3 | 0 | Control | 20.01 | −2.02E−05 | 0.07 |
| 4 | 0 | Control | 20.76 | −2.85E−05 | 1.74 |
| 5 | 0 | Control | 20.18 | −1.57E−05 | 0.13 |
| 6 | 0 | Control | 20.24 | −1.09E−05 | 0.07 |
| 7 | 0 | Control | 21.36 | −8.39E−06 | 0.81 |
| 8 | 0 | Control | 5.33 | −6.81E−06 | 1.76 |
| 9 | 0 | Control | 21.26 | −1.86E−05 | −0.02 |
| 10 | 0 | Control | 5.26 | −2.44E−05 | 2.71 |
| 11 | 0 | Control | 5.05 | −2.73E−07 | −0.39 |
| 12 | 0 | Control | 5.31 | −3.83E−06 | 1.87 |

TABLE 154
Visual Inspection for Color and Particles for
Block 4 for Control for −40 C. °

| Form | Freeze | Temp. | Inspection | Inspection |
|---|---|---|---|---|
| No | Thaws | C. ° | Color | Particles |
| 1 | 0 | Control | Clear | Clear |
| 2 | 0 | Control | White | Cloudy |
| 3 | 0 | Control | Clear | Clear |
| 4 | 0 | Control | Clear | Clear |
| 5 | 0 | Control | Clear | Cloudy |
| 6 | 0 | Control | Clear | Clear |
| 7 | 0 | Control | Clear | Clear |
| 8 | 0 | Control | Clear | Clear |
| 9 | 0 | Control | Clear | Clear |
| 10 | 0 | Control | Clear | Clear |
| 11 | 0 | Control | Clear | Clear |
| 12 | 0 | Control | Clear | Clear |

TABLE 155
SEC data for Block 4 for Control for −40 C. °

| | | | SEC | | |
|---|---|---|---|---|---|
| | | | Before Main | Main | After Main |
| | | | Peak | Peak | Peak |
| Form | Freeze | Temp. | Rel. Area | Rel. Area | Rel. Area |
| No | Thaws | C. ° | (%) | (%) | (%) |
| 1 | 0 | Control | 0.12 | 98.88 | 1.00 |
| 2 | 0 | Control | 0.12 | 98.26 | 1.62 |
| 3 | 0 | Control | 0.11 | 98.92 | 0.96 |
| 4 | 0 | Control | 0.12 | 98.92 | 0.97 |
| 5 | 0 | Control | 0.15 | 98.87 | 0.98 |
| 6 | 0 | Control | 0.13 | 98.90 | 0.97 |
| 7 | 0 | Control | 0.17 | 98.93 | 0.89 |
| 8 | 0 | Control | 0.13 | 98.91 | 0.97 |
| 9 | 0 | Control | 0.10 | 98.93 | 0.97 |
| 10 | 0 | Control | 0.10 | 99.00 | 0.90 |
| 11 | 0 | Control | 0.15 | 98.98 | 0.88 |
| 12 | 0 | Control | 0.13 | 98.97 | 0.90 |

TABLE 156
UV and Turbidity measurement for Block 4
Three Freeze Thaw for −40 C. °

| Form | Freeze | Temp. | UV | Turbidity | |
|---|---|---|---|---|---|
| No | Thaws | C. ° | mg/mL | Slope (400 to 320 nm) | A.I. |
| 1 | 3 | −40 | 20.14 | −1.43E−05 | 0.06 |
| 2 | 3 | −40 | 25.19 | −3.62E−05 | 0.51 |
| 3 | 3 | −40 | 20.08 | −3.17E−06 | −0.06 |
| 4 | 3 | −40 | 18.87 | 2.51E−07 | 0.12 |
| 5 | 3 | −40 | 20.27 | −1.71E−05 | 1.06 |
| 6 | 3 | −40 | 20.06 | −8.31E−06 | 0.01 |
| 7 | 3 | −40 | 21.31 | −2.25E−05 | 0.46 |
| 8 | 3 | −40 | 5.38 | −7.17E−06 | 1.55 |
| 9 | 3 | −40 | 21.42 | −2.61E−05 | 0.40 |
| 10 | 3 | −40 | 4.95 | −9.82E−06 | 0.05 |
| 11 | 3 | −40 | 4.88 | 2.81E−06 | 0.51 |
| 12 | 3 | −40 | 5.33 | −1.49E−05 | 0.69 |

TABLE 157
Visual Inspection for Color and Particles for Block
4 Three Freeze Thaw for −40 C. °

| Form | Freeze | Temp. | Inspection | Inspection |
|---|---|---|---|---|
| No | Thaws | C. ° | Color | Particles |
| 1 | 3 | −40 | Clear | Clear |
| 2 | 3 | −40 | Clear | Clear |
| 3 | 3 | −40 | Clear | Clear |
| 4 | 3 | −40 | Clear | Clear |
| 5 | 3 | −40 | Clear | Cloudy |
| 6 | 3 | −40 | Clear | Clear |
| 7 | 3 | −40 | Clear | Clear |
| 8 | 3 | −40 | Clear | Clear |
| 9 | 3 | −40 | Clear | Clear |
| 10 | 3 | −40 | Clear | Clear |
| 11 | 3 | −40 | Clear | Clear |
| 12 | 3 | −40 | Clear | Clear |

TABLE 158
SEC data for Block 4 Block 4 Three Freeze Thaw for −40 C. °

| | | | SEC | | |
|---|---|---|---|---|---|
| | | | Before Main | Main | After Main |
| | | | Peak | Peak | Peak |
| Form | Freeze | Temp. | Rel. Area | Rel. Area | Rel. Area |
| No | Thaws | C. ° | (%) | (%) | (%) |
| 1 | 3 | −40 | 0.13 | 98.62 | 1.25 |
| 2 | 3 | −40 | 0.13 | 98.75 | 1.12 |
| 3 | 3 | −40 | 0.12 | 98.96 | 0.92 |
| 4 | 3 | −40 | 0.13 | 98.95 | 0.92 |
| 5 | 3 | −40 | 0.14 | 98.99 | 0.87 |
| 6 | 3 | −40 | 0.15 | 98.91 | 0.94 |
| 7 | 3 | −40 | 0.14 | 98.90 | 0.96 |
| 8 | 3 | −40 | 0.12 | 98.96 | 0.91 |
| 9 | 3 | −40 | 0.11 | 99.00 | 0.90 |
| 10 | 3 | −40 | 0.12 | 99.01 | 0.87 |
| 11 | 3 | −40 | 0.21 | 98.90 | 0.89 |
| 12 | 3 | −40 | 0.19 | 98.90 | 0.90 |

TABLE 159
CEX data for Block 4 Block 4 Three Freeze Thaw for −40 C. °

| | | | CEX | | |
|---|---|---|---|---|---|
| | | | Before Main | Main | After Main |
| | | | Peak | Peak | Peak |
| Form | Freeze | Temp. | Rel. Area | Rel. Area | Rel. Area |
| No | Thaws | C. ° | (%) | (%) | (%) |
| 1 | 3 | −40 | 20.66 | 69.32 | 10.02 |
| 2 | 3 | −40 | 23.24 | 66.95 | 9.81 |
| 3 | 3 | −40 | 20.71 | 69.65 | 9.65 |
| 4 | 3 | −40 | 20.86 | 69.19 | 9.95 |
| 5 | 3 | −40 | 20.98 | 68.85 | 10.17 |
| 6 | 3 | −40 | 20.66 | 68.93 | 10.41 |
| 7 | 3 | −40 | 21.00 | 68.77 | 10.22 |
| 8 | 3 | −40 | 20.12 | 70.33 | 9.55 |
| 9 | 3 | −40 | 20.86 | 68.60 | 10.54 |
| 10 | 3 | −40 | 20.21 | 71.52 | 8.28 |
| 11 | 3 | −40 | 20.35 | 70.09 | 9.56 |
| 12 | 3 | −40 | 20.46 | 69.85 | 9.69 |

### Example 7—Sub-Visible Particles in Natalizumab Formulations

This example describes methods which may be used to quantify sub-visible particles (SVPs), which are one indication of quality of the formulation (fewer particles=higher quality).

Method 1. Light Obscuration Particle Count Test

This method is based on the principle of light blockage which allows an automatic determination of the size of particles and the number of particles according to size. A suitable apparatus may be calibrated using dispersions of spherical particles of known sizes between 10 μm and 25 μm, USP Particle Count Reference Standard. These standard particles are dispersed in particle-free water.

Method 2. Microscopic Particle Count Test

This method uses a suitable binocular microscope, filter assembly for retaining particulate matter and membrane filter for examination. The microscope is typically equipped with an ocular micrometer calibrated with an objective micrometer, a mechanical stage capable of holding and traversing the entire filtration area of the membrane filter, two suitable illuminators to provide episcopic illumination in addition to oblique illumination, and is typically adjusted to 100±10 magnifications.

Tested via either method, formulations of the present invention preferably comply with USP standard <788> (Particulate Matter In Injections), as follows.

Solutions for parenteral infusion or solutions for injection supplied in containers with a nominal content of more than 100 mL comply with the test if the average number of particles present in the units tested does not exceed 12 per mL equal to or greater than 10 μm and does not exceed 2 per mL equal to or greater than 25 μm.

Solutions for parenteral infusion or solutions for injection supplied in containers with a nominal content of less than or equal to 100 mL comply with the test if the average number of particles present in the units tested does not exceed 3000 per container equal to or greater than 10 μm and does not exceed 300 per container equal to or greater than 25 μm., e.g., fewer than 6000 particles per container 10 μm particles) and fewer than 600 particles per container 25 μm particles).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An aqueous formulation comprising 20 mg/mL natalizumab and a buffer having a pH of between 4 and 7, wherein said buffer is not a phosphate buffer.

2. The formulation of claim 1, wherein said buffer is selected from the group consisting of acetate, succinate, citrate and histidine.

3. The formulation of claim 2, wherein said formulation further comprises a surfactant.

4. The formulation of claim 5, wherein said surfactant is selected from the group consisting of polysorbate 20 (PS-20), polysorbate 80 (PS-80) and n-Dodecyl-3-D-maltopyranoside (DDM).

5. The formulation of claim 2, comprising 20 mM acetate at pH 5.0, and wherein the formulation further comprises 0.02% PS-20.

6. The formulation of claim 2, comprising 20 mM succinate at pH 5.0, and wherein the formulation further comprises 0.02% P5-20.

7. The formulation of claim 2, comprising 20 mM citrate at pH 5.0, and wherein the formulation further comprises 0.02% PS-20.

8. The formulation of claim 2, comprising 20 mM histidine at pH 5.0, and wherein the formulation further comprises 0.02% PS-20.

9. The formulation of claim 2, comprising 20 mM acetate at pH 5.0, and wherein the formulation further comprises 0.1% DDM.

10. The formulation of claim 2, comprising 20 mM succinate at pH 5.0, and wherein the formulation further comprises 0.1% DDM.

11. The formulation of claim 2, comprising 20 mM citrate at pH 5.0, and wherein the formulation further comprises 0.1% DDM.

12. The formulation of claim 2, comprising 20 mM histidine at pH 5.0, and wherein the formulation further comprises 0.1% DDM.

13. The formulation of claim 2, comprising 20 mM acetate at pH 5.5, and wherein the formulation further comprises 0.02% PS-20.

14. The formulation of claim 2, comprising 20 mM succinate at pH 5.5, and wherein the formulation further comprises 0.02% PS-20.

15. The formulation of claim 2, comprising 20 mM citrate at pH 5.5, and wherein the formulation further comprises 0.02% PS-20.

16. The formulation of claim 2, comprising 20 mM histidine at pH 5.5, and wherein the formulation further comprises 0.02% P5-20.

17. The formulation of claim 2, comprising 20 mM acetate at pH 5.5, and wherein the formulation further comprises 0.1% DDM.

18. The formulation of claim 2, comprising 20 mM succinate at pH 5.5, and wherein the formulation further comprises 0.1% DDM.

19. The formulation of claim 2, comprising 20 mM citrate at pH 5.5, and wherein the formulation further comprises 0.1% DDM.

20. The formulation of claim 2, comprising 20 mM histidine at pH 5.5, and wherein the formulation further comprises 0.1% DDM.
